(19) United States
(12) Patent Application Publication
(10) Pub. No.: US 2009/0111745 A1
(43) Pub. Date: Apr. 30, 2009

(54) Plad Domain Peptides With Increased Serum Half Life Due To Conjugation To Domain Antibodies

(75) Inventor: Ian M. Tomlinson, Great Shelford (GB)

Correspondence Address: MCDERMOTT WILL & EMERY LLP, 28 STATE STREET, BOSTON, MA, 02109-1775, US

(73) Assignee: DOMANTIS LIMITED, Cambridge (GB)

(21) Appl. No.: 11/791,399

(22) PCT Filed: Dec. 1, 2005
(86) PCT No.: PCT/GB2005/004603

§ 371 Date: Oct. 31, 2007

Related U.S. Application Data
(60) Provisional application No. 60/632,361, filed on Dec. 2, 2004.

(51) Int. Cl. A61K 38/02; C07K 16/18; C12N 15/11; C12N 15/00; C12N 5/06; C12P 21/04

(52) U.S. Cl. 514/12; 530/387.3; 536/23.53; 435/320.1; 435/369; 435/358; 435/365; 435/69.7

(57) ABSTRACT

Drug fusions and conjugates that contain a therapeutic or diagnostic agent that is fused or conjugated to an antigen-binding fragment of an antibody that binds serum albumin. The conjugates and fusion have a longer in vivo half life in comparison with the unconjugated or unfused therapeutic or diagnostic agent.

### RELATED APPLICATIONS

This application is a continuation-in-part of International Application No. PCT/GB2005/004319, which designated the United States and was filed on Nov. 10, 2005; and is a continuation-in-part of International Application No. PCT/GB2005/002163, which designated the United States and was filed on May 31, 2005, which claims the benefit of U.S. Provisional Patent Application No. 60/632,361, filed on Dec. 2, 2004. The entire teachings of the above applications are incorporated herein by reference.

### BACKGROUND OF THE INVENTION

Many drugs that possess activities that could be useful for therapeutic and/or diagnostic purposes have limited value because they are rapidly eliminated from the body when administered. For example, many polypeptides that have therapeutically useful activities are rapidly cleared from the circulation via the kidney. Accordingly, a large dose must be administered in order to achieve a desired therapeutic effect. A need exists for improved therapeutic and diagnostic agents that have improved pharmacokinetic properties. Polypeptides that bind serum albumin axe known in the art. (See, e.g., EP 0486525 B1 (Cemu Bioteknik AB); U.S. Pat. No. 6,267,964 B1 (Nygren et al.) WO 04/001064 A2 (Dyax, Corp.); WO 02/076489 A1 (Dyax, Corp.); WO 01/45746 (Genentech, Inc.).)

### SUMMARY OF THE INVENTION

The invention relates to drug compositions, fusions and conjugates that contain a PLAD domain or functional variant of a PLAD domain. In one aspect, the invention is a drug fusion comprising moieties X′ and Y″, wherein X′ is a PLAD domain or functional variant of a PLAD domain; and Y′ is polypeptide binding moiety having a bidding site that has binding specificity for a polypeptide that enhances scrum, half-life in vivo.

In some embodiments, the polypeptide binding moiety has binding specificity for serum albumin. For example, the polypeptide binding moiety can be an antigen-binding fragment of an antibody that has binding specificity for serum albumin.

The PLAD domain or functional variant of a PLAD domain preferably comprises a region of at least about 10 contiguous amino acids that are the same as the amino acids in the amino acid sequence of a PLAD domain selected from the PLAD domains of TNFR1, TNFR2, FAS, LT βR, CD40, CD30, CD27, HVEM, OX40, and DR4. For example, the amino acid sequence of the PLAD domain or functional variant of a PLAD domain can have at least about 90% amino acid sequence identity with the amino acid sequence of a PLAD domain selected from the PLAD domains of TNFR1, TNFR2, FAS, LT βR, CD40, CD30, CD27, HVEM, OX40, and DR4, In another example, the amino acid sequence of said PLAD domain or functional variant of a PLAD domain has at least about 90% amino acid sequence identity with an amino acid sequence selected from the group consisting of SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97.

In some embodiments, the drug fusion comprises moieties X′ and Y′ wherein X′ is a PLAD domain or functional variant of a PLAD domain; and Y′ is an immunoglobulin heavy chain variable domain that has binding specificity for serum albumin, or an immunoglobulin light chain variable domain that has binding specificity for serum albumin. In such embodiments, X′ can be located amino terminally to Y′, or Y′ can be located amino terminally to X′. Preferably, the heavy chain variable domain and light chain variable domain have binding specificity for human serum albumin.

In certain embodiments, Y′ comprises an amino acid sequence selected from the group consisting of SEQ ID NO:10, SEQ ID NO:11, SEQ ID NO:12, SEQ ID NO:13, SEQ ID NO:14, SEQ ID NO:15, SEQ ID NO:24, SEQ ID NO:25 and SEQ ID NO:26.

In other embodiments, Y′ comprises an amino acid sequence selected from the group consisting of SEQ ID NO:16, SEQ ID NO:17, SEQ ID NO:18, SEQ ID NO:19, SEQ ID NO:20, SEQ ID NO:21, SEQ ID NO:22 and SEQ ID NO:23.

The PLAD domain or functional variant of a PLAD domain preferably comprises a region of at least about 10 contiguous amino acids that are the same as the amino acids in the amino acid sequence of a PLAD domain selected from the PLAD domains of TNFR1, TNFR2, FAS, LT βR, CD40, CD30, CD27, HVEM, OX40, and DR4. For example, the amino acid sequence of the PLAD domain or functional variant of a PLAD domain can have at least about 90% amino acid sequence identity with the amino acid sequence of a PLAD domain selected from the PLAD domains of TNFR1, TNFR2, FAS, LT βR, CD40, CD30, CD27, HVEM, OX40, and DR4. In another example, the amino acid sequence of said PLAD domain or functional variant of a PLAD domain has at least about 90% amino acid sequence identity with an amino acid sequence selected from the group consisting of SEQ ID NO:87, SEQ ID NO:88, SEQ ED NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97.

In other aspects, the invention is a drug conjugate comprising an immunoglobulin heavy chain variable domain that has binding specificity for serum albumin, or an immunoglobulin light chain variable domain that has binding specificity for serum albumin, and a PLAD domain or functional variant of a PLAD domain mat is covalently bonded to said immunoglobulin heavy chain variable domain or immunoglobulin light chain variable domain. In some embodiment, the PLAD domain or functional variant of a PLAD domain is covalently bonded to said immunoglobulin heavy chain variable domain or immunoglobulin light chain variable domain through a linker moiety.

In certain embodiments, the immunoglobulin heavy chain variable domain that has binding specificity for serum albumin, or the immunoglobulin light chain variable domain that has binding specificity for serum albumin comprises an amino acid sequence selected from the group consisting of SEQ ID NO:10, SEQ ID NO:11, SEQ ID NO:12, SEQ ID NO:13, SEQ ID NO:14, SEQ ID NO:15, SEQ ID NO:24, SEQ ID NO:25, SEQ ID NO:26, SEQ ID NO:15, SEQ ID NO:17, SEQ ID NO:18, SEQ ID NO:19, SEQ ID NO:20, SEQ ID NO:21, SEQ ID NO:22 and SEQ ID NO:23.

The PLAD domain or functional variant of a PLAD domain preferably comprises a region of at least about 10 contiguous amino acids that are the same as the amino acids in the amino acid sequence of a PLAD domain selected from the PLAD domains of TNFR1, TNFR2, FAS, LT βR, CD40, CD30, CD27, HVEM, OX40, and DR4. For example, the amino acid sequence of the PLAD domain or functional variant of a PLAD domain can have at least about 90% amino acid sequence identity with the amino acid sequence of a PLAD domain selected from the PLAD domains of TNFR1, TNFR2, FAS, LT βR, CD40, CD30, CD27, HVEM, OX40, and DR4. In another example, the amino acid sequence of said PLAD domain or functional variant of a PLAD domain has at least about 90% amino acid sequence identity with an amino acid sequence selected from the group consisting of SEQ ID NO:87, SEQ ID NO:88, SEQ ID NO:89, SEQ ID NO:90, SEQ ID NO:91, SEQ ID NO:92, SEQ ID NO:93, SEQ ID NO: 4, SEQ ID NO:95, SEQ ID NO:96, and SEQ ID NO:97.

The invention also relates to an isolated or recombinant nucleic acid and nucleic acid constructs encoding the drug fusions of the invention. The invention also relates to a host cell comprising the recombinant nucleic acid of the invention, and to a method for producing a drag fusion comprising maintaining the host cell under conditions suitable for expression of said recombinant nucleic acid, whereby a drag fusion is produced.

The invention also relates to a pharmaceutical composition comprising a drug fusion of or drug conjugate of the invention and a physiologically acceptable carrier.

The invention also relates to a method for treating an individual having an inflammatory disease, comprising administering to said individual a therapeutically effective amount of a drug conjugate or drug fusion of the invention. In particular embodiments, the inflammatory disease is arthritis. The invention also relates to drug conjugate or drug fusion use in therapy, diagnosis or prophylaxis, and to the use of a drug conjugate or dug fusion of the invention for the manufacture of a medicament for treating an inflammatory disease, such as the diseases disclosed herein (e.g., arthritis).

The invention also relates to a drug composition comprising a PLAD domain or functional variant of a PLAD domain that is bonded to a polypeptide binding moiety having a binding site that has binding specificity for a polypeptide that enhances serum half-life in vivo, wherein said drug composition has a longer in vivo serum half-life relative to said PLAD domain or functional variant of a PLAD domain, and has at least about 90% of the activity of the said PLAD domain or functional variant of a PLAD domain.

The invention relates to a conjugate or fusion protein comprising a PLAD domain or functional variant of a PLAD domain and a polypeptide that extends serum half-life in vivo. For example, serum albumin, an albumin fragment or albumin variant, or neonatal Fc receptor. In the conjugates, the PLAD domain or function variant of a PLAD domain and the polypeptide that extends serum half-life in vivo, can be conjugated directly or indirectly and covalently or noncovalently as described herein. In the fusion proteins, the PLAD domain or functional variant of a PLAD domain and the polypeptide that extends serum half-life in vivo can be present in single or multiple copies and in any desired orientation.

### BRIEF DESCRIPTION OF THE DRAWINGS

FIG. 1A is an alignment of the amino acid sequences of three V_{κ}s selected by binding to mouse serum albumin (MSA). The aligned amino acid sequences are from V_{κ}s designated MSA16, which is also referred to as DOM7m-16 (SEQ ID NO:1), MSA 12, which is also referred to as DOM7m-12 (SEQ ID NO:2), and MSA 26, which is also referred to as DOM7m-26 (SEQ ID NO:3).

FIG. 1B is an alignment of the amino acid sequences of six V_{κ}s selected by binding to rat serum albumin (RSA). The aligned amino acid sequences are from V_{κ}s designated DOM7r-1 (SEQ ID NO:4), DOM7r-3 (SEQ ID NO:5), DOM7r-4 (SEQ ID NO:6), DOM7r-5 (SEQ ID NO:7), DOM7r-7 (SEQ ID NO:8), and DOM7r-8 (SEQ ID NO:9).

FIG. 1C is an alignment of the amino acid sequences of six V_{κ}s selected by binding to human serum albumin (HSA). The aligned amino acid sequences are from V_{κ}s designated DOM7h-2 (SEQ ID NO:10), DOM7h-3 (SEQ ID NO:11), DOM7h-4 (SEQ ID NO:12), DOM7h-6 (SEQ ID NO: 13), DOM7h-1 (SEQ ID NO:14), and DOM7h-7 (SEQ ID NO:15).

FIG. 1D is an alignment of the amino acid sequences of seven V_{H}_{^{S}} selected by binding to human serum albumin and a consensus sequence (SEQ ID NO:23). The aligned sequences are from V_{H}_{^{S}} designated DOM7h-22 (SEQ ID NO:16), DOM7h-23 (SEQ ID NO:17), DOM7h-24 (SEQ ID NO:18), DOM7h-25 (SEQ ID NO:19), DOM7h-26 (SEQ ID NO:20), DOM7h-21 (SEQ ID NO:21), and DOM7h-27 (SEQ ID NO:22).

FIG. 1E is an alignment of the amino acid sequences of three V_{κ}s selected by binding to human serum albumin and rat serum albumin. The aligned amino acid sequences are from V_{κ}s designated DOM7h-8 (SEQ ID NO:24), DOM7r-13 (SEQ ID NO:25), and DOM7r-14 (SEQ ID NO:26).

FIGS. 2A and 2B are schematics maps of the vectors used to express the MSA16IL-1ra (also referred to as DOM7m-16/IL-1ra) and IL-1raMSA16 (also referred to as IL-1ra/DOM7m-16) fusions, respectively.

FIG. 2C-2D is an illustration of the nucleotide sequence (SEQ ID NO:27) encoding the IL-1raMSA16 fusion (also referred to as IL-1ra/DOM7m-16) and of the amino acid sequence (SEQ ID NO:28) of the fusion.

FIG. 2E-2F is an illustration of the nucleotide sequence (SEQ ID NO:29) encoding the MSA16IL-1ra fusion (also referred to as DOM7m-16/IL-1ra) and of the amino acid sequence (SEQ ID NO:30) of the fusion.

FIG. 2G-2H is an illustration of the nucleotide sequence (SEQ ID NO:31) encoding the DummyIL-1ra fusion that did not bind serum albumin, and of the amino acid sequence (SEQ ID NO:32) of the fusion.

FIG. 3A is an illustration showing that IL-1 induces the production of IL-8 by HeLa cells, and showing the mechanism by which IL-8 is detected in an ELISA assay.

FIG. 3B is a graph showing that IL-1ra (♦, labeled “R&D”), MSA16IL-1ra (▪) and IL-1raMSA16 (▴) each inhibited IL-1-induced secretion of IL-8 by cultured MRC-5 cells. The observed inhibition was dose dependent for IL-1ra, MSA16IL-1ra and IL-1 raMSA16.

FIGS. 4A-4C are graphs showing that IL-1ra (♦) and MSA16IL-1ra (▪) both inhibited IL-1-induced secretion of IL-8 by cultured MRC-5 cells in assays that included no mouse serum albumin (4A), 5% mouse serum albumin (4B) or 10% mouse serum albumin (4C). The observed inhibition was dose dependent for IL-1ra and MSA16IL-1ra under all conditions tested.

FIG. 5 is a schematic presentation of the results of an ELISA demonstrating that the MSA16IL-1ra fusion and the IL-1raMSA16 fusion both bound serum albumin, but the dummyIL1-ra fusion did not.

FIGS. 6A-6C are sensograms and tables showing BIACORE affinity data for clone DOM7h-1 binding to human serum albumin (HSA) (6A), DOM7h-7 binding to HSA (6B) and DOM7r-1 binding to rat serum albumin (RSA) (6C).

FIG. 7 is a table showing the affinities of DOM7-1, DOM7r-1, DOM7h-2, DOM7r-3, DOM7h-7, DOM7h-8, DOM7r-8, DOM7r-13, DOM7r-14, DOM7m-16, DOM7h-22, DOM7h-23, DOM7h-26, DOM7r-16, DOM7m-26, DOM7r-27 and DOM7R-31 for the serum albumins that they bind. DOM7h-8 also binds porcine serum albumin with and affinity (KD) of 60 nM.

FIG. 8A is an illustration of the nucleotide sequence (SEQ ED NO:33) of a nucleic acid encoding human interleukin 1 receptor antagonist (IL-1ra) deposited in GenBank under accession number NM_{—173842}. The nucleic acid has an open reading frame starting at position 65.

FIG. 8B is an illustration of the amino acid sequence of human IL-1ra (SEQ ED NO:34) encoded by the nucleic acid shown in FIG. 8A (SEQ ED NO:33). The mature protein consists of 152 amino acid residues (amino acid residues 26-177 of SEQ ED NO:34).

FIG. 9 is a graph showing the concentration (μg/mL) of MSA binding dAb/HA epitope tag fusion protein in mouse serum following a single intravenous (i.v.) injection (dose was about 1.5 mg/kg) into CD1 strain male animals over time (days). Serum concentration was determined by ELISA using goat anti-HA (Abeam, UK) capture and protein L-HRP (Invitrogen, USA) detection reagents. Standard curves of known concentrations of MSA binding dAb/HA fusion were set up in the presence of 1× mouse serum to ensure comparability with the test samples. Modelling with a 1 compartment model (WinNonlin Software, Pharsight Corp., USA) showed the MSA binding dAb/HA epitope tag fusion protein had a terminal phase t½ of 29.1 hours and an area under the curve of 559 hr·μg/mL.

FIG. 10 is an illustration of the amino acid sequences of V_{κ}s selected by binding to rat serum albumin (RSA). The illustrated sequences are from V_{κ}s designated DOM7r-15 (SEQ ID NO:37), DOM7r-16 (SEQ ID NO:38), DOM7r-17 (SEQ ID NO:39), DOM7r-18 (SEQ ID NO:40), DOM7r-19 (SEQ ID NO:41).

FIG. 11A-11B is an illustration of the amino acid sequences of the amino acid sequences of V_{H}_{^{S}} that bind rat serum albumin (RSA). The illustrated sequences are from V_{H}_{^{S}} designated DOM7r-20 (SEQ ID NO:42), DOM7r-21 (SEQ ID NO:43), DOM7r-22 (SEQ ID NO:44), DOM7r-23 (SEQ ID NO:45), DOM7r-24 (SEQ ID NO:46), DOM7r-25 (SEQ ID NO:47), DOM7r-26 (SEQ ID NO:48), DOM7r-27 (SEQ ID NO:49), DOM7r-28 (SEQ ID NO:50), DOM7r-29 (SEQ ID NO:51), DOM7r-30 (SEQ ID NO:52), DOM7r-31 (SEQ ID NO:53), DOM7r-32 (SEQ ID NO:54), and DOM7r-33 (SEQ ID NO:55).

FIG. 12 is a graph showing the concentration (% initial dose) of DOM7m-16, DOM7m-26 or a control dAb that does not bind MSA, each of which contained an HA epitope tag, in mouse serum following a single intravenous (i.v.) injection (dose was about 1.5 mg/kg) into CD1 strain male animals over time. Serum concentration was determined by ELISA using goat anti-HA (Abeam, UK) capture and protein L-HRP (Invitrogen, USA) detection reagents. Standard curves of known concentrations of MSA binding dAb/HA fusion were set up in the presence of 1× mouse serum to ensure comparability with the test samples. Modelling with a 1 compartment model (WinNonlin Software, Pharsight Corp., USA) showed control dAb had a terminal phase t½β of 20 minutes, while DOM7m-16, DOM7m-26 persisted in serum significantly longer.

FIG. 13 is a graph showing that DOM7m-16/IL-1ra was more effective than IL-1ra or ENBREL® (entarecept; Immunex Corporation) in treating arthritis in a mouse collagen-induced arthritis (CIA) model. Arthritis was induced and, beginning on day 21, mice were treated with Dexamethasone at 0.4 mg/Kg (Steroid), DOM7m-16/IL-1ra at 1 mg/Kg (IL-1ra/anti-SA 1 mg/kg) or 10 mg/Kg (IL-1ra/anti-SA 10 mg/kg), IL-1ra at 1 mg/Kg or 10 mg/Kg, ENBREL® (entarecept; Immunex Corporation) at 5 mg/Kg, or saline. The results show that DOM7m-16/IL-1ra was more effective than IL-1ra or ENBREL® (entarecept; Immunex Corporation) in this study. The response to IL-1ra was dose dependent, as expected, and that the response to DOM7m-16/IL-1 ra was also dose dependent. The average scores for treatment with DOM7m-16/IL-1ra at 1 mg/Kg were consistently lower than the average scores obtained by treatment with IL-1ra at 10 mg/kg, The results indicate that treatment with DOM7 nm-16/IL-1ra was 10 times more effective than IL-1ra in this study.

FIGS. 14A-14G illustrate the amino acid sequences of saporin polypeptides. FIG. 14A illustrates the amino acid sequence of saporin-2 precursor deposited as Swissprot Accession Number P27559 (SEQ ID NO:56). The signal peptide is amino acids 1-24 of SEQ ID NO:56. FIG. 14B illustrates the amino acid sequence of saporin-3 deposited as Swissprot Accession Number P27560 (SEQ ID NO:57). FIG. 14C illustrates the amino acid sequence of saporin-4 precursor deposited as Swissprot Accession Number P27561 (SEQ ID NO:58), The signal peptide is amino acids 1-24 of SEQ ID NO:58. FIG. 14D illustrates the amino acid sequence of saporin-5 deposited as Swissprot Accession Number Q41389 (SEQ ID NO:59). FIG. 14E illustrates the amino acid sequence of saporin-6 precursor deposited as Swissprot Accession Number P²⁰⁶⁵⁶ (SEQ ID NO:60). The signal peptide is amino acids 1-24 of SEQ ED NO:60, and a potential propeptide is amino acids 278-299 of SEQ ID NO:60. The mature polypeptide is amino acids 25-277 of SEQ ID NO:60 (SEQ ID NO:61), FIG. 14P illustrates the amino acid sequence of saporin-7 deposited as Swissprot Accession Number Q41391 (SEQ ID NO:62). FIG. 14G illustrates a consensus amino acid sequence encompassing several variants and isoforms of saporin-6 (SEQ ID NO:63).

FIG. 15 illustrates the amino acid sequences of several Camelid V_{HH}_{^{S}} that bind mouse serum albumin that are disclosed in WO 2004/041862. Sequence A (SEQ ID NO:68), Sequence B (SEQ ID NO:69), Sequence C (SEQ ID NO:70), Sequence D (SEQ ID NO:71), Sequence E (SEQ ID NO:72), Sequence F (SEQ ID NO:73), Sequence G (SEQ ID NO:74), Sequence H (SEQ ID NO:75), Sequence I (SEQ ID NO:76), Sequence J (SEQ ID NO:77), Sequence K (SEQ ID NO:78), Sequence L (SEQ ED NO:79), Sequence M (SEQ ED NO:80), Sequence N (SEQ ID NO:81), Sequence 0 (SEQ ID NO:82), Sequence P (SEQ ID NO:83), Sequence Q (SEQ ID NO:84).

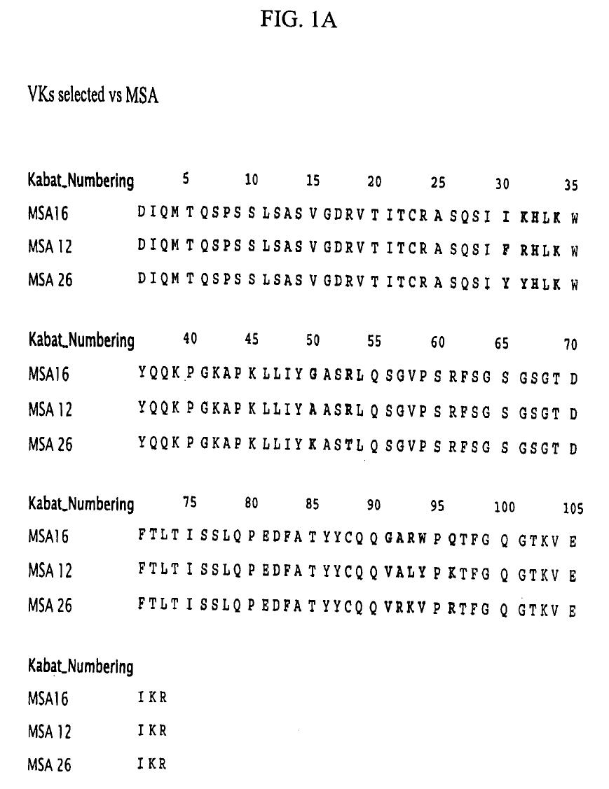

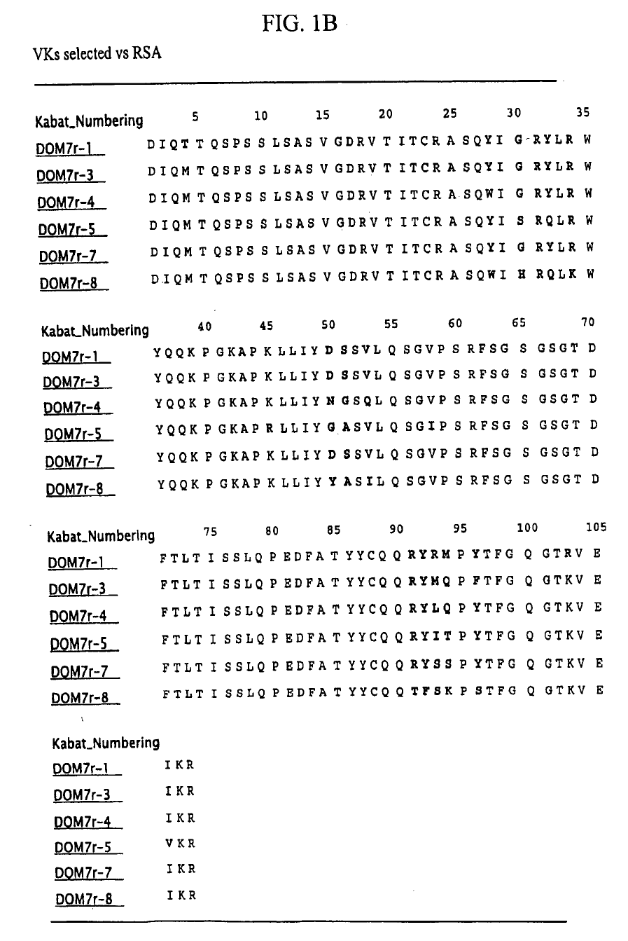

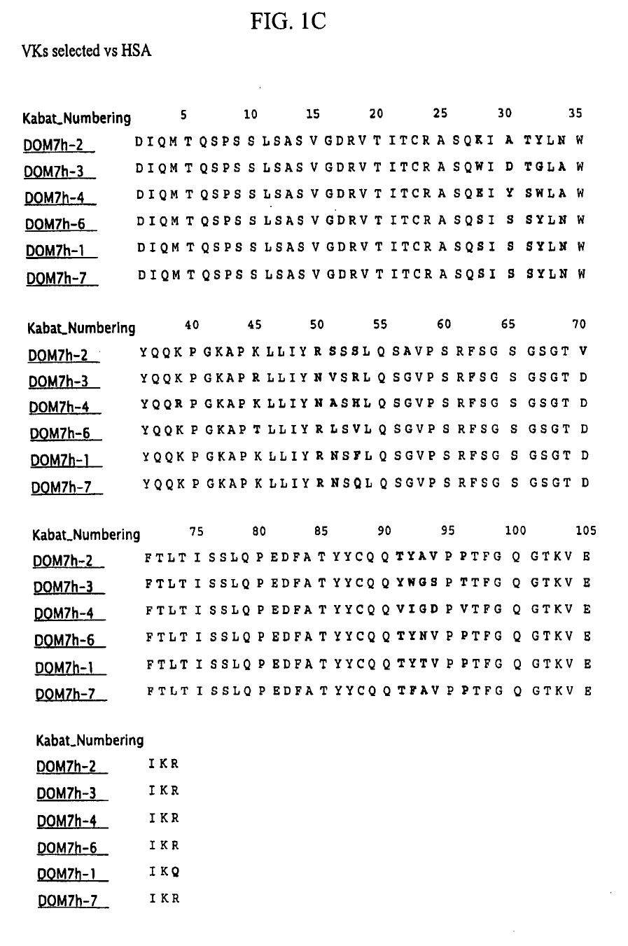

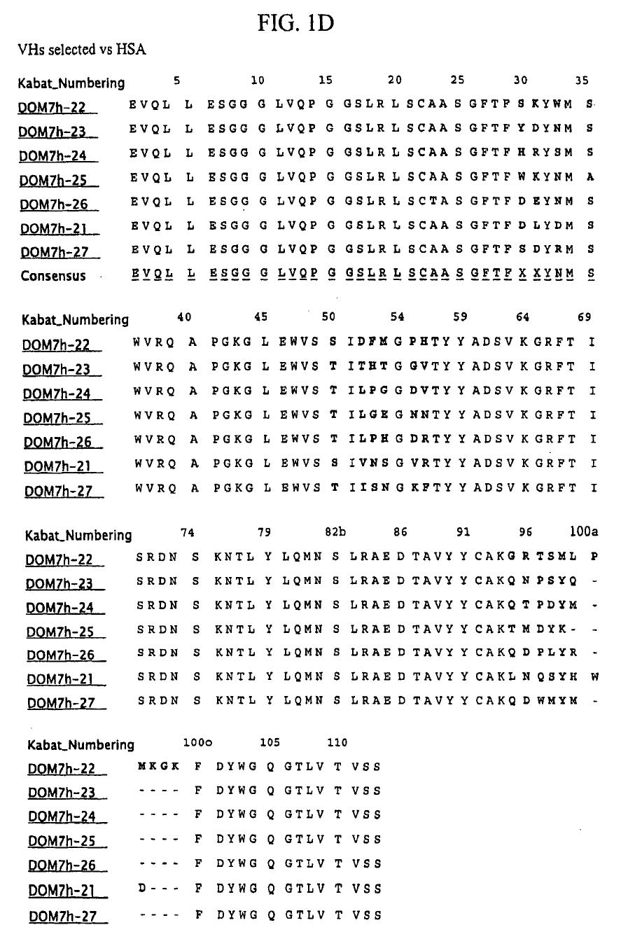

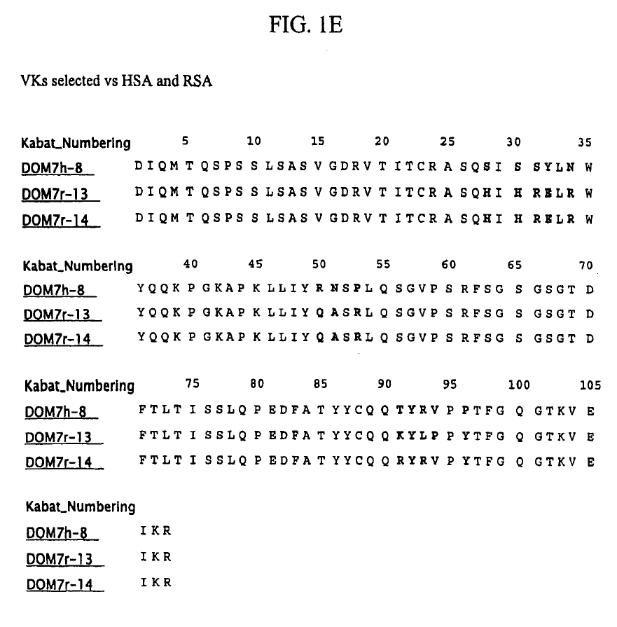

### DETAILED DESCRIPTION OF THE INVENTION

Within this specification embodiments have been described in a way which enables a clear and concise specification to be written, but it is intended and will be appreciated that embodiments may be variously combined or separated without parting from the invention.

Known compositions of matter having a structural formula identical to any one of the embodiments of the invention are explicitly disclaimed per se. In contrast, novel compositions of matter, novel combinations of the known compositions, novel uses of the known compositions or novel methods involving the known compositions are not disclaimed.

As used herein, “drug” refers to any compound (e.g., small organic molecule, nucleic acid, polypeptide) that can be administered to an individual to produce a beneficial therapeutic or diagnostic effect though binding to and/or altering the function of a biological target molecule in the individual. The target molecule can be an endogenous target molecule encoded by the individual's genome (e.g., an enzyme, receptor, growth factor, cytokine encoded by the individual's genome) or an exogenous target molecule encoded by the genome of a pathogen (e.g., an enzyme encoded by the genome of a virus, bacterium, fungus, nematode or other pathogen).

As used herein, “drug composition” refers to a composition comprising a drug that is covalently or noncovalently bonded to a polypeptide binding moiety, wherein the polypeptide binding moiety contains a binding site (e.g., an antigen-binding site) that has binding specificity for a polypeptide that enhances serum half-life in vivo. The drug composition can be a conjugate wherein the drug is covalently or noncovalently bonded to the polypeptide binding moiety. The drug can be covalently or noncovalently bonded to the polypeptide binding moiety directly or indirectly (e.g. through a suitable linker and/or noncovalent binding of complementary binding partners (e.g., biotin and avidin)). When complementary binding partners are employed, one of the binding partners can be covalently bonded to the drug directly or through a suitable linker moiety, and the complementary binding partner can be covalently bonded to the polypeptide binding moiety directly or through a suitable linker moiety. When the drug is a polypeptide or peptide, the drug composition can be a fusion protein, wherein the polypeptide or peptide drug and the polypeptide binding moiety are discrete parts (moieties) of a continuous polypeptide chain.

As used herein “conjugate” refers to a composition comprising an antigen-binding fragment of an antibody that binds serum albumin that is bonded to a drug. Such conjugates include “drug conjugates,” which comprise an antigen-binding fragment of an antibody that binds serum albumin to which a drug is covalently bonded, and “noncovlaent drug conjugates,” which comprise an antigen-binding fragment of an antibody that binds serum albumin to which a drug is noncovalently bonded.

As used herein, “drug conjugate” refers to a composition comprising an antigen-binding fragment of an antibody that binds serum albumin to which a drug is covalently bonded. The drug can be covalently bonded to the antigen-binding fragment directly or indirectly through a suitable linker moiety. The drug can be bonded to the antigen-binding fragment at any suitable position, such as the amino-terminus, the carboxyl-terminus or through suitable amino acid side chains (e.g., the ε amino group of lysine).

As used herein, “noncovalent drug conjugate” refers to a composition comprising an antigen-binding fragment of an antibody that binds serum albumin to which a drug is noncovalently bonded. The drug can be noncovalently bonded to the antigen-binding fragment directly (e.g., electrostatic interaction, hydrophobic interaction) or indirectly (e.g., through noncovalent binding of complementary binding partners (e.g., biotin and avidin), wherein one partner is covalently bonded to drug and the complementary binding partner is covalently bonded to the antigen-binding fragment). When complementary binding partners are employed, one of the binding partners can be covalently bonded to the drug directly or through a suitable linker moiety, and the complementary binding partner can be covalently bonded to the antigen-binding fragment of an antibody that binds serum albumin directly or through a suitable linker moiety.

As used herein, “drug fusion” refers to a fusion protein that comprises an antigen-binding fragment of an antibody that binds serum albumin and a polypeptide drug. The antigen-binding fragment of an antibody that binds serum albumin and the polypeptide drug are present as discrete parts (moieties) of a single continuous polypeptide chain.

As used herein the term “drug basis” refers to activities of drug compositions and drugs that are normalized based on the amount of drug (or drug moiety) used to assess, measure or determine activity. Generally, the drug compositions of the invention (e.g., drug conjugate, noncovalent drug conjugate, drug fusion) have a larger molecular weight than the drug they contain. Thus, equivalent amounts of drug composition and drug, by weight, will contain different amounts of drug on a molecular or molar basis. For example, if a drug composition of the invention has a molecular weight that is twice the molecular weight of the drug it comprises, activities can be determined on a “drug basis” using 2 μg of drug composition and 1 μg of drug, because these quantities would contain the same amount of drug (as free drug or as part of the drug composition). Activities can be normalized and expressed on a “drug basis” using appropriate calculations, for example, by expressing activity on a per target binding site basis or, for enzyme drugs, on a per active site basis.

As used herein “interleukin 1 receptor antagonist” (IL-1ra) refers to naturally occurring or endogenous mammalian IL-1ra proteins and to proteins having an amino acid sequence which is the same as that of a naturally occurring or endogenous corresponding mammalian IL-1ra protein (e.g., recombinant proteins, synthetic proteins (i.e., produced using the methods of synthetic organic chemistry)). Accordingly, as defined herein, the term includes mature protein, polymorphic or allelic variants, and other isoforms of a IL-1ra (e.g., produced by alternative splicing or other cellular processes), and modified or unmodified forms of the foregoing (e.g., lipidated, glycosylated, PEGylated). Naturally occurring or endogenous IL-1ra include wild type proteins such as mature IL-1ra, polymorphic or allelic variants and other isoforms which occur naturally in mammals (e.g., humans, non-human primates). Such proteins can be recovered or isolated from a source which naturally produces IL-1ra, for example. These proteins and IL-1ra proteins having the same amino acid sequence as a naturally occurring or endogenous corresponding IL-1ra, are referred to by the name of the corresponding mammal. For example, where the corresponding mammal is a human, the protein is designated as a human IL-1ra.

“Functional variants” of IL-1ra include functional fragments, functional mutant proteins, and/or functional fusion proteins which can be produce using suitable methods (e.g., mutagenesis (e.g., chemical mutagenesis, radiation mutagenesis), recombinant DNA techniques). A “functional variant” antagonizes interleukin-1 type 1 receptor. Generally, fragments or portions of IL-1ra include those having a deletion and/or addition (i.e., one or more amino acid deletions and/or additions) of an amino acid (i.e., one or more amino acids) relative to the mature IL-1ra (such as N-terminal, C-terminal or internal deletions). Fragments or portions in which only contiguous amino acids have been deleted or in which non-contiguous amino acids have been deleted relative to mature IL-1ra are also envisioned.

A functional variant of human IL-1ra can have at least about 80%, or at least about 85%, or at least about 90%, or at least about 95%, or at least about 96%, or at least about 97%, or at least about 98%, or at least about 99% amino acid sequence identity with the mature 152 amino acid form of human IL-1ra and antagonize human Interleukin-1 type 1 receptor. (See, Eisenberg et al, Nature 343:341-346 (1990).) The variant can comprise one or more additional amino acids (e.g., comprise 153 or 154 or more amino acids). For example, the variant IL-1ra can have an amino acid sequence that consists of an amino-terminal methionine residue followed by residues 26 to 177 of SEQ ID NO:33. (KINERET® (anakinra), Amgen Inc.).

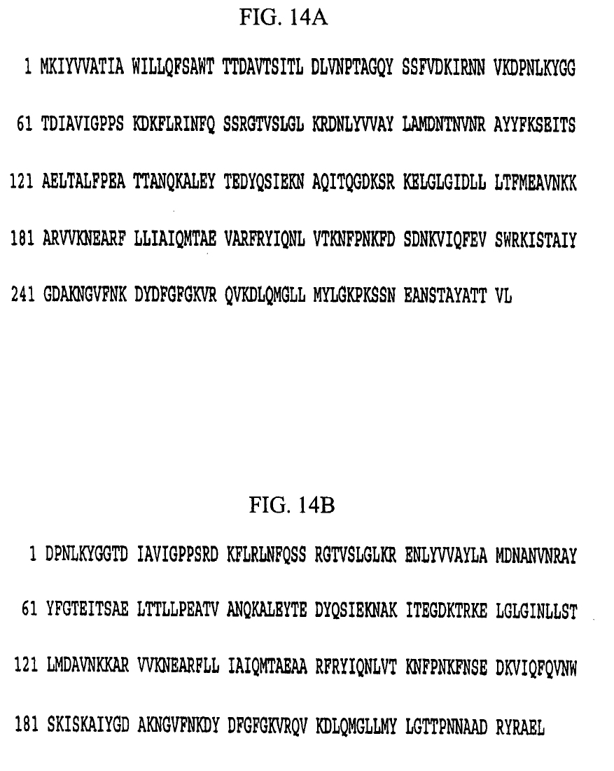

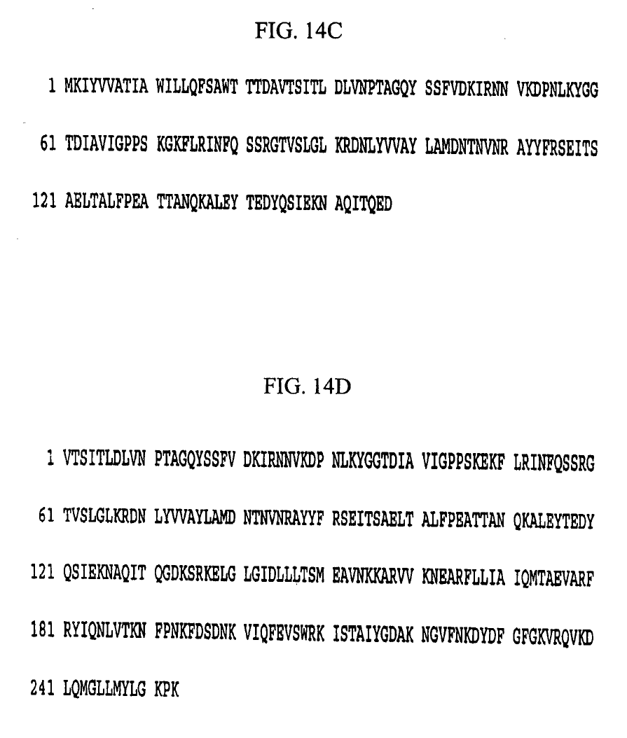

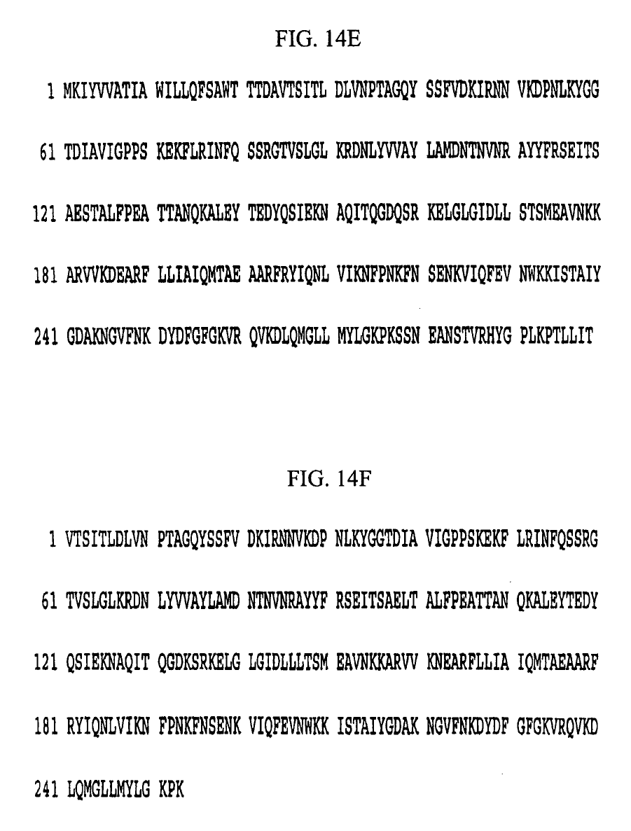

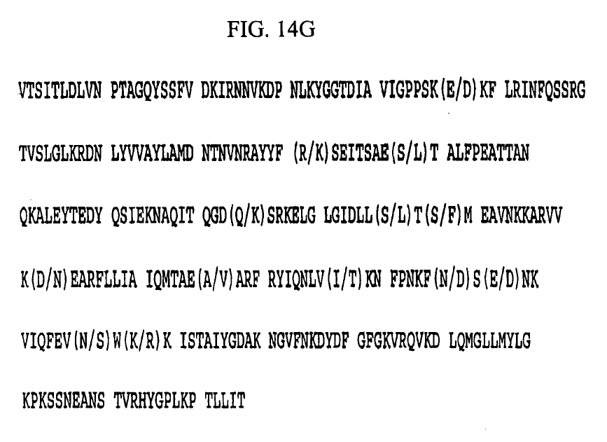

As used herein “saporin” refers to a family of single-chain ribosome-inactivating polypeptides produced by the plant Saponaria officinalis. (Stirpe, F., et al., Biochem. J. 216:617-625 (1983), Bagga, S. et al., J. Biol. Chem. 278:4813-4820 (2003).) Saporin polypeptides exist is several forms that differ in length and/or amino acid sequence. (See, e.g., Id. and Barthelemy, I. et al., J. Biol. Chem. 268:6541-6548 (1993).) Saporin-6 is the most active form of saporin. (Bagga, S. et al, J. Biol. Chem. 278:4813-4820 (2003).) At least four naturally occurring isoforms of saporin-6 in which the amino acid at position 48 of the mature polypeptide (SEQ ID NO:61) is Asp or Glu, and the amino acid a position 91 of the mature polypeptide (SEQ ID NO:61) is Arg or Lys have been described. (Barthelemy, I et al., J. Biol. Chem. 268:6541-6548 (1993).) Additional forms of saporin-6 include polypeptides in which the amino acid at position 99 of the mature polypeptide (SEQ ID NO:61) is Set or Leu; the amino acid at position 134 of the mature polypeptide (SEQ ID NO:61) is Gln or Lys; the amino acid at position 147 of the mature polypeptide (SEQ ID NO:61) is Ser or Leu; the amino acid at position 149 of the mature polypeptide (SEQ ID NO:61) is Ser or Phe; the amino acid at position 162 of the mature polypeptide (SEQ ID NO:61) is Asp or Asn; the amino acid at position 177 of the mature polypeptide (SEQ ID NO:61) is Ala or Val; the amino acid at position 188 of the mature polypeptide (SEQ ID NO:61) is Ile or Thr; the amino acid at position 196 of the mature polypeptide (SEQ ID NO:61) is Asn or Asp; the amino acid at position 198 of the mature polypeptide (SEQ ID NO:61) is Glu or Asp; the amino acid at position 231 of the mature polypeptide (SEQ ID NO:61) is Asn or Ser; and polypeptides in which the amino acid at position 233 of the mature polypeptide (SEQ ID NO:61) is Lys or Arg. (Id.) A consensus sequence encompassing these isoforms and variants is presented in FIG. 14G (SEQ ID NO:63).

Accordingly the term “saporin” includes precursor protein, mature polypeptide, native protein, polymorphic or allelic variants, and other isoforms (e.g., produced by alternative splicing or other cellular processes), and modified or unmodified forms of the foregoing (e.g., lipidated, glycosylated, PEGylated) including naturally occurring, synthetic or recombinantly produced polypeptides. Naturally occurring or endogenous saporin include wild type proteins such as mature saporin (e.g., mature saporin-6), polymorphic or allelic variants and other isoforms which occur naturally in Saponaria officinalis. Such proteins can be recovered or isolated from Saponaria officinalis using any suitable methods, “Functional Variants” of saporin include functional fragments, functional mutant proteins, and/or functional fusion proteins which can be produced using suitable methods (e.g., mutagenesis (e.g., chemical mutagenesis, radiation mutagenesis), recombinant DNA techniques). Generally, fragments or portions of saporin (e.g., saporin-6) include those having a deletion and/or addition (i.e., one or more amino acid deletions and/or additions) of an amino acid (i.e., one or more amino acids) relative to mature saporin (such as N-terminal, C-terminal or internal deletions). Fragments or portions in which only contiguous amino acids have been deleted or in which non-contiguous amino acids have been deleted relative to mature saporin are also envisioned. A variety of functional variants of saporin can be prepared. For example, fusion proteins of saporin-6 that contain amino-terminal extensions have been prepared and shown to retain full ribosome-inhibiting activity in rabbit reticulocyte lysate assays. (Barthelemy, I et al., J. Biol. Chem. 268:6541-6548 (1993).) Variants or saporin-6 is which an active site residue, Tyr72, Tyr120, Glu176, Arg 179 ox Trp208 (amino acids 72, 120, 176, 179 or 208 of SEQ ID NO:61), was replaced with alanine had reduced cytotoxic activity in in vitro assays. (Bagga, S. et al, J, Biol. Chem. 278:4813-4820 (2003).) Accordingly, if preparing additional functional variants of saporin is desired, mutation, substitution, replacement, deletion or modification of the active site residues should be avoided. Preferably, a functional variant of saporin that contains fewer amino acids than naturally occurring mature polypeptide includes at least the active site. For example, a variant of saporin-6 that contains fewer amino acids than naturally occurring mature saporin-6 can include the active site residues of mature saporin-6 (Tyr72, Tyr120, Glu176, Arg 179 and Trp208 (amino acids 72, 120, 176, 179 and 208 of SEQ ID NO:61)), and be at least about 137 amino acids in length, at least about 150 amino acids in length, at least about 175 amino acids in length, at least about 200 amino acids in length, at least about 225 amino acids in length or at least about 250 amino acids in length.

A “functional variant” of saporin has ribosome-inactivating activity (e.g., rRNA N-Glycosidase activity) and/or cytotoxic activity. Such activity can readily be assessed using any suitable method, such as inhibition of protein synthesis using the well-known rabbit reticulocyte lysate assay or any of the well-known cytotoxicity assays that employ tumor cell lines, (See, e.g., Bagga, S. et al, J. Biol. Chem. 278:4813-4820 (2003) and Barthelemy, I. et al., J. Biol. Chem. 268:6541-6548 (1993).)

In some embodiments, a functional variant of saporin has at least about 80%, or at least about 85%, or at least about 90% or at least about 91%, or at least about 92%, or at least about 93%, or at least about 94%, or at least about 95%, or at least about 96%, or at least about 97%, or at least about 98%, or at least about 99% amino acid sequence identity with mature saporin-6 (SEQ ID NO: 61).

The invention relates to drug compositions that comprise a drug and a polypeptide binding moiety that contains a binding site (e.g., an antigen-binding site) that has binding specificity for a polypeptide that enhances serum half-life in viva. As described herein in detail with respect to drug compositions that comprise an antigen-binding fragment of an antibody that has binding specificity for serum alburnin, the drug and the polypeptide binding moiety can be bonded to each other covalently or noncovalently. In some embodiments, the drug composition is a fusion protein that comprises a polypeptide drug and a polypeptide binding moiety that contains an antigen-binding site that has binding specificity for a polypeptide that enhances serum half-life in vivo. In other embodiments, the drug composition comprises a drug that is covalently or noncovalently bonded to a polypeptide binding moiety that contains an antigen-binding site that has binding specificity for a polypeptide that enhances serum half-life in vivo.

Typically, a polypeptide that enhances serum half-life in vivo is a polypeptide which occurs naturally in vivo and which resists degradation or removal by endogenous mechanisms which remove unwanted material from the organism (e.g., human). For example, a polypeptide that enhances serum half-life in vivo can be selected from proteins from the extracellular matrix, proteins found in blood, proteins found at the blood brain barrier or in neural tissue, proteins localised to the kidney, liver, lung, heart, skin or bone, stress proteins, disease-specific proteins, or proteins involved in Fc transport.

Suitable polypeptides that enhance serum half-life in vivo include, far example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g., soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g., fibrin, α-2 macroglobulin, serum albumin, fibrinogen (e.g., fibrinogen A, fibrinogen B), serum amyloid protein A, haptoglobin, profilin, ubiquitin, uteroglobulin and β-2-microglobulin), enzymes and enzyme inhibitors (e.g., plasminogen, lysozyme, cystatin C, alpha-1-antitrypsin and pancreatic trypsin inhibitor), proteins of the immune system, such as immunoglobulin proteins (e.g., IgA, IgD, IgE, IgG, IgM, immunoglobulin light chains (kappa/lambda)), transport proteins (e.g., retinol binding protein, α-1 microglobulin), defensins (e.g., beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhances serum half-life in vivo also include proteins localized to the kidney (e.g., polycystin, type IV collagen, organic anion transporter K1, Heymann's antigen), proteins localized to the liver (e.g., alcohol dehydrogenase, G250), proteins localized to the lung (e.g., secretory component, which binds IgA), proteins localized to the heart (e.g., HSP 27, which is associated with dilated cardiomyopathy), proteins localized to the skin (e.g., keratin), bone specific proteins such as morphogenic proteins (BMPs), which are a subset of the transforming growth factor β superfamily of proteins that demonstrate osteogenic activity (e.g., BMP-2, BMP-4, BMP-5, BMP-6, BMP-7, BMP-8), tumor specific proteins (e.g., trophoblast antigen, herceptin receptor, oestrogen receptor, cathepsins (e.g., cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

Examples of suitable albumin, albumin fragments or albumin variants for use in the invention are described in WO 2005/077042A2, which is incorporated herein by reference in its entirety. In particular, the following albumin, albumin fragments or albumin variants can be used in the present invention:
SEQ ID NO:1 (as disclosed in WO 2005/077042A2, this sequence being explicitly incorporated into the present disclosure by reference);
Albumin fragment or variant comprising or consisting of amino acids 1-387 of SEQ ED NO:1 in WO 2005/077042A2;
Albumin, or fragment or variant thereof, comprising an amino acid sequence selected from the group consisting of: (a) amino acids 54 to 61 of SEQ ED NO:1 in WO 2005/077042A2; (b) amino acids 76 to 89 of SEQ ID NO:1 in WO 2005/077042A2; (c) amino acids 92 to 100 of SEQ ED NO:1 in WO 2005/077042A2; (d) amino acids 170 to 176 of SEQ ID NO:1 in WO 2005/077042A2; (e) amino acids 247 to 252 of SEQ ID NO:1 in WO 2005/077042A2; (f) amino acids 266 to 277 of SEQ ED NO:1 in WO 2005/077042A2; (g) amino acids 280 to 288 of SEQ ED NO:1 in WO 2005/077042A2; (h) amino acids 362 to 368 of SEQ ED NO:1 in WO 2005/077042A2; (i) amino acids 439 to 447 of SEQ ED NO:1 in WO 2005/077042A2 (j) amino acids 462 to 475 of SEQ ED NO:1 in WO 2005/077042A2; (k) amino acids 478 to 486 of SEQ ID NO:1 in WO 2005/077042A2; and (1) amino acids 560 to 566 of SEQ ED NO:1 in WO 2005/077042A2.

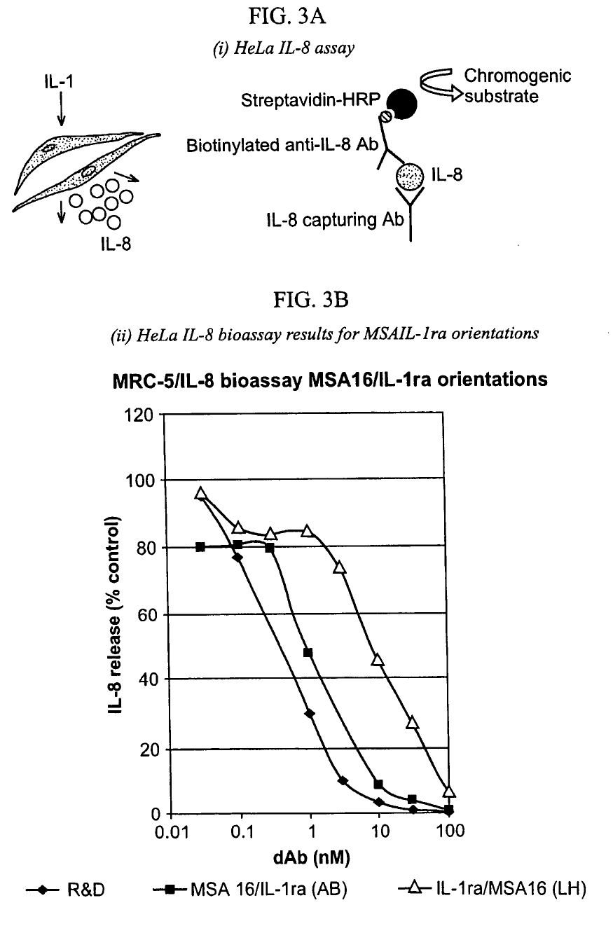

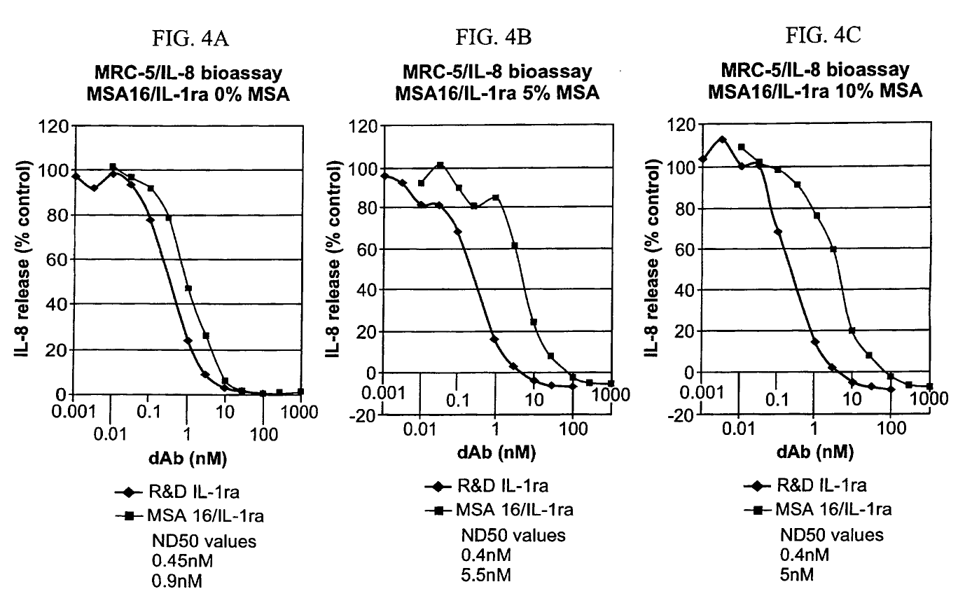

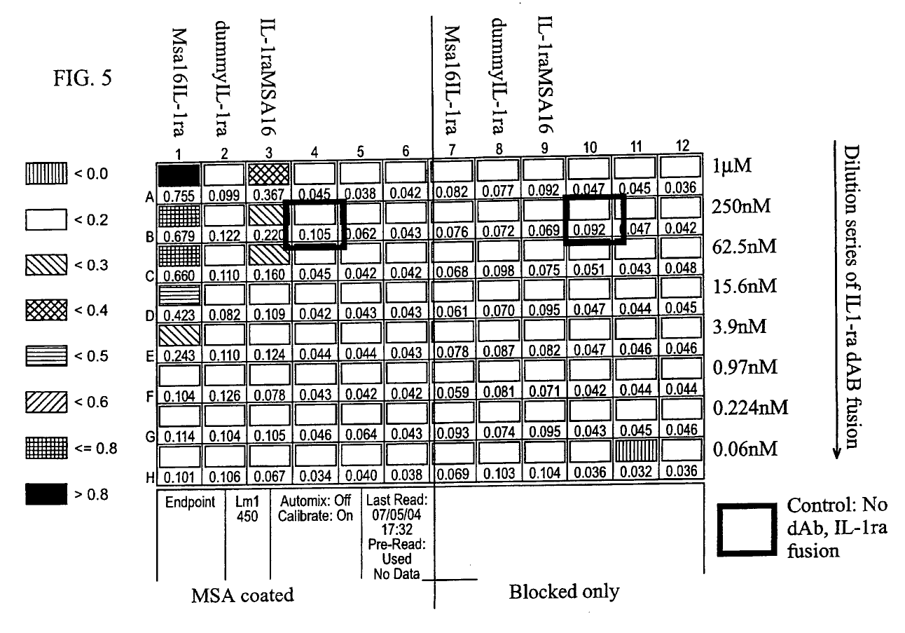

Further examples of suitable albumin, fragments and analogs for use in a TNFR1-binding ligand according to the invention are described in WO 03/076567A2, which is incorporated herein by reference in its entirety. In particular, the following albumin, fragments or variants can be used in the present invention:
Human serum albumin as described in WO 03/076567A2, eg, in FIG. 3 (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al., FEBS Letters 55:136 (1975); Behrens, et al, Fed. Proc. 34:591 (1975); Lawn, et al, Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, eg, HA(1-373., HA(1-388), HA(1-389), HA(1-369), and HA(1-419) and fragments between 1-369 and 1-419;
An albumin fragment or variant as described in EP 399666, eg, HA(1-177) and HA(1-200) and fragments between HA(1-X), where X is any number from 178 to 199.

The drug compositions of the invention can comprise any polypeptide binding moiety that contains a binding site (e.g., an antigen-binding site) that has binding specificity for a polypeptide that enhances serum half-life in vivo. Preferably, the polypeptide binding moiety comprises at least 31, at least about 40, at least about 50, at least about 60, at least about 70, at least about 80 amino acids, at least about 90-amino acids, at least about 100 amino acids or at lease about 110 amino acids as a separate molecular entity. Preferably, the polypeptide binding moiety binds a polypeptide that enhances serum half-life in vivo with a KD of at least about 5 mM KD (KD=K_{off}(kd)/Kₒₙ (ka)). In some embodiments, the polypeptide binding moiety binds a polypeptide that enhances serum half-life in vivo with a KD of about 10 to about 100 nM, or about 100 nM to about 500 nM, or about 500 nM to about 5 mM, as determined by surface plasmon resonance (e.g., using a BIACORE instrument). In particular embodiments, the polypeptide binding moiety binds a polypeptide that enhances serum half-life in vivo with a KD of about 50 nM, or about 70 nM, or about 100 nM, or about 150 nM or about 200 nM.

Preferably, the polypeptide binding moiety that contains a binding site (e.g., an antigen-binding site) that has binding specificity for a polypeptide that enhances serum half-life in vivo is not a prokaryotic or bacterial polypeptide or peptide. Preferably, the polypeptide binding moiety is a eukaryotic, mammalian or human polypeptide or peptide.

In certain embodiments, the polypeptide binding moiety that contains a binding site (e.g., an antigen-binding site) that has binding specificity for a polypeptide that enhances serum half-life in vivo is a folded protein domain. In other embodiments, the polypeptide binding moiety has a molecular weight of at least about 4 KDa, at least about 4.5 KDa, at least about 5 KDa, at least about 5.5 KDa, at least about 6 KDa, at least about 6.5 KDa, at least about 7 KDa, at least about 7.5 KDa or at least about 8 KDa as a separate molecular entity.

Suitable polypeptide binding moieties that contain a binding site (e.g., an antigen-binding site) that has binding specificity for a polypeptide that enhances serum half-life in vivo can be identified using any suitable method, such as by screening naturally occurring or non-naturally occurring polypeptides in a suitable adhesion assay. As described herein, preferred polypeptide binding moieties that have an antigen-binding site for a polypeptide that enhances serum half-life in vivo are antigen-binding fragments of antibodies that have binding specificity for serum albumin. However, antigen-binding fragments of antibodies that have binding specificity for other polypeptides that enhance serum half-life in vivo can be used in the invention.

If desired, one or more of the complementarity determining regions (CDRs) of an antibody or antigen-binding fragment thereof that binds a polypeptide that enhances serum half-life in vivo can be formatted into a non-immunoglobulin structure that retains the antigen-binding specificity of the antibody or antigen-binding fragment. The drug compositions of the invention can comprise such a non-immunoglobulin binding moiety. Such non-immunoglobulin binding moieties can be prepared using any suitable method, for example natural bacterial receptors such as SpA have been used as scaffolds for the grafting of CDRs to generate polypeptide binding moieties which specifically bind an epitope. Details of this procedure are described in U.S. Pat. No. 5,831,012, the teachings of which are incorporated herein by reference. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. 310:591-601 (2001), and scaffolds such as those described in WO 00/69907 (Medical Research Council), which are based for example on the ring structure of bacterial GroEL or other chaperone polypeptides.

In some embodiments, the drug composition of the invention comprises a non-immunoglobulin binding moiety that has binding specificity for serum albumin, wherein the non-immunoglobulin binding moiety comprises one, two or three of the CDRs of a V_{H}, V_{κ} or V_{HH} described herein and a suitable scaffold. In certain embodiments, the non-immunoglobulin binding moiety comprises CDR3 but not CDR1 or CDR2 of a V_{H}, V_{κ} or V_{HH} described herein and a suitable scaffold. In other embodiments, the non-immunoglobulin binding moiety comprises CDR1 and CDR2, but not CDR3 of a V_{H}, V_{κ} or V_{HH} described herein and a suitable scaffold. In other embodiments, the non-immunoglobulin binding moiety comprises CDR1, CDR2 and CDR3 of a V_{H}, V_{κ} or V_{HH} described herein and a suitable scaffold. In other embodiments, the drug composition comprises only CDR3 of a V_{H}, V_{κ} or V_{HH} described herein and a drug.

The drug compositions of the invention can be prepared using suitable methods, such as the methods described herein for preparation of drug fusions, drug conjugates and noncovalent drug conjugates. Additionally, the drug compositions of the invention have the advantages and the utilities that are described in detail herein with respect to drug fusions, drug conjugates and noncovalent drug conjugates.

The invention provides drug compositions (e.g., drug conjugates, noncovalent drug conjugates, drug fusions) that have improved pharmacokinetic properties (e.g., increase serum half-life) and other advantages in comparison to the drug alone (unconjugated drug, unfused drug). The drug conjugates, noncovalent drug conjugates and drug fusions comprise an antigen-binding fragment of an antibody that has binding specificity for serum albumin and one or more desired drugs.

As described herein, drug compositions (e.g., drug conjugates, noncovalent drug conjugates, drug fusions) of the invention can have dramatically prolonged in vivo serum half-life and/or increased AUC, as compared to drug alone. In addition, the activity of the drug is generally not substantially altered in the drug composition (e.g., drug conjugate, noncovalent drug conjugate, drug fusion). However, some change in the activity of a drug composition compared to drug alone is acceptable and is generally compensated for by the improved pharmacokinetic properties of the drug composition (e.g., drug conjugate, noncovalent drug conjugate, drug fusion). For example, drug compositions (e.g., drug conjugates, noncovalent drug conjugates, drug fusions) may bind the drug target with lower affinity than drug alone, but have about equivalent or superior efficacy in comparison to drug alone due to the improved pharmacokinetic properties (e.g., prolonged in vivo serum half-life, larger AUC) of the drug composition. In addition, lower amounts of drug compositions (e.g., drug conjugates, noncovalent drug conjugates and drug fusions) can be administed to achieve the desired therapeutic or diagnostic effect. Preferably the activity of the drug composition (e.g., drug conjugate, noncovalent drug conjugate, drug fusion) differs from that of the drug alone by a factor of no more than about 100, or no more than about 50, or no more than about 10, or no more than about 5, or no more than about 4, or no more than about 3, or no more than about 2. For example, a drug can have a KD, Ki or neutralizing dose 50 (ND50) of 1 nM, and a drug composition (e.g., drug conjugate, noncovalent drug conjugate, drug fusion) can have a KD, Ki or ND50 of about 2 nM, or about 3 nM, or about 4 nM, or about 5 nM, or about 10 nM.

Preferably, the activity of the drug composition (e.g., drug conjugate, noncovalent drug conjugate, drug fusion) is not substantially reduced as compared to the activity of the drug. In certain embodiments, the activity of the drug composition is reduced, relative to the activity of drug, by no more than about 10%, no more than about 9%, no more than about 8%, no more than about 7%, no more than about 6%, no more than about 5%, no more than about 4%, no more than about 3%, no more than about 2%, no more than about 1% or is substantially unchanged. Alternatively stated, the drug composition (e.g., drug conjugate, noncovalent drug conjugate, drug fusion) retains at least about 90%, at least about 91%, at least about 92%, at least about 93%, at least about 94%, at least about 95%, at least about 96%, at least about 97%, at least about 98%, at least about 99% of the activity of the drug, or substantially the same activity as the drug. Preferably, the activity of drug compositions (e.g., drug conjugate, noncovalent drug conjugate, drug fusion) and drugs are determined and/or compared on a “drug basis.”

As described and shown herein, the drug compositions (e.g., drug conjugate, noncovalent drug conjugate, drug fusion) of the invention can have greater activity (e.g., in vivo activity) than drug alone. For example, as shown in Example 6, DOM7m-16/IL-1ra was more effective in treating arthritis in a mouse model than IL-1ra when these agents were administered at the same dose by weight (10 mg/Kg or 1 mg/Kg). DOM7m-16/IL-1ra was more effective even though its molecular weight is approximately twice the molecular weight of IL-1ra. Thus, mice that received DOM7m-16/IL-1ra received only about half of the IL-1ra (as a moiety in DOM7m-16/IL1-ra) as mice that received IL-1ra.

In certain embodiments, the drug composition (e.g., drug conjugate, noncovalent drug conjugate, drug fusion) has greater activity (e.g., in vivo activity) than drug, for example, the drug composition can have at least about 100%, at least about 150%, at least about 200%, at least about 250%, at least about 300%, at least about 350%, at least about 400%, at least about 450%, or at least about 500% of the activity of drug. Preferably, the activity of drug compositions (e.g., drug conjugate, noncovalent drug conjugate, drug fusion) and drugs are determined and/or compared on a “drug basis.” The activity of drug compositions (e.g., drug conjugate, noncovalent drug conjugate, drug fusion) and drugs can be determined using a suitable in vitro or in vivo system. In certain embodiments, a drug composition (e.g., drug conjugate, noncovalent drug conjugate, drug fusion) has greater activity than the drug it comprises, as determined in vivo. In other embodiments, a drug composition (e.g., drug conjugate, noncovalent drug conjugate, drug fusion) has greater activity than the drug it comprises, as determined in vitro.

Drug compositions (e.g., drug conjugates, noncovalent drug conjugates, drug fusions) that comprise a domain antibody (dAb) that has binding specificity for serum albumin provide further advantages. Domain antibodies are very stable, are small relative to antibodies and other antigen-binding fragments of antibodies, can be produced in high yields by expression in E. coli or yeast (e.g., Pichia pastoris), and as described herein antigen-binding fragments of antibodies that bind serum albumin can be easily selected from libraries of human origin or from any desired species. Accordingly, drug compositions (e.g., drug conjugates, noncovalent drug conjugates, drug fusions) that comprise a dAb that binds serum albumin can be produced more easily than therapeutics that are generally produced in mammalian cells (e.g., human, humanized or chimeric antibodies) and dAbs that are not immunogenic can be used (e.g., a human dAb can be used for a drug fusion or drug conjugate for treating or diagnosing disease in humans).

The immunogenicity of a drug can be reduced when the drug is part of a drug composition (e.g., drug conjugate, noncovalent drug conjugate, drug fusion) that contains a polypeptide binding moiety that binds serum albumin (e.g., an antigen-binding fragment of an antibody that binds serum albumin). Accordingly, a drug can be less immunogenic (than drug alone) or be substantially non-immunogenic in the context of a drug composition that contains a polypeptide binding moiety that binds serum albumin (e.g., drug conjugate, noncovalent drug conjugate, drug fusion). Thus, such drug compositions (e.g., drug conjugates, noncovalent drug conjugates, drug fusions) can be administered to a subject repeatedly over time with minimal loss of efficacy due to the elaboration of anti-drug antibodies by the subject's immune system.

Additionally, the drug compositions (e.g., drug conjugates, noncovalent drug conjugates, drug fusions) described herein can have an enhanced safety profile and fewer side effects than drug alone. For example, as a result of the serum albumin-binding activity of the antigen-binding fragment of an antibody that has binding specificity for serum albumin, the drug fusions and conjugates (drug conjugate, noncovalent drug conjugate) have enhanced residence time in the vascular circulation. Additionally, the conjugates and drug fusions are substantially unable to cross the blood brain barrier and to accumulate in the central nervous system following systemic administration (e.g., intravascular administration). Accordingly, conjugates (drug conjugate, noncovalent drug conjugate) and drug fusions that contain a drug that has neurological toxicity or undesirable psychotropic effects can be administered with greater safety and reduced side effects in comparison to the drug alone. Similarly, the conjugates (drug conjugate, noncovalent drug conjugate) and drug fusions can have reduced toxicity toward particular organs (e.g., kidney or liver) than drug alone. The conjugates and drug fusions described herein can also be used to sequester a drug or a target that binds a drug (e.g. a toxin) in the vascular circulation, thereby decreasing the effects of the drug or target on tissues (e.g., inhibiting the effects of a toxin).

Suitable methods for pharmacokinetic analysis and determination of in vivo half-life are well known in the art. Such methods are described, for example, in Kenneth, A et al: Chemical Stability of Pharmaceuticals: A Handbook for Pharmacists, and in Peters et al., Pharmacokinetc analysis: A Practical Approach (1996). Reference is also made to “Pharmacokinetics”, M Gibaldi & D Perron, published by Marcel Dekker, 2^{nd} Rev. edition (1982), which describes pharmacokinetic parameters such as t alpha and t beta half-lives (t½ alpha, t½ beta) and area under curve (AUC).

Half-lives (t½ alpha and t½ beta) and AUC can be determined from a curve of serum concentration of conjugate or fusion against time. The WinNonlin analysis package (available from Pharsight Corp., Mountain View, Calif. 94040, USA) can be used, for example, to model the curve. In a first phase (the alpha phase) the drug composition (e.g., drug conjugate, noncovalent drug conjugate, drug fusion) is undergoing mainly distribution in the patient, with some elimination. A second phase (beta phase) is the terminal phase when the drug composition (e.g., drug conjugate, noncovalent drug conjugate, drug fusion) has been distributed and the serum concentration is decreasing as the drug composition is cleared from the patient. The t alpha half-life is the half-life of the first phase and the t beta half-life is the half-life of the second phase. Thus, the present invention provides a drug composition (e.g., drug conjugate, noncovalent drug conjugate, drug fusion) or a composition comprising a drug composition (e.g., drug conjugate, noncovalent drug conjugate, drug fusion) according to the invention having a toe half-life in the range of 15 minutes or more. In one embodiment, the lower end of the range is 30 minutes, 45 minutes, 1 hour, 2 hours, 3 hours, 4 hours, 5 hours, 6 hours, 7 hours, 8 hours, 9 hours, 10 hours, 11 hours or 12 hours. In addition, or alternatively, a drug composition (e.g., drug conjugate, noncovalent drug conjugate, drug fusion) or composition according to the invention will have a ta half-life in the range of up to and including 12 hours. In one embodiment, the upper end of the range is 11, 10, 9, 8, 7, 6 or 5 hours. An example of a suitable range is 1 to 6 hours, 2 to 5 hours or 3 to 4 hours.

Advantageously, the present invention provides drug compositions (e.g., drug conjugates, noncovalent drug conjugates, drug fusions) having a tβ half-life in the range of 2.5 hours or more. In one embodiment, the lower end of the range is 3 hours, 4 hours, 5 hours, 6 hours, 7 hours, 8 hours, 9 hours, 10 hours, 11 hours, or 12 hours. In some embodiments, the drug compositions (e.g., drug conjugates, noncovalent drug conjugates, drug fusions) have a tβ half-life in the range of up to and including 21 days. In one embodiment, the upper end of the range is 12 hours, 24 hours, 2 days, 3 days, 5 days, 10 days, 15 days or 20 days. In particular embodiments, a drug composition (e.g., drug conjugate, noncovalent drug conjugate, drug fusion) according to the invention will have a tβ half-life in the range 12 to 60 hours. In a further embodiment, it will be in the range 12 to 48 hours. In a further embodiment still, it will be in the range 12 to 26 hours.

In addition, or alternatively to the above criteria, the present invention provides drug compositions (e.g., drug conjugates, noncovalent drug conjugates, drug fusions) having an AUC value (area under the curve) in the range of 0.01 mg·min/mL or more, or 1 mg·min/mL or more. In one embodiment, the lower end of the range is 0.01, 0.1, 1, 5, 10, 15, 20, 30, 100, 200 or 300 mg·min/mL. In particular embodiments, the drug composition (e.g., drug conjugate, noncovalent drug conjugate, drug fusion) has an AUC in the range of up to 600 mg·min/mL. In one embodiment, the upper end of the range is 500, 400, 300, 200, 150, 100, 75 or 50 mg·min/mL. In other embodiments, the drug composition (e.g., drug conjugate, noncovalent drug conjugate, drug fusion) has an AUC in the range selected from the group consisting of the following: 15 to 150 mg·min/mL, 15 to 100 mg·min/mL, 15 to 75 mg·min/mL, 15 to 50 mg·min/mL, 0.01 to 50 mg·min/mL, 0.1 to 50 mg·min/mL, 1 to 50 mg·min/mL, 5 to 50 mg·min/mL, and 10 to 50 mg·min/mL.

The invention relates to drug compositions (e.g., drug conjugates, noncovalent drug conjugates, drug fusions) that comprise a drug and a polypeptide binding moiety that contains a binding site (e.g., an antigen-binding site) that has binding specificity for a polypeptide that enhances serum half-life in vivo. In preferred embodiments of drug compositions, the polypeptide binding moiety that contains a binding site (e.g., an antigen-binding site) that has binding specificity for a polypeptide that enhances serum half-life in vivo, has binding specificity for serum albumin.

In some embodiments, the drug composition comprises a drug that is covalently bonded to a polypeptide binding moiety that contains a binding site (e.g., an antigen-binding site) that has binding specificity for a polypeptide that enhances serum half-life in vivo. In these embodiments, the drug can be covalently bonded to the polypeptide binding domain at any suitable position, such as the amino-terminus, the carboxyl-terminus or through suitable amino acid side chains (e.g., the amino group of lysine).

In other embodiments, the drug composition comprises a drug that is noncovalently bonded to a polypeptide binding moiety that contains a binding site (e.g., an antigen-binding site) that has binding specificity for a polypeptide that enhances serum half-life in vivo. In such embodiments, the drug can be noncovalently bonded to the antigen-binding fragment directly (e.g., through electrostatic interaction, hydrophobic interaction) or indirectly (e.g., through noncovalent binding of complementary binding partners (e.g., biotin and avidin), wherein one partner is covalently bonded to drug and the complementary binding partner is covalently bonded to the antigen-binding fragment). When complementary binding partners are employed, one of the binding partners can be covalently bonded to the drug directly or through a suitable linker moiety, and the complementary binding partner can be covalently bonded to the polypeptide binding domain directly or through a suitable linker moiety.

In other embodiments, the drug composition is a fusion protein that comprises a polypeptide binding moiety that contains a binding site (e.g., an antigen-binding site) that has binding specificity for a polypeptide that enhances serum half-life in vivo and a polypeptide drug. The fusion proteins comprise a continuous polypeptide chain, said chain comprising a polypeptide binding moiety that contains a binding site (e.g., an antigen-binding site) that has binding specificity for a polypeptide that enhances serum half-life in vivo as a first moiety, and a polypeptide drug as a second moiety, which are present as discrete parts (moieties) of the polypeptide chain. The first and second moieties can be directly bonded to each other through a peptide bond, or linked through a suitable amino acid, or peptide or polypeptide linker. Additional moieties (e.g., third, fourth) and/or linker sequences can be present as appropriate. The first moiety can be in an N-terminal location, C-terminal location or internal relative to the second moiety (i.e., the polypeptide drug). In certain embodiments, the fusion protein comprises one or more one or more polypeptide binding moieties that contain a binding site that has binding specificity for a polypeptide that enhances serum half-life in vivo and one or more polypeptide drug moieties. In these embodiments, the fusion protein can comprise one to about ten (e.g., 1, 2, 3, 4, 5, 6, 7, 8, 9 or 10) polypeptide drug moieties that can be the same or different, and one to about twenty (e.g., 1, 2, 3, 4, 5, 6, 7, 8, 9, 10, 11, 12, 13, 14, 15, 16, 17, 18 19 or 20) polypeptide binding moieties that contain a binding site that has binding specificity for a polypeptide that enhances serum half-life in vivo that can be the same or different.

The polypeptide binding moieties that contain a binding site that has binding specificity for a polypeptide that enhances serum half-life in vivo and polypeptide drug moieties can be present in any desired location. For example, proceeding from the amino terminus to the carboxyl terminus, the moieties can be present in the following order: one or more polypeptide binding moieties, one or more polypeptide drug moieties, one or more polypeptide binding moieties. In another example, proceeding from the amino terminus to the carboxyl terminus, the moieties can be present in the following order: one or more polypeptide binding moieties, one or more polypeptide drug moieties, one or more polypeptide binding moieties, one or more polypeptide drug moieties, one or more polypeptide binding moieties. As described herein, the polypeptide binding moieties and polypeptide drug moieties can be directly bonded to each other through a peptide bond, or linked through a suitable amino acid, or peptide or polypeptide linker.

In certain embodiments, the fusion protein is a continuous polypeptide chain that has the formula (amino-terminal to carboxy-terminal):

a-(P)n2-b-(X)n1-c-(Q)n3-d or a-(Q)n3-b-(X)n1-c-(P)n2-d

wherein X is a polypeptide drug;

P and Q are each independently a polypeptide binding moiety that contains a binding site that has binding specificity for a polypeptide that enhances serum half-life in vivo;

a, b, c and d are each independently absent or one to about 100 amino acid residues;

n1, n2 and n3 represent the number of X, P or Q moieties present, respectively;

n1 is one to about 10;

n2 is zero to about 10; and

n3 is zero to about 10,

with the proviso that both n2 and n3 are not zero; and

with the proviso that when n1 and n2 are both one and n3 is zero, X does not comprise an antibody chain or a fragment of an antibody chain.

In some embodiments, n2 is one, two, three, four, five or six, and n3 is zero. In other embodiments, n3 is one, two, three, four, five or six, and n2 is zero. In other embodiments, n1, n2 and n3 are each one.

In certain embodiments, X does not comprises an antibody chain or a fragment of an antibody chain.

In preferred embodiments, P and Q are each independently a polypeptide binding moiety that has binding specificity for serum albumin.

In particularly preferred embodiments, the drug composition (e.g., drug conjugate, noncovalent drug conjugate, drug fusion) comprises a polypeptide binding moiety that contains a binding site (e.g., an antigen-binding site) that has binding specificity for a polypeptide that enhances serum half-life in vivo, wherein the polypeptide binding domain is an antigen-binding fragment of an antibody that has binding specificity for serum albumin.

The invention also relates to a method is for increasing the in vivo serum half-life of a drug, comprising bonding a drug to a polypeptide binding moiety having a binding site that has binding specificity for a polypeptide that enhances serum half-life in vivo, whereby a drug composition (e.g., drug conjugate, noncovalent drug conjugate, drug fusion) that has a longer in vivo serum half-life, relative to drug, is produced.

In some embodiments, the method is for increasing the in vivo serum half-life of a drug without substantially reducing the activity of the drug, comprising bonding a drug to a polypeptide binding moiety having a binding site that has binding specificity for a polypeptide that enhances serum half-life in vivo, whereby a drug composition (e.g., drug conjugate, noncovalent drug conjugate, drug fusion) that has a longer in vivo serum half-life relative to said drug, and has at least about 90% of the activity of said drug, is produced.

In other embodiments, the method is for increasing the in vivo serum half-life of a drug and reducing the immunogenicity of the drug, comprising bonding a drug to a polypeptide binding moiety having a binding site that has binding specificity for a polypeptide that enhances serum half-life in vivo, whereby a drug composition (e.g., drug conjugate, noncovalent drug conjugate, drug fusion) that has a longer in vivo serum half-life relative to drug, and is less immunogenic than said drug, is produced.

In other embodiments, the method is for decreasing the immunogenicity of a drug without substantially reducing the activity of the drug, comprising bonding a drug to a polypeptide binding moiety having a binding site that has binding specificity for a polypeptide that enhances serum half-life in vivo, whereby a drug composition (e.g., drug conjugate, noncovalent drug conjugate, drug fusion) that is less immunogenic than said drug, and has at least about 90% of the activity of said drug is produced.

In other embodiments, the method is for increasing the in vivo serum half-life of a drug, and reducing the immunogenicity of the drug without substantially reducing the activity of the drug, comprising bonding a drug to a polypeptide binding moiety having a binding site that has binding specificity for a polypeptide that enhances serum half-life in vivo, whereby a drug composition (e.g., drug conjugate, noncovalent drug conjugate, drug fusion) that has a longer in vivo serum half-life relative to said drug, is less immunogenic than said drug, and has at least about 90% of the activity of said drug is produced.

The drug and the polypeptide binding moiety having a binding site that has binding specificity for a polypeptide that enhances serum half-life in vivo can be bonded via a covalent bond (e.g., peptide bond) or noncovalent bond, with or without the use of linkers, as described herein. In some embodiments, the drug and the polypeptide binding moiety having a binding site that has binding specificity for a polypeptide that enhances serum half-life in vivo are bonded via a covalent bond. For example, the drug composition produced is a drug conjugate or drug fusion. In other embodiments, the drug and the polypeptide binding moiety having a binding site that has binding specificity for a polypeptide that enhances serum half-life in vivo are bonded via a noncovalent bond, and the drug composition is a noncovalent drug conjugate.

The drug composition produced using the method can have greater activity (e.g., in vivo activity) than the drug. In some embodiments, the method is for producing a drug composition that has greater activity (e.g., in vivo activity) than drug alone, comprising bonding a drug to a polypeptide binding moiety having a binding site that has binding specificity for a polypeptide that enhances serum half-life in vivo, whereby a drug composition (e.g., drug conjugate, noncovalent drug conjugate, drug fusion) that has greater activity, relative to drug, is produced. In such embodiments, preferably, the activity of the drug composition is greater than the activity of the drug as described herein.

In preferred embodiments, the polypeptide binding moiety has binding specificity for serum albumin. In particularly preferred embodiments, the polypeptide binding moiety is an antigen-binding fragment of an antibody that has binding specificity for serum albumin.

In certain embodiments, the method comprises selecting said polypeptide binding moiety from one or more polypeptides (e.g., antigen-binding fragments of an antibody that has binding specificity for serum albumin), wherein the selected polypeptide binding moiety binds a polypeptide that enhances serum half-life in vivo with a KD of at least about 5 mM.

The invention also relates to use of a polypeptide binding moiety having a binding site that has binding specificity for a polypeptide that enhances serum half-life in vivo for the manufacture of medicament, the medicament comprising a drug composition (e.g., drug conjugate, noncovalent drug conjugate, drug fusion) in which a drug is bonded to said polypeptide binding moiety, for increasing in vivo serum half-life of the drug.

In some embodiments, the use is for the manufacture of a medicament, the medicament comprising a drug composition (e.g., drug conjugate, noncovalent drug conjugate, drug fusion) in which a drug is bonded to said polypeptide binding moiety, for increasing in vivo serum half-life of the drug without reducing the activity of the drug by more than about 10%.

In other embodiments, the use is for the manufacture of a medicament, the medicament comprising a drug composition (e.g., drug conjugate, noncovalent drug conjugate, drug fusion) in which a drug is bonded to said polypeptide binding moiety, for increasing in vivo serum half-life of the drug and reducing the immunogenicity of the drug.

In other embodiments, the use is for the manufacture of a medicament, the medicament comprising a drug composition (e.g., drug conjugate, noncovalent drug conjugate, drug fusion) in which a drug is bonded to said polypeptide binding moiety, for decreasing the immunogenicity of a drug without reducing the activity of the drug by more than about 10%.

In other embodiments, the use is for the manufacture of a medicament, the medicament comprising a drug composition (e.g., drug conjugate, noncovalent drug conjugate, drug fusion) in which a drug is bonded to said polypeptide binding moiety, for increasing in vivo serum half-life of the drug, and reducing the immunogenicity of the drug without reducing the activity of the drug by more than about 10%.

The drug composition can comprise a drug and polypeptide binding moiety having a binding site that has binding specificity for a polypeptide that enhances serum half-life in vivo that are bonded via a covalent bond (e.g., peptide bond) or noncovalent bond, with or without the use of linkers, as described herein. In some embodiments, the drug and the polypeptide binding moiety having a binding site that has binding specificity for a polypeptide that enhances serum half-life in vivo are bonded via a covalent bond. For example, the drug composition can be a drug conjugate or drug fusion. In other embodiments, the drug and the polypeptide binding moiety having a binding site that has binding specificity for a polypeptide that enhances serum half-life in vivo are bonded via a noncovalent bond, and the drug composition is a noncovalent drug conjugate.

In certain embodiments, the use is for the manufacture of a medicament, the medicament comprising a drug composition (e.g., drug conjugate, noncovalent drug conjugate, drug fusion) in which a drug is bonded to said polypeptide binding moiety, for increasing the activity (e.g., in vivo activity) than said drug. In such embodiments, preferably, the activity of the drug composition is greater than the activity of the drug as described herein.

In preferred embodiments, the polypeptide binding moiety has binding specificity for serum albumin. In particularly preferred embodiments, the polypeptide binding moiety is an antigen-binding fragment of an antibody that has binding specificity for serum albumin.

Antigen-Binding Fragment of an Antibody that Binds Serum Albumin

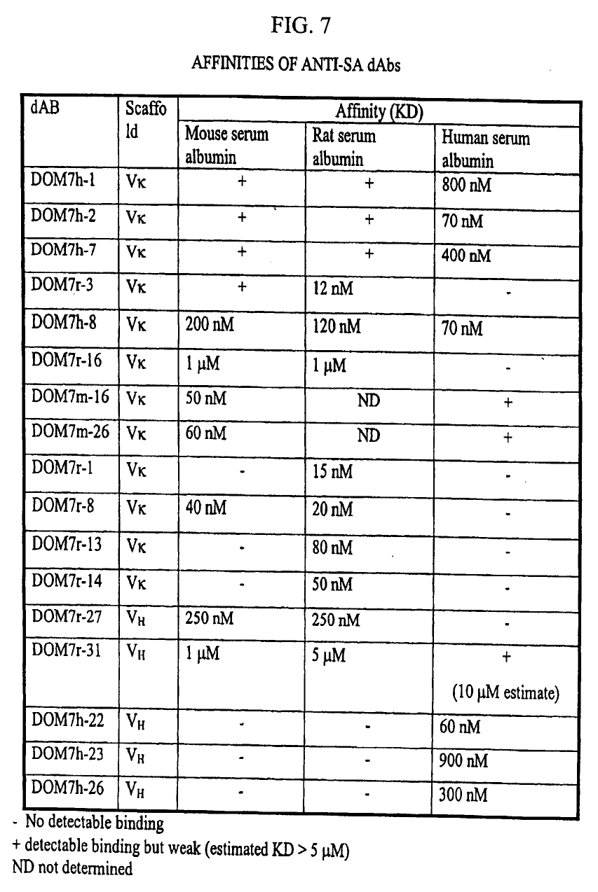

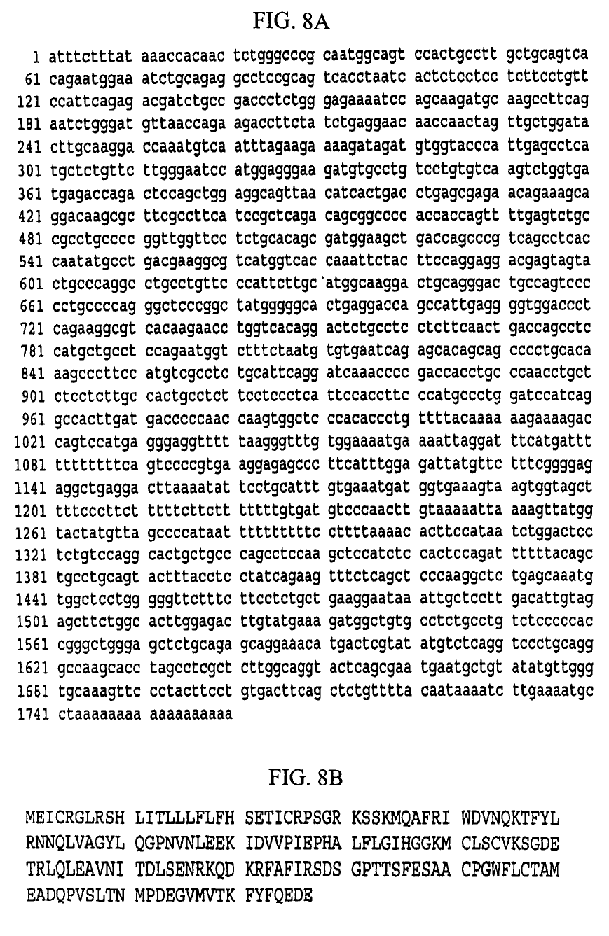

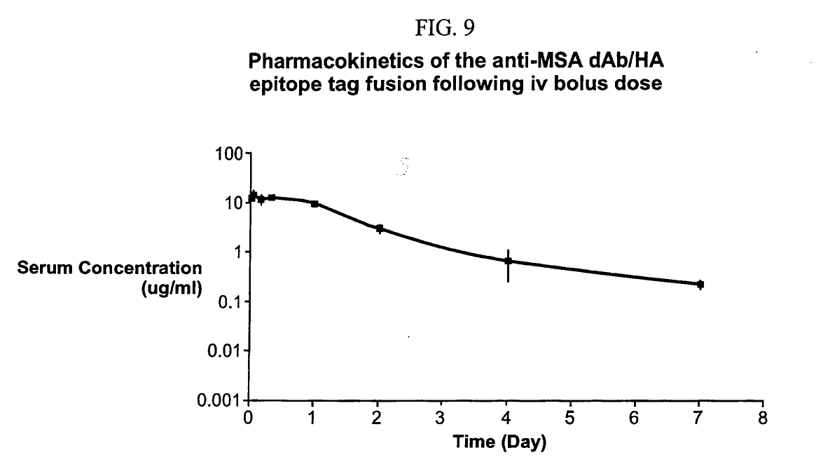

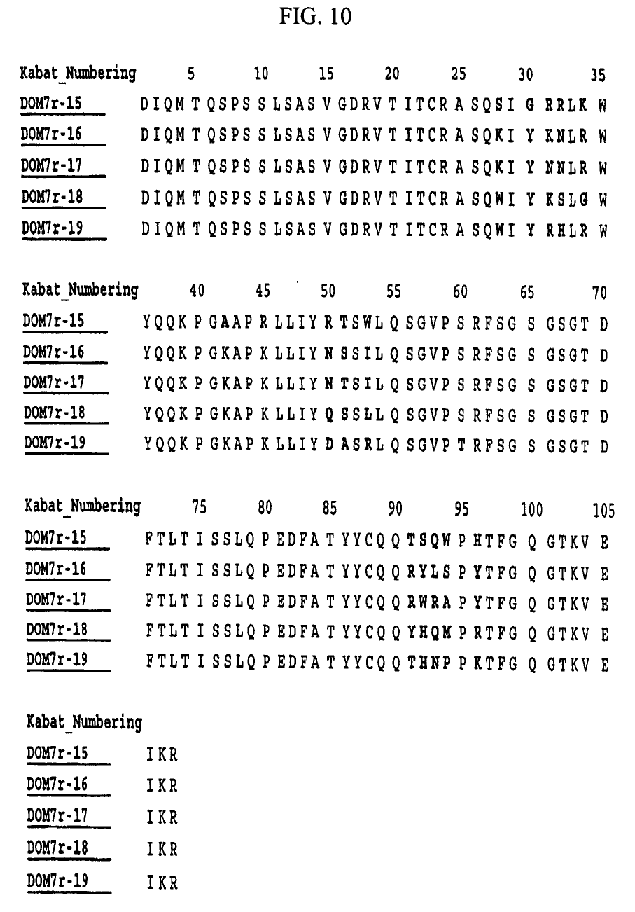

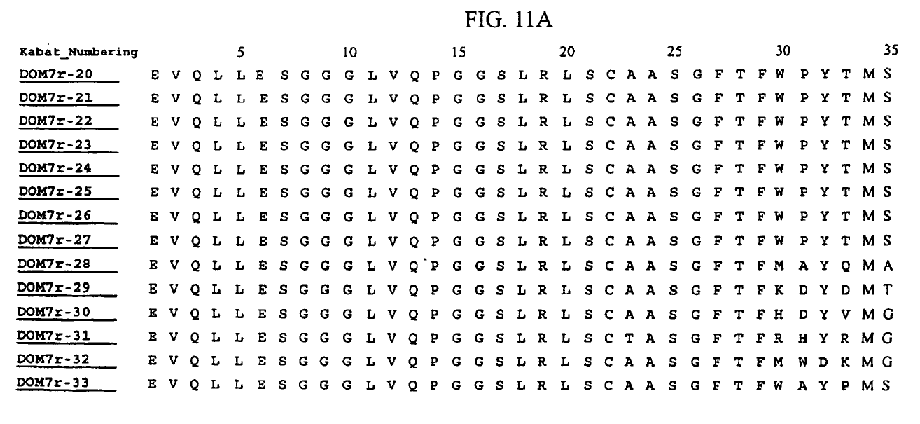

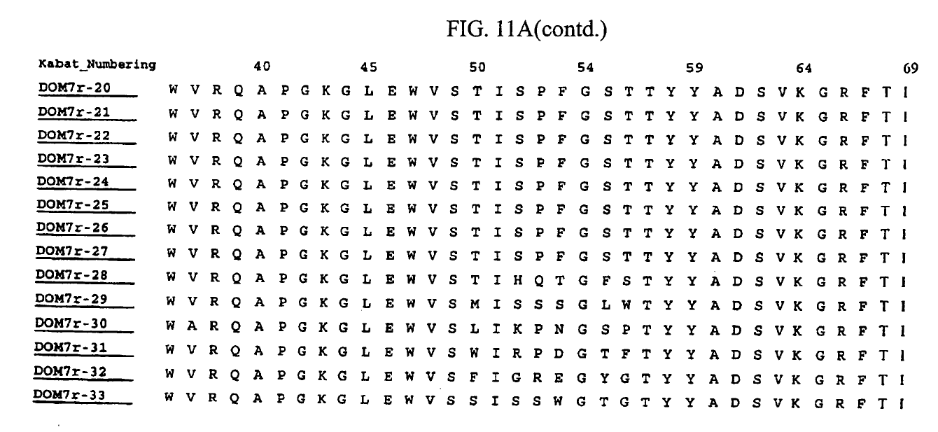

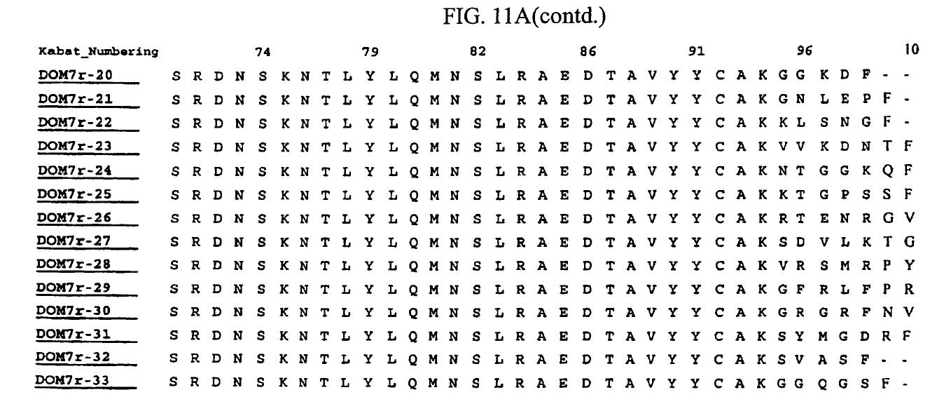

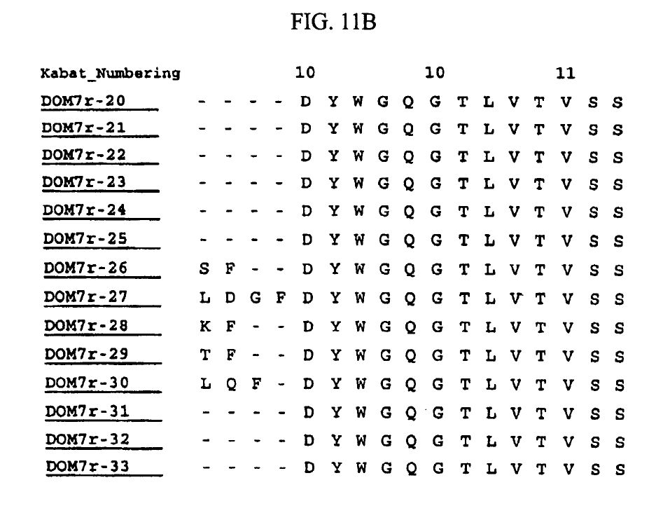

The drug conjugates, noncovalent drug conjugates and drug fusions of the invention comprise an (i.e., one or more) antigen-binding fragment of an antibody that binds serum albumin. The antigen-binding fragment can have binding specificity for serum albumin of an animal to which the drug conjugate or drug fusion will be administered. Preferably, the antigen-binding fragment has binding specificity for human serum albumin. However, veterinary applications are contemplated and the antigen-binding fragment can have binding specificity for serum albumin from a desired animal, for example serum albumin from dog, cat, horse, cow, chicken, sheep, pig, goat, deer, mink, and the like. In some embodiments the antigen-binding fragment has binding specificity for serum albumin from more than one species. For example, as described herein, human dAbs that have binding specificity for rat serum albumin and mouse serum albumin, and a dAb that has binding specificity for rat, mouse and human serum albumin have been produced. (Table 1 and FIG. 7) Such dAbs provide the advantage of allowing preclinical and clinical studies using the same drug conjugate or drug fusion and obviate the need to conduct preclinical studies with a suitable surrogate drug fusion or drug conjugate.

Antigen-binding fragments suitable for use in the invention include, for example, Fab fragments, Fab′ fragments, F(ab′)₂ fragments, Fv fragments (including single chain Fv (scFv) and disulfide bonded Fv), a single variable domain, and dAbs (V_{H}, V_{L}). Such antigen-binding fragments can be produced using any suitable method, such as by proteolysis of an antibody using pepsin, papain or other protease having the requisite cleavage specificity, or using recombinant techniques. For example, Fv fragments can be prepared by digesting an antibody with a suitable protease or using recombinant DNA technology. For example, a nucleic acid can be prepared that encodes a light chain variable region and heavy chain variable region that are connected by a suitable peptide linker, such as a chain of two to about twenty Glycyl residues. The nucleic acid can be introduced into a suitable host (e.g., E. coli) using any suitable technique (e.g., transfection, transformation, infection), and the host can be maintained under conditions suitable for expression of a single chain Fv fragment. A variety of antigen-binding fragments of antibodies can be prepared using antibody genes in which one or more stop codons have been introduced upstream of the natural stop site. For example, an expression construct encoding a F(ab′)₂ portion of an immunoglobulin heavy chain can be designed by introducing a translation stop codon at the 3′ end of the sequence encoding the hinge region of the heavy chain. The drug conjugates, noncovalent drug conjugates and drug fusions of the invention can comprise the individual heavy and light chains of antibodies that bind serum albumin or portions of the individual chains that bind serum albumin (e.g., a single V_{H}, V_{κ} or V_{λ}).

Antibodies and antigen-binding fragments thereof which bind a desired serum albumin (e.g., human serum albumin) can be selected from a suitable collection of natural or artificial antibodies or raised against an appropriate immunogen in a suitable host. For example, antibodies can be raised by immunizing a suitable host (e.g., mouse, human antibody-transgenic mouse, rat, rabbit, chicken, goat, non-human primate (e.g., monkey)) with serum albumin (e.g., isolated or purified human serum albumin) or a peptide of serum albumin (e.g., a peptide comprising at least about 8, 9, 10, 11, 12, 15, 20, 25, 30, 33, 35, 37, or 40 amino acid residues). Antibodies and antigen-binding fragments that bind serum albumin can also be selected from a library of recombinant antibodies or antigen-binding fragments, such as a phage display library. Such libraries can contain antibodies or antigen-binding fragments of antibodies that contain natural or artificial amino acid sequences. For example, the library can contain Fab fragments which contain artificial CDRs (e.g., random amino acid sequences) and human framework regions. (See, for example, U.S. Pat. No. 6,300,064 (Knappik, et al).) In other examples, the library contains scFv fragments or dAbs (single V_{H}, single V_{κ} or single V_{λ}) with sequence diversity in one or more CDRs, (See, e.g., WO 99/20749 (Tomlinson and Winter), WO 03/002609 A2 (Winter et al), WO 2004/003019A2 (Winter et al.).)

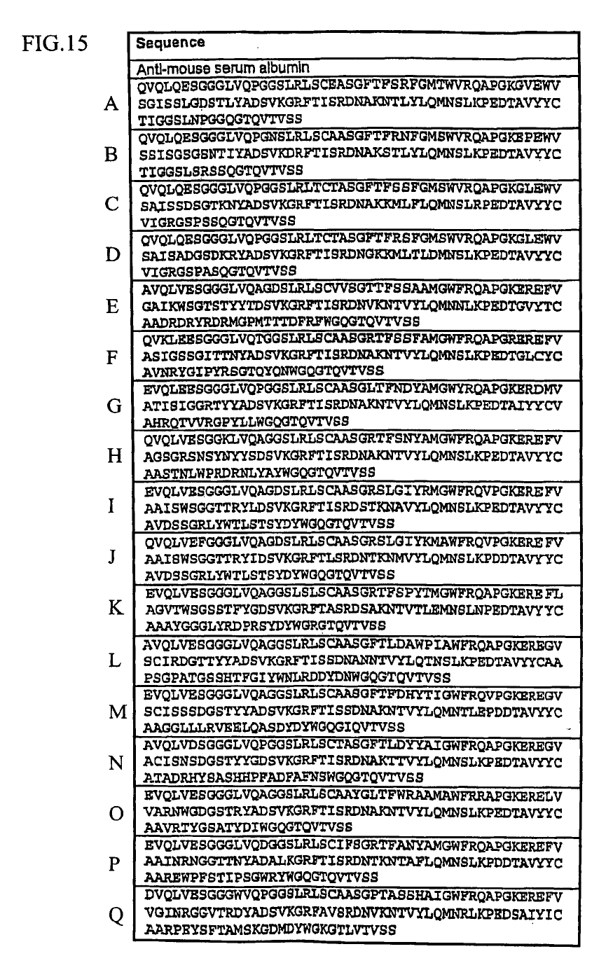

Suitable antibodies and antigen-binding fragments thereof that bind serum albumin include, for example, human antibodies and antigen-binding fragments thereof, humanized antibodies and antigen-binding fragments thereof, chimeric antibodies and antigen-binding fragments thereof, rodent (e.g., mouse, rat) antibodies and antigen-binding fragments thereof, and Camelid antibodies and antigen-binding fragments thereof. In certain embodiments, the drug conjugates, noncovalent drug conjugates and drug fusions comprises a Camelid V_{HH} that binds serum albumin, Camelid V_{HH}_{^{S}} are immunoglobulin single variable domain polypeptides which are derived from heavy chain antibodies that are naturally devoid of light chains. Such antibodies occur in Camelid species including camel, llama, alpaca, dromedary, and guanaco. V_{HH} molecules are about ten times smaller than IgG molecules, and as single polypeptides, are very stable and resistant to extreme pH and temperature conditions. Suitable Camelid V_{HH} that bind serum albumin include those disclosed in WO 2004/041862 (Ablynx N. V.) and herein (FIG. 15 and SEQ ID NOS:73-84). In certain embodiments, the Camelid V_{HH} binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with SEQ ID NO: 68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, or SEQ ID NO:84. Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

Preparation of the Immunizing Antigen, and Polyclonal and Monoclonal antibody production can be performed using any suitable technique. A variety of methods have been described. (See, e.g., Kohler et al., Nature, 256:495-497 (1975) and Eur. J. Immunol. 6:511-519 (1976); Milstein et al., Nature 266:550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991).) Generally, where a monoclonal antibody is desired, a hybridoma is produced by fusing suitable cells from an immortal cell line (e.g., a myeloma cell line such as SP2/0, P3X63Ag8.653 or a heteromyeloma) with antibody-producing cells. Antibody-producing cells can be obtained from the peripheral blood or, preferably the spleen or lymph nodes, of humans, human-antibody transgenic animals or other suitable animals immunized with the antigen of interest. Cells that produce antibodies of human origin (e.g., a human antibody) can be produced using suitable methods, for example, fusion of a human antibody-producing cell and a heteromyeloma or trioma, or immortalization of an activated human B cell via infection with Epstein Barr virus. (See, e.g., U.S. Pat. No. 6,197,582 (Trakht); Niedbala et al., Hybridoma, 17:299-304 (1998); Zanella et al., J Immunol Methods, 156:205-215 (1992); Gustafsson et al., Hum Antibodies Hybridomas, 2:26-32 (1991).) The fused or immortalized antibody-producing cells (hybridomas) can be isolated using selective culture conditions, and cloned by limiting dilution. Cells which produce antibodies with the desired specificity can be identified using a suitable assay (e.g., ELISA).

Antibodies also can be prepared directly (e.g., synthesized or cloned) from an isolated antigen-specific antibody producing cell (e.g., a cell from the peripheral blood or, preferably the spleen or lymph nodes determined to produce an antibody with desired specificity), of humans, human-antibody transgenic animals or other suitable animals immunized with the antigen of interest (see, e.g., U.S. Pat. No. 5,627,052 (Schrader)).

When the drug conjugate, noncovalent drug conjugate or drug fusion is for administration to a human, the antibody or antigen-binding fragment thereof that binds serum albumin (e.g., human serum albumin) can be a human, humanized or chimeric antibody or an antigen-binding fragment of such an antibody. These types of antibodies and antigen-binding fragments are less immunogenic or non-immunogenic in humans and provide well-known advantages. For example, drug conjugates, noncovalent drug conjugates or drug fusions that contain an antigen-binding fragment of a human, humanized or chimeric antibody can be administered repeatedly to a human with less or no loss of efficacy (compared with other fully immunogenic antibodies) due to elaboration of human antibodies that bind to the drug conjugate or drug fusion. When the drug conjugate, noncovalent drug conjugate or drug fusion is intended for veterinary administration, analogous antibodies or antigen-binding fragments can be used. For example, CDRs from a murine or human antibody can be grafted onto framework regions from a desired animal, such as a horse or cow.

Human antibodies and nucleic acids encoding same can be obtained, for example, from a human or from human-antibody transgenic animals. Human-antibody transgenic animals (e.g., mice) are animals that are capable of producing a repertoire of human antibodies, such as XENOMOUSE (Abgenix, Fremont, Calif.), HUMAB-MOUSE, KIRIN TC MOUSE or KM-MOUSE (MEDAREX, Princeton, N.J.). Generally, the genome of human-antibody transgenic animals has been altered to include a transgene comprising DNA from a human immunoglobulin locus that can undergo functional rearrangement. An endogenous immunoglobulin locus in a human-antibody transgenic animal can be disrupted or deleted to eliminate the capacity of the animal to produce antibodies encoded by an endogenous gene. Suitable methods for producing human-antibody transgenic animals are well known in the art. (See, for example, U.S. Pat. Nos. 5,939,598 and 6,075,181 (Kucherlapati et al), U.S. Pat. Nos. 5,569,825, 5,545,806, 5,625,126, 5,633,425, 5,661,016, and 5,789,650 (Lonberg et al), Jakobovits et al., Proc. Natl. Acad. Sci. USA, 90:2551-2555 (1993), Jakobovits et al., Nature, 362:255-258 (1993), Jakobovits et al. WO 98/50433, Jakobovits et al. WO 98/24893, Lonberg et al. WO 98/24884, Lonberg et al. WO 97/13852, Lonberg et al. WO 94/25585, Lonberg et al. EP 0 814 259 A2, Lonberg et al. GB 2 272 440 A, Lonberg et al., Nature 368:856-859 (1994), Lonberg et al., Int Rev Immunol 13(1):65-93 (1995), Kucherlapati et al. WO 96/34096, Kucherlapati et al. EP 0 463 151 B1, Kucherlapati et al. EP 0 710 719 A1, Surani et al. U.S. Pat. No. 5,545,807, Bruggemann et al. WO 90/04036, Bruggemann et al. EP 0 438 474 B1, Taylor et al., Int. Immunol. 6(4)579-591 (1994), Taylor et al., Nucleic Acids Research 20(23):6287-6295 (1992), Green et al., Nature Genetics 7:13-21 (1994), Mendez et al., Nature Genetics 15:146-156 (1997), Tuaillon et al., Proc Natl Acad Sci USA 90(8):3720-3724 (1993) and Fishwild et al., Nat Biotechnol 14(7):845-851 (1996), the teachings of each of the foregoing are incorporated herein by reference in their entirety.)

Human-antibody transgenic animals can be immunized with a suitable antigen (e.g., human serum albumin), and antibody producing cells can be isolated and fused to form hybridomas using conventional methods. Hybridomas that produce human antibodies having the desired characteristics (e.g., specificity, affinity) can be identified using any suitable assay (e.g., ELISA) and, if desired, selected and subcloned using suitable culture techniques.

Humanized antibodies and other CDR-grafted antibodies can be prepared using any suitable method. The CDRs of a CDR-grafted antibody can be derived from a suitable antibody which binds a serum albumin (referred to as a donor antibody). Other sources of suitable CDRs include natural and artificial serum albumin-specific antibodies obtained from human or nonhuman sources, such as rodent (e.g., mouse, rat, rabbit), chicken, pig, goat, non-human primate (e.g., monkey) or a library.

The framework regions of a humanized antibody are preferably of human origin, and can be derived from any human antibody variable region having sequence similarity to the analogous or equivalent region (e.g., heavy chain variable region or light chain variable region) of the antigen-binding region of the donor antibody. Other sources of framework regions of human origin include human variable region consensus sequences. (See, e.g., Kettleborough, C. A. et al., Protein Engineering 4:773-783 (1991); Carter et al., WO 94/04679; Kabat, E. A., et al., Sequences of Proteins of Immunological Interest, Fifth Edition, U.S. Department of Health and Human Services, U.S. Government Printing Office (1991)). Other types of CDR grafted antibodies can contain framework regions of suitable origin, such as framework regions encoded by germline antibody gene segments from horse, cow, dog, cat and the like.

Framework regions of human origin can include amino acid substitutions or replacements, such as “back mutations” which replace an amino acid residue in the framework region of human or animal origin with a residue from the corresponding position of the donor antibody. One or more mutations in the framework region can be made, including deletions, insertions and substitutions of one or more amino acids. Variants can be produced by a variety of suitable methods, including mutagenesis of nonhuman donor or acceptor human chains. (See, e.g., U.S. Pat. Nos. 5,693,762 (Queen et al.) and 5,859,205 (Adair et al), the entire teachings of which are incorporated herein by reference.)

Constant regions of antibodies, antibody chains (e.g., heavy chain, light chain) or fragments or portions thereof, if present, can be derived from any suitable source. For example, constant regions of human, humanized and certain chimeric antibodies, antibody chains (e.g., heavy chain, light chain) or fragments or portions thereof, if present can be of human origin and can be derived from any suitable human antibody or antibody chain. For example, a constant region of human origin or portion thereof can be derived from a human k or X light chain, and/or a human γ (e.g., γ1, γ2, γ3, γ4), μ, α (e.g., α1, α2), δ or ε heavy chain, including allelic variants. In certain embodiments, the antibody or antigen-binding fragment (e.g., antibody of human origin, human antibody) can include amino acid substitutions or replacements that alter or tailor function (e.g., effector function). For example, a constant region of human origin (e.g., γ1 constant region, γ2 constant region) can be designed to reduce complement activation and/or Fc receptor binding. (See, for example, U.S. Pat. Nos. 5,648,260 (Winter et al), 5,624,821 (Winter et al.) and 5,834,597 (Tso et al), the entire teachings of which are incorporated herein by reference.) Preferably, the amino acid sequence of a constant region of human origin that contains such amino acid substitutions or replacements is at least about 95% identical over the full length to the amino acid sequence of the unaltered constant region of human origin, more preferably at least about 99% identical over the full length to the amino acid sequence of the unaltered constant region of human origin.

Humanized antibodies, CDR grafted antibodies or antigen-binding fragments of a humanized or CDR grafted antibody can be prepared using any suitable method. Several such methods are well-known in the art. (See, e.g., U.S. Pat. No. 5,225,539 (Winter), U.S. Pat. No. 5,530,101 (Queen et al.).) The portions of a humanized or CDR grafted antibody (e.g., CDRs, framework, constant region) can be obtained or derived directly from suitable antibodies (e.g., by de novo synthesis of a portion), or nucleic acids encoding an antibody or chain thereof having the desired property (e.g., binds serum albumin) can be produced and expressed. To prepare a portion of a chain, one or more stop codons can be introduced at the desired position. For example, nucleic acid (e.g., DNA) sequences coding for humanized or CDR grafted variable regions can be constructed using PCR mutagenesis methods to alter existing DNA sequences. (See, e.g., Kamman, M., et al., Nucl. Acids Res. 17:5404 (1989).) PCR primers coding for the new CDRs can be hybridized to a DNA template of a previously humanized variable region which is based on the same, or a very similar, human variable region (Sato, K., et al., Cancer Research 53:851-856 (1993)). If a similar DNA sequence is not available for use as a template, a nucleic acid comprising a sequence encoding a variable region sequence can be constructed from synthetic oligonucleotides (see e.g., Kolbinger, F., Protein Engineering 8:971-980 (1993)). A sequence encoding a signal peptide can also be incorporated into the nucleic acid (e.g., on synthesis, upon insertion into a vector). The natural signal peptide sequence from the acceptor antibody, a signal peptide sequence from another antibody or other suitable sequence can be used (see, e.g., Kettleborough, C. A., Protein Engineering 4:112-783 (1991)). Using these methods or other suitable methods, variants can be readily produced. In one embodiment, cloned variable regions can be mutated, and sequences encoding variants with the desired specificity can be selected (e.g., from a phage library; see, e.g., U.S. Pat. No. 5,514,548 (Krebber et al.) and WO 93/06213 (Hoogenboom et al.)).

The antibody or antigen-binding fragment that binds serum albumin can be a chimeric antibody or an antigen-binding fragment of a chimeric antibody. The chimeric antibody or antigen-binding fragment thereof comprises a variable region from one species (e.g., mouse) and at least a portion of a constant region from another species (e.g., human). Chimeric antibodies and antigen-binding fragments of chimeric antibodies can be prepared using any suitable method. Several suitable methods are well-known in the art. (See, e.g., U.S. Pat. No. 4,816,567 (Cabilly et al), U.S. Pat. No. 5,116,946 (Capon et al.).)

A preferred method for obtaining antigen-binding fragments of antibodies that bind serum albumin comprises selecting an antigen-binding fragment (e.g., scFvs, dAbs) that has binding specificity for a desired serum albumin from a repertoire of antigen-binding fragments. For example, as described herein dAbs that bind serum albumin can be selected from a suitable phage display library. A number of suitable bacteriophage display libraries and selection methods (e.g., monovalent display and multivalent display systems) have been described. (See, e.g., Griffiths et al., U.S. Pat. No. 6,555,313 B1 (incorporated herein by reference); Johnson et al., U.S. Pat. No. 5,733,743 (incorporated herein by reference); McCafferty et al., U.S. Pat. No. 5,969,108 (incorporated herein by reference); Mulligan-Kehoe, U.S. Pat. No. 5,702,892 (incorporated herein by reference); Winter, G. et al., Annu. Rev. Immunol. 72:433-455 (1994); Soumillion, P. et al., Appl. Biochem. Biotechnol. 47(2-3):175-189 (1994); Castagnoli, L. et al., Comb. Chem. High Throughput Screen, 4(2): 121-133 (2001); WO 99/20749 (Tomlinson and Winter); WO 03/002609 A2 (Winter et al); WO 2004/003019A2 (Winter et al.).) The polypeptides displayed in a bacteriophage library can be displayed on any suitable bacteriophage, such as a filamentous phage (e.g., fd, M13, F1), a lytic phage (e.g., T4, T7, lambda), or an RNA phage (e.g., MS2), for example, and selected for binding to serum albumin (e.g., human serum albumin).

Generally, a library of phage that displays a repertoire of polypeptides as fusion proteins with a suitable phage coat protein is used. Such a library can be produced using any suitable methods, such as introducing a library of phage vectors or phagemid vectors encoding the displayed antibodies or antigen-binding fragments thereof into suitable host bacteria, and culturing the resulting bacteria to produce phage (e.g., using a suitable helper phage or complementing plasmid if desired). The library of phage can be recovered from such a culture using any suitable method, such as precipitation and centrifugation.

The library can comprise a repertoire of antibodies or antigen-binding fragments thereof that contains any desired amount of amino acid sequence diversity. For example, the repertoire can contain antibodies or antigen-binding fragments thereof that have amino acid sequences that correspond to naturally occurring antibodies from a desired organism, and/or can contain one or more regions of random or randomized amino acid sequences (e.g., CDR sequences). The antibodies or antigen-binding fragments thereof in such a repertoire or library can comprise defined regions of random or randomized amino acid sequence and regions of common amino acid sequence. In certain embodiments, all or substantially all polypeptides in a repertoire are a desired type of antigen-binding fragment of an antibody (e.g., human V_{H} or human V_{L}). For example, each polypeptide in the repertoire can contain a V_{H}, a V_{L} or an Fv (e.g., a single chain Fv).

Amino acid sequence diversity can be introduced into any desired region of antibodies or antigen-binding fragments thereof using any suitable method. For example, amino acid sequence diversity can be introduced into a target region, such as a complementarity determining region of an antibody variable domain, by preparing a library of nucleic acids that encode the diversified antibodies or antigen-binding fragments thereof using any suitable mutagenesis methods (e.g., low fidelity PCR, oligonucleotide-mediated or site directed mutagenesis, diversification using NNK codons) or any other suitable method. If desired, a region of the antibodies or antigen-binding fragments thereof to be diversified can be randomized.

A suitable phage display library can be used to selected antibodies or antigen-binding fragments of antibodies that bind serum albumin and have other beneficial properties. For example, antibodies or antigen-binding fragments that resist aggregation when unfolded can be selected. Aggregation is influenced by polypeptide concentration and is thought to arise in many cases from partially folded or unfolded intermediates. Factors and conditions that favor partially folded intermediates, such as elevated temperature and high polypeptide concentration, promote irreversible aggregation. (Fink, A. L., Folding & Design 3:R1-R23 (1998).)

For example, storing purified polypeptides in concentrated form, such as a lyophilized preparation, frequently results in irreversible aggregation of at least a portion of the polypeptides. Also, production of a polypeptide by expression in biological systems, such as E. coli, often results in the formation of inclusion bodies which contain aggregated polypeptides. Recovering active polypeptides from inclusion bodies can be very difficult and require adding additional steps, such as a refolding step, to a biological production system.

Antibodies and antigen-binding fragments that resist aggregation and unfold reversibly when heated can be selected from a suitable phage display library. Generally, a phage display library comprising a repertoire of displayed antibodies or antigen-binding fragments thereof is heated to a temperature (Ts) at which at least a portion of the displayed antibodies or antigen-binding fragments thereof are unfolded, then cooled to a temperature (Tc) wherein Ts>Tc, whereby at least a portion of the antibodies or antigen-binding fragments thereof have refolded and a portion of the polypeptides have aggregated. Then, antibodies or antigen-binding fragments thereof that unfold reversibly and bind serum albumin are recovered at a temperature (Tr). The recovered antibody or antigen-binding fragment thereof that unfolds reversibly has a melting temperature (Tm), and preferably, the repertoire was heated to Ts, cooled to Tc and the antibody or antigen-binding fragment thereof that unfolds reversibly was isolated at Tr, such that Ts>Tm>Tc, and Ts>Tm>Tr. Generally, the phage display library is heated to about 80° C. and cooled to about room temperature or about 4° C. before selection. Antibodies or antigen-binding fragment thereof that unfold reversibly and resist aggregation can also be designed or engineered by replacing certain amino acid residue with residues that confer the ability to unfold reversibly. (See, WO 2004/101790 (Jespers et al), and U.S. Provisional Patent Application Nos. 60/470,340 (filed on May 14, 2003) and 60/554,021 (filed on Mar. 17, 2004) for detailed discussion of methods for selecting and for designing or engineering antibodies or antigen-binding fragments thereof that unfold reversibly. The teachings of WO 2004/101790 and both of the foregoing U.S. Provisional Patent Applications are incorporated herein by reference.).

Antibodies or antigen-binding fragments thereof that unfold reversibly and resist aggregation provide several advantages. For example, due to their resistance to aggregation, antibodies or antigen-binding fragments thereof that unfold reversibly can readily be produced in high yield as soluble proteins by expression using a suitable biological production system, such as E. coli. In addition, antibodies or antigen-binding fragments thereof that unfold reversibly can be formulated and/or stored at higher concentrations than conventional polypeptides, and with less aggregation and loss of activity. DOM7h-26 (SEQ ID NO:20) is a human V_{H} that unfolds reversibly.

Preferably, the antibody or antigen-binding fragment thereof that binds serum albumin comprises a variable domain (V_{H}, V_{κ}, V_{λ}) in which one or more of the framework regions (FR) comprise (a) the amino acid sequence of a human framework region, (b) at least 8 contiguous amino acids of the amino acid sequence of a human framework region, or (c) an amino acid sequence encoded by a human germline antibody gene segment, wherein said framework regions are as defined by Kabat. In certain embodiments, the amino acid sequence of one or more of the framework regions is the same as the amino acid sequence of a corresponding framework region encoded by a human germline antibody gene segment, or the amino acid sequences of one or more of said framework regions collectively comprise up to 5 amino acid differences relative to the amino acid sequence of said corresponding framework region encoded by a human germline antibody gene segment.

In other embodiments, the amino acid sequences of FR1, FR2, FR3 and FR4 are the same as the amino acid sequences of corresponding framework regions encoded by a human germline antibody gene segment, or the amino acid sequences of FR1, FR2, FR3 and FR4 collectively contain up to 10 amino acid differences relative to the amino acid sequences of corresponding framework regions encoded by said human germline antibody gene segments. In other embodiments, the amino acid sequence of said FR1, FR2 and FR3 are the same as the amino acid sequences of corresponding framework regions encoded by said human germline antibody gene segment.

In particular embodiments, the antigen binding fragment of an antibody that binds serum albumin comprises an immunoglobulin variable domain (e.g., V_{H}, V_{L}) based on a human germline sequence, and if desired can have one or more diversified regions, such as the complementarity determining regions. Suitable human germline sequence for V_{H} include, for example, sequences encoded by the V_{H} gene segments DP4, DP7, DP8, DP9, DP10, DP31, DP33, DP45, DP46, DP47, DP49, DP50, DP51, DP53, DP54, DP65, DP66, DP67, DP68 and DP69, and the JH segments JH1, JH2, JH3, JH4, JH4b, JH5 and JH6. Suitable human germline sequence for V_{L} include, for example, sequences encoded by the V_{κ} gene segments DPK1, DPK2, DPK3, DPK4, DPK5, DPK6, DPK7, DPK8, DPK9, DPK10, DPK12, DPK13, DPK15, DPK16, DPK18, DPK19, DPK20, DPK21, DPK22, DPK23, DPK24, DPK25, DPK26 and DPK28, and the Jκ segments Jκ 1, Jκ 2, Jκ 3, Jκ 4 and Jκ5.

In certain embodiments, the drug conjugate, noncovalent drug conjugate or drug fusion does not contain a mouse, rat and/or rabbit antibody that binds serum albumin or antigen-binding fragment of such an antibody.

The antigen-binding fragment can bind serum albumin with any desired affinity, on rate and off rate. The affinity (KD), on rate (Kₒₙ or kₐ) and off rate (K_{off} or k_{d}) can be selected to obtain a desired serum half-life for a particular drug. For example, it may be desirable to obtain a maximal serum half-life for a drug that neutralizes an inflammatory mediator of a chronic inflammatory disorder (e.g., a dAb that binds and neutralizes an inflammatory cytokine), while a shorter half-life may be desirable for a drug that has some toxicity (e.g., a chemorherapeutic agent). Generally, a fast on rate and a fast or moderate off rate for binding to serum albumin is preferred. Drug conjugates and drug fusions that comprise an antigen-binding fragment with these characteristics will quickly bind serum albumin after being administered, and will dissociate and rebind serum albumin rapidly. These characteristics will reduce rapid clearance of the drug (e.g., through the kidneys) but still provide efficient delivery and access to the drug target.

The antigen-binding fragment that binds serum albumin (e.g., dAb) generally binds with a KD of about 1 nM to about 500 μM, In some embodiments, the antigen-binding fragment binds serum albumin with a KD (KD=K_{off} (kd)/Kₒₙ (ka)) of about 10 to about 100 nM, or about 100 nM to about 500 nM, or about 500 nM to about 5 mM, as determined by surface plasmon resonance (e.g., using a BIACORE instrument). In particular embodiments, the drug conjugate, noncovalent drug conjugate or drug fusion comprises and antigen-binding fragment of an antibody (e.g., a dAb) that binds serum albumin (e.g., human serum albumin) with a KD of about 50 nM, or about 70 nM, or about 100 nM, or about 150 nM or about 200 nM, The improved pharmacokinetic properties (e.g., prolonged t½β increased AUC) of drug conjugates, noncovalent drug conjugates and drug fusions described herein may correlate with the affinity of the antigen-binding fragment that binds serum albumin. Accordingly, drug conjugates, noncovalent drug conjugates and drug fusions that have improved pharmacokinetic properties can generally be prepared using an antigen-binding fragment that binds serum albumin (e.g., human serum albumin) with high affinity (e.g., KD of about 500 nM or less, about 250 nM or less, about 100 nM or less, about 50 nM or less, about 10 nM or less, or about 1 nM or less, or about 100 pM or less).

Preferably, the drug that is conjugated or fused to the antigen-binding fragment that binds serum albumin, binds to its target (the drug target) with an affinity (KD) that is stronger than the affinity of the antigen-binding fragment for serum albumin and/or a K_{off} (kd) that is faster that the K_{off} of the antigen binding fragment for serum albumin, as measured by surface plasmon resonance (e.g., using a BIACORE instrument). For example, the drug can bind its target with an affinity that is about 1 to about 100000, or about 100 to about 100000, or about 1000 to about 100000, or about 10000 to about 100000 times stronger than the affinity of antigen-binding fragment that binds SA for SA. For example, the antigen-binding fragment of the antibody that binds SA can bind with an affinity of about 10 μM, while the drug binds its target with an affinity of about 100 pM.

In particular embodiments, the antigen-binding fragment of an antibody that binds serum albumin is a dAb that binds human serum albumin. For example, a V_{κ} dAb having an amino acid sequence selected from the group consisting of SEQ ID NO:10, SEQ ID NO:11, SEQ ID NO:12, SEQ ID NO:13, SEQ ID NO:14, SEQ ID NO:15, SEQ ID NO:24, SEQ ID NO:25 and SEQ ID NO:26, or a V_{H} dAb having an amino acid sequence selected from the group consisting of SEQ ID NO:16, SEQ ID NO:17, SEQ ID NO:18, SEQ ID NO:19, SEQ ID NO:20, SEQ ID NO:21, SEQ ID NO:22 and SEQ ID NO:23. In other embodiments, the antigen-binding fragment of an antibody that binds serum albumin is a dAb that binds human serum albumin and comprises the CDRs of any of the foregoing amino acid sequences. In other embodiments, the antigen-binding fragment of an antibody that binds serum albumin is a dAb that binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with SEQ ID NO:10, SEQ ID NO:11, SEQ ID NO:12, SEQ ID NO:13, SEQ ID NO:14, SEQ ID NO:15, SEQ ID NO:24, SEQ ID NO:25, SEQ ID NO:26, SEQ ID NO:16, SEQ ID NO:17, SEQ ID NO:18, SEQ ID NO:19, SEQ ID NO:20, SEQ ID NO:21, SEQ ID NO:22 or SEQ ID NO:23. Amino acid sequence identity is preferably determined using a suitable sequence alignment algorithm and default parameters, such as BLAST P (Karlin and Altschul, Proc. Natl. Acad. Sci. USA 57(6):2264-2268 (1990)).

### Drugs

Certain drug compositions of the invention (e.g., drug conjugates, noncovalent drug conjugates) can comprise any drug (e.g., small organic molecule, nucleic acid, polypeptide) that can be administered to an individual to produce a beneficial therapeutic or diagnostic effect, for example, through binding to and/or altering the function of a biological target molecule in the individual. Other drug compositions of the invention (e.g., drug fusions) can comprise a polypeptide or peptide drug. In preferred embodiments of drug fusions, the drug does not comprise an antibody chain or fragment of an antibody chain (e.g., V_{H}, V_{κ}, V_{λ}).

TNFR1 is a transmembrane receptor containing an extracellular region that binds ligand and an intracellular domain that lacks intrinsic signal transduction activity but can associate with signal transduction molecules. The complex of TNFR1 with bound TNF contains three TNFR1 chains and three TNF chains. (Banner et al., Cell, 73(3)431-445 (1993).) The TNF ligand is present as a trimer, which is bound by three TNFR1 chains. (Id.) The three TNFR1 chains are clustered closely together in the receptor-ligand complex, and this clustering is a prerequisite to TNFR1-mediated signal transduction. In fact, multivalent agents that bind TNFR1, such as anti-TNFR1 antibodies, can induce TNFR1 clustering and signal transduction in the absence of TNF and are commonly used as TNFR1 agonists. (See, e.g., Belka et al., EMBO, 14(6): 1156-1165 (1995); Mandik-Nayak et al., J. Immunol, 767:1920-1928 (2001).) Accordingly, multivalent agents that bind TNFR1, are generally not effective antagonists of TNFR1 even if they block the binding of TNFα to TNFR1.

The extracellular region of TNFR1 and other TNF receptor superfamily members contains a region referred to as the pre-ligand binding assembly domain or PLAD domain (amino acids 1-53 of SEQ ID NO:85 (human TNFR1); amino acids 1-53 of SEQ ID NO:86 (mouse TNFR1)) (The Government of the USA, WO 01/58953; U.S. Patent Application Publication No. 2003/0108992 A1, Deng et al., Nature Medicine, doi: 10.1038/nm 1304 (2005)).

The extracellular region of human (Homo sapiens) TNFR1 has the following amino acid sequence:

(SEQ ID NO:85)

LVPHLGDREKRDSVCPQGKYIHPQNNSICCTKCHKGTYLYNDCPGPGQDT
DCRECESGSFTASENHLRHCLSCKCRKEMGQVEISSCTVDRDTVCGCRKN
QYRHYWSENLFQCFNCSLCLNGTVHLSCQEKQNTVCTCHAGFFLRENECV
SCSNCKKSLECTKLCLPQIENVKGTEDSGTT.

The extracellular region of murine (Mus musculdus) TNFR1 has the following amino acid sequence:

(SEQ ID NO:86)

LVPSLGDREKRDSLCPQGKYVHSKNNSICCTKCHKGTYLVSDCPSPGRDT
VCRECEKGTFTASQNYLRQCLSCKTCRKEMSQVEISPCQADKDTVCGCKE
NQFQRYSETHFQCVDCSPCFNGTVTIPCKETQNTVCNCHAGFFLRESECV
PCSHCKKNEECMKLCLPPPLANVTNPQDSGTA

PLAD domains from a particular receptor bind to each other in vivo, and can prevent receptor activation in the presence of natural ligand. For example, the PLAD domain of TNFR1 will bind another PLAD domain of TNFR1 in vivo (e.g., TNFR1 expressed on the surface of a cell) and inhibit receptor clustering and subsequent signal transduction upon binding natural ligand.

The TNF receptor superfamily is an art recognized group of proteins that includes TNFR1 (p55, CD120a, p60, TNF receptor superfamily member 1A, TNFRSF1A), TNER2 (p75, p80, CD120b, TNF receptor superfamily member 1B, TNFRSF1B), CD (TNFRSF3, LTβR, TNFR2-RP, TNFR-RP, TNFCR, TNF-R-III), OX40 (TNFRSF4, ACT35, TXGP1L), CD40 (TNFRSF5, p50, Bp50), Fas (CD95, TNFRSF6, APO-1, APTI), DcR3 (TNFRSF6B), CD27 (TNFRSF7, Tp55, S152), CD30 (TNFRSF8, Ki-1, D1S166E), CD137 (TNFRSF9, 4-1BB, ILA), TRAILR-1 (TNFRSF10A, DR4, Apo2), TRAIL-R2 (TNFRSF10B, DR5, KILLER, TRICK2A, TRICKB), TRAILR3 (TNFRSF10C, DcR1, LIT, TRID), TRAILR4 (TNFRSF10D, DcR2, TRUNDD), RANK (TNFRSF11A), OPG (TNFRSF11B, OCIF, TR1), DR3 (TNFRSF12, TRAMP, WSL-1, LARD, WSL-LR, DDR3, TR3, APO-3), DR3L (TNFRSF12L), TAC1 (TNFRSF13B), BAFFR (TNFRSF13C), HVEM (TNFRSF14, ATAR, TR2, LIGHTR, HVEA), NGFR (TNFRSF16), BCMA (TNFRSF17, BCM), AITR (TNFRSF18, GITR), TNFRSF19, FLJ14993 (TNFRSF19L, RELT), DR6 (TNERSF21), SOBa (TNFRSF22, Tnfrh2, 2810028K06R1k), mSOB (THFRSF23, Tnfrh1),

Several PLAD domains are known in the art and other PLAD domains and functional variants of PLAD domains can be readily isolated and prepared using any suitable methods, such as the methods described in WO 01/58953; U.S. Patent Application Publication No. 2003/0108992 A1; Deng et al., Nature Medicine, doi: 10,1038/nm 1304 (2005). Many suitable methods for preparing polypeptides, protein fragments, and peptide variants, as well as suitable binding assays, such as the TNFR1 recepetor binding assay described herein are well-known and conventional in the art, Exemplary PLAD domains are presented in Table 8.

TABLE 8

| | |
|---|---|
| Receptor | PLAD Domain |

| | |
|---|---|
| TNFR1 | Cys Pro Gln Gly Lys Tyr Ile His Pro Gln |
| | Asn Asn Ser Ile Cys Cys Thr Lys Cys His |
| | Lys Gly Thr Tyr Leu Tyr Asn Asp Cys Pro |
| | Gly Pro Gly Gln Asp Thr Asp Cys |
| | (SEQ ID NO:87) |
| TNFR2 | Cys Arg Leu Arg Gln Tyr Tyr Asp Gln Thr |
| | Ala Gln Met Cys Cys Ser Lys Cys Ser Pro |
| | Gly Gln His Ala Lys Val Phe Cys Thr Lys |
| | Thr Ser Asp Thr Val Cys |
| | (SEQ ID NO:88) |
| FAS | Arg Leu Ser Ser Lys Ser Val Asn Ala Gln |
| | Val Thr Asp Ile Asn Ser Lys Gly Leu Glu |
| | Leu Arg Lys Thr Val Thr Thr Val Glu Thr |
| | Gln Asn Leu Glu Gly Leu His His Asp Gly |
| | Gln Phe Cys |
| | (SEQ ID NO:89) |
| FAS | Arg Leu Ser Ser Lys Ser Val Asn Ala Gln |
| | Val Thr Asp Ile Asn Ser Lys Gly Leu Glu |
| | Leu Arg Lys Thr Val Thr Thr Val Gln Thr |
| | Gln Asn Leu Glu Gly Leu His His Asp Gly |
| | Gln Phe Cys His Lys Pro Cys Pro Pro Gly |
| | Glu Arg Lys Ala Arg Asp Cys Thr Val Asn |
| | Gly Asp |
| | (SEQ ID NO:90) |
| LT βR | Cys Arg Asp Gln Glu Lys Glu Tyr Tyr Glu |
| | Pro Gln His Arg Ile Cys Cys Ser Arg Cys |
| | Pro Pro Gly Thr Tyr Val Ser Ala Lys Cys |
| | Ser Arg Ile Arg Asp Thr Val Cys |
| | (SEQ ID NO:91) |
| CD40 | Cys Arg Glu Lys Gln Tyr Leu Ile Asn Ser |
| | Gln Cys Cys Ser Leu Cys Gln Pro Gly Gln |
| | Lys Leu Val Ser Asp Cys Thr Glu Phe Thr |
| | Glu Thr Glu Cys |
| | (SEQ ID NO:92) |
| CD30 | Cys His Gly Asn Pro Ser His Tyr Tyr Asp |
| | Lys Ala Val Arg Arg Cys Cys Tyr Arg Cys |
| | Pro Met Gly Leu Phe Pro Thr Gln Gln Cys |
| | Pro Gln Arg Pro Thr Asp Cys Arg Lys Gln |
| | Cys |
| | (SEQ ID NO:93) |
| CD27 | Trp Trp Leu Cys Val Leu Gly Thr Leu Val |
| | Gly Leu Ser Ala Thr Pro Ala Pro Lys Ser |
| | Cys Pro Gln Arg His Tyr Trp Ala Gln Gly |
| | Lys Leu Cys Cys Gln Met |
| | (SEQ ID NO:94) |
| HVEM | Cys Lys Glu Asp Glu Tyr Pro Val Gly Ser |
| | Gln Cys Cys Pro Lys Cys Ser Pro Gly Tyr |
| | Arg Val Lys Glu Ala Cys Gly Glu Leu Thr |
| | Gly Thr Val Cys |
| | (SEQ ID NO;95) |
| OX40 | Val Gly Ala Arg Arg Leu Gly Arg Gly Pro |
| | Cys Ala Ala Leu Leu Leu Leu Gly Leu Gly |
| | Leu Ser Thr Val Thr Gly Leu His Cys Val |
| | Gly Asp Thr Tyr |
| | (SEQ ID NO:96) |
| DR4 | Ala Thr Ile Lys Leu His Asp Gln Ser Ile |
| | Gly Thr Gln Gln Trp Glu His Ser Pro Leu |
| | Gly Glu Leu Cys Pro Pro Gly Ser His Arg |
| | (SEQ ID NO:97) |

In some embodiments, the drug fusion or drug conjugate comprises a PLAD domain, such as a PLAD of TNFR1, TNFR2, FAS, LT βR, CD40, CD30, CD27, HVEM, OX40, DR4 or other TNF receptor superfamily member, or a functional variant of a PLAD domain. The functional variant of a PLAD domain can, for example, be a PLAD domain of TNFR1, TNFR2, FAS, LT βR, CD40, CD30, CD27, HVEM, OX40, or DR4, wherein one or more amino acids has been deleted, inserted or substituted, but that retains the ability to bind to the corresponding PLAD of TNFR1, TNFR2, FAS, LT βR, CD40, CD30, CD27, HVEM, OX40, or DR4. The amino acid sequence of a functional variant PLAD domain comprises a region of at least about 10 contiguous amino acids, at least about 15 contiguous amino acids, at least about 20 contiguous amino acids, at least about 25 contiguous amino acids, at least about 30 contiguous amino acids, at least about 35 contiguous amino acids, or at least about 40 contiguous amino acids that are the same as the amino acids in the amino acid sequence of the corresponding PLAD (e.g., PLAD of TNFR1, TNFR2, FAS, LT βR, CD40, CD30, CD27, HVEM, OX40, DR4). In addition, or alternatively, the amino acid sequence of a functional variant PLAD domain can be at least about 80%, at least about 85%, least about 90%, at least about 91%, at least about 92%, at least about 93%, at least about 94%, at least about 95%, at least about 96%, at least about 97%, at least about 98%, at least about 99% identical to the amino acid sequence of the corresponding PLAD (e.g., PLAD of TNFR1, TNFR2, FAS, LT PR, CD40, CD30, CD27, HVEM, OX40, or DR4).

In particular embodiments, the drug fusion or drug conjugate comprises a PLAD domain (e.g., PLAD of TNFR1, TNFR2, FAS, LT βR, CD40, CD30, CD27, HVEM, OX40, or DR4) or functional PLAD variant and a dAb that binds serum albumin or neonatal Fc receptor.

Additional suitable drugs, including polypeptide drugs, that can be used in the invention are disclosed in International Application No. PCT/GB2005/002163, filed in the name of Domantis Limited on May 31, 2005. The disclosure of suitable drugs disclosed in that application at pages 45 through 50 and Table 8. These drugs can be used in the invention, for example, to prepare a drug composition, fusion or conjugate that comprises a PLAD domain or functional variant of a PLAD domain, a polypeptide binding moiety that has a binding site that has bindng specificity for a polypeptide that enhances serum half-life in vivo, and another polypeptide drug. The teachings of International Application No. PCT/GB2005/002163 are incorporated herein by reference, in particular the teachings that relate to suitable drugs for use in the invention.

### Drug Fusions

The drug fusions of the invention are fusion proteins that comprise a continuous polypeptide chain, said chain comprising an antigen-binding fragment of an antibody that binds serum albumin as a first moiety, linked to a second moiety that is a polypeptide drug. The first and second moieties can be directly bonded to each other through a peptide bond, or linked through a suitable amino acid, or peptide or polypeptide linker. Additional moieties (e.g., third, fourth) and/or linker sequences can be present as appropriate. The first moiety can be in an N-terminal location, C-terminal location or internal relative to the second moiety (i.e., the polypeptide drug). In certain embodiments, each moiety can be present in more than one copy. For example, the drug fusion can comprise two or more first moieties each comprising an antigen-binding fragment of an antibody that binds serum albumin (e.g., a V_{H} that binds human serum albumin and a V_{L} that bind human serum albumin or two or more V_{H}_{^{S}} or V_{L}_{^{S}} that bind human serum albumin).

In some embodiments the drug fusion is a continuous polypeptide chain that has the formula:

a-(X)ₙ1-b-(Y)ₙ2-c-(Z)ₙ3-d or a-(Z)ₙ3-b-(Y)ₙ2-c-(X)ₙ1-d;

wherein X is a polypeptide drug that has binding specificity for a first target;

Y is a single chain antigen-binding fragment of an antibody that has binding specificity for serum albumin;

Z is a polypeptide drug that has binding specificity for a second target;

a, b, c and d are each independently absent or one to about 100 amino acid residues;

n1 is one to about 10;

n2 is one to about 10; and

n3 is zero to about 10,

with the proviso that when n1 and n2 are both one and n3 is zero, X does not comprise an antibody chain or a fragment of an antibody chain.

In one embodiment, neither X nor Z comprises an antibody chain or a fragment of an antibody chain. In one embodiment, n1 is one, n3 is one and n2 is two, three, four, five, six, seven, eight or nine. Preferably, Y is an immunoglobulin heavy chain variable domain (V_{H}) that has binding specificity for serum albumin, or an immunoglobulin light chain variable domain (V_{L}) that has binding specificity for serum albumin. More preferably, Y is a dAb (e.g., a V_{H}, V_{κ} or V_{λ}) that binds human serum albumin. In a particular embodiment, X or Z is human IL-1ra or a functional variant of human IL-1ra.

In certain embodiments, Y comprises an amino acid sequence selected from the group consisting of SEQ ID NO:10, SEQ ID NO:11, SEQ ID NO:12, SEQ ID NO:13, SEQ ID NO:14, SEQ ID NO:15, SEQ ID NO:24, SEQ ID NO:25 and SEQ ID NO:26. In other embodiments, Y comprises an amino acid sequence selected from the group consisting of SEQ ID NO:16, SEQ ID NO:17, SEQ ID NO:18, SEQ ED NO:19, SEQ ED NO:20, SEQ ED NO:21, SEQ ID NO:22 and SEQ ED NO:23.

In other embodiments, the drug fusion comprises moieties X′ and Y′, wherein X′ is a polypeptide drug, with the proviso that X′ does not comprise an antibody chain or a fragment of an antibody chain; and Y′ is a single chain antigen-binding fragment of an antibody that has binding specificity for serum albumin. Preferably, Y′ is an immunoglobulin heavy chain variable domain (V_{H}) that has binding specificity for serum albumin, or an immunoglobulin light chain variable domain (V_{L}) that has binding specificity for serum albumin. More preferably, Y′ is a dAb (e.g., a V_{H}, V_{κ} or V_{λ}) that binds human serum albumin. X′ can be located amino terminally to Y′, or Y′ can be located amino terminally to X′. In some embodiments, X′ and Y′ are separated by an amino acid, or by a peptide or polypeptide linker that comprises from two to about 100 amino acids. In a particular embodiment, X′ is human IL-1ra or a functional variant of human IL-1ra.

In certain embodiments, Y′ comprises an amino acid sequence selected from the group consisting of SEQ ED NO:10, SEQ ID NO:11, SEQ ID NO:12, SEQ ED NO:13, SEQ ED NO:14, SEQ ED NO:15, SEQ ED NO:24, SEQ ED NO:25 and SEQ ED NO:26. En other embodiments, Y′ comprises an amino acid sequence selected from the group consisting of SEQ ED NO:16, SEQ ED NO:17, SEQ ED NO:18, SEQ ED NO:19, SEQ ED NO:20, SEQ ED NO:21, SEQ ED NO:22 and SEQ ID NO:23.

In particular embodiments the drug fusion comprises a dAb that binds serum albumin and human IL-1ra (e.g., SEQ ED NO: 28). Preferably, the dAb binds human serum albumin and comprises human framework regions.

In other embodiments, the drug fusion or drug conjugate comprises a functional variant of human IL-1ra that has at least about 80%, or at least about 85%, or at least about 90%, or at least about 95%, or at least about 96%, or at least about 97%, or at least about 98%, or at least about 99% amino acid sequence identity with the mature 152 amino acid form of human IL-1ra and antagonizes human Interleukin-1 type 1 receptor. (See, Eisenberg et al., Nature 343:341-346 (1990).) The variant can comprise one or more additional amino acids (e.g., comprise 153 or 154 or more amino acids). The drug fusions of the invention can be produced using any suitable method. For example, some embodiments can be produced by the insertion of a nucleic acid encoding the drug fusion into a suitable expression vector. The resulting construct is then introduced into a suitable host cell for expression. Upon expression, fusion protein can be isolated or purified from a cell lysate or preferably from the culture media or periplasm using any suitable method. (See e.g., Current Protocols in Molecular Biology (Ausubel, F. M. et al., eds., Vol. 2, Suppl. 26, pp. 16.4.1-16.7.8 (1991)).

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., lac, tac, T3, T7 promoters for E. coli) and eucaryotic (e.g., simian virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin or replication. Genes encoding products which confer antibiotic or drug resistance are common selectable markers and may be used in procaryotic (e.g., lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Recombinant host cells that express a drug fusion and a method of preparing a drug fusion as described herein are provided. The recombinant host cell comprises a recombinant nucleic acid encoding a drug fusion. Drug fusions can be produced by the expression of a recombinant nucleic acid encoding the protein in a suitable host cell, or using other suitable methods. For example, the expression constructs described herein can be introduced into a suitable host cell, and the resulting cell can be maintained (e.g., in culture, in an animal) under conditions suitable for expression of the constructs. Suitable host cells can be prokaryotic, including bacterial cells such as E. coli, B. subtilis and or other suitable bacteria, eucaryotic, such as fungal or yeast cells (e.g., Pichia pastoris, Aspergillus species, Saccharomyces cerevisiae, Schizosaccharomyces pombe, Neurospora crassa), or other lower eucaryotic cells, and cells of higher eucaryotes such as those from insects (e.g., Sf9 insect cells (WO 94/26087 (O'Connor)) or mammals (e.g., COS cells, such as COS-1 (ATCC
Accession No. CRL-1650) and COS-7 (ATCC Accession No. CRL-1651), CHO (e.g., ATCC Accession No. CRL-9096), 293 (ATCC Accession No. CRL-1573), HeLa (ATCC Accession No. CCL-2), CV1 (ATCC Accession No. CCL-70), WOP (Dailey et al., J. Virol. 54:739-749 (1985)), 3T3, 293T (Pear et al., Proc. Natl. Acad. Sci. U.S.A., 90:8392-8396 (1993)), NSO cells, SP2/0, HuT 78 cells, and the like (see, e.g., Ausubel, F. M. et al., eds. Current Protocols in Molecular Biology, Greene Publishing Associates and John Wiley & Sons Inc., (1993)).

The invention also includes a method of producing a drug fusion, comprising maintaining a recombinant host cell of the invention under conditions appropriate for expression of a drug fusion. The method can further comprise the step of isolating or recovering the drug fusion, if desired. In another embodiment, the components of the drug fusion (e.g., dAb that binds human serum albumin and IL-1ra) are chemically assembled to created a continuous polypeptide chain.

### Conjugates

In another aspect, the invention provides conjugates comprising an antigen-binding fragment of an antibody that binds serum albumin that is bonded to a drug. Such conjugates include “drug conjugates,” which comprise an antigen-binding fragment of an antibody that binds serum albumin to which a drug is covalently bonded, and “noncovlaent drug conjugates,” which comprise an antigen-binding fragment of an antibody that binds serum albumin to which a drug is noncovalently bonded. Preferably, the conjugates are sufficiently stable so that the antigen-binding fragment of an antibody that binds serum albumin and drug remain substantially bonded (either covalently or noncovalently) to each other under in vivo conditions (e.g., when administered to a human). Preferably, no more than about 20%, no more than about 15%, no more than about 10%, no more than about 9%, no more than about 8%, no more than about 7%, no more than about 6%, no more than about 5%, no more than about 4%, no more than about 3%, no more than about 2%, no more than about 1% or substantially none of the conjugates dissociate or break down to release drug and antigen-binding fragment under in vivo conditions. For example, stability under “in vivo” conditions can be conveniently assessed by incubating drug conjugate or noncovalent drug conjugate for 24 hours in serum (e.g., human serum) at 37° C. In one example of such a method, equal amounts of a drug conjugate and the unconjugated drug are diluted into two different vials of serum. Half of the contents of each vial is immediately frozen at −20° C., and the other half incubated for 24 hours at 37° C. All four samples can then be analyzed using any suitable method, such as SDS-PAGE and/or Western blotting. Western blots can be probed using an antibody that binds the drug. All drug in the drug conjugate lanes will run at the size of the drug conjugate if there was no dissociation. Many other suitable methods can be used to assess stability under “in vivo” conditions, for example, by analyzing samples prepared as described above using suitable analytic methods, such as chromatography (e.g., gel filtration, ion exchange, reversed phase), ELISA, mass spectroscopy and the like.

### Drug Conjugates

In another aspect, the invention provides a drug conjugate comprising an antigen-binding fragment of an antibody that has binding specificity for serum albumin, and a drug that is covalently bonded to said antigen-binding fragment, with the proviso that the drug conjugate is not a single continuous polypeptide chain.

In some embodiments, the drug conjugate comprises an immunoglobulin heavy chain variable domain (V_{H}) that has binding specificity for serum albumin, or an immunoglobulin light chain variable domain (V_{L}) that has binding specificity for serum albumin, and a drug that is covalently bonded to said V_{H} or V_{L}, with the proviso that the drug conjugate is not a single continuous polypeptide chain. Preferably the drug conjugate comprises a single V_{H} that binds serum albumin or a single V_{L} that binds serum albumin. In certain embodiments, the drug conjugate comprises a V_{κ} dAb that binds human serum albumin and comprises an amino acid sequence selected from the group consisting of SEQ ID NO:10, SEQ ID NO:11, SEQ ID NO:12, SEQ ID NO:13, SEQ ID NO:14, SEQ ED NO:15, SEQ ID NO:24, SEQ ID NO:25 and SEQ ID NO:26. In other embodiments, the drug conjugate comprises a V_{H} dAb that binds human serum albumin and comprises an amino acid sequence selected from the group consisting of SEQ ID NO:16, SEQ ED NO:17, SEQ ID NO:18, SEQ ID NO:19, SEQ ID NO:20, SEQ ED NO:21, SEQ ID NO:22 and SEQ ID NO:23.

The drug conjugates can comprise any desired drug and can be prepared using any suitable methods. For example, the drug can be bonded to the antigen-binding fragment of an antibody that binds serum albumin directly or indirectly through a suitable linker moiety at one or more positions, such as the amino-terminus, the carboxyl-terminus or through amino acid side chains. In one embodiment, the drug conjugate comprises a dAb that binds human serum albumin and a polypeptide drug (e.g., human IL-1ra or a functional variant of human IL-1ra), and the amino-terminus of the polypeptide drug (e.g., human IL-1ra or a functional variant of human IL-1ra) is bonded to the carboxyl-terminus of the dAb directly or through a suitable linker moiety. In other embodiments, the drug conjugate comprises a dAb that binds human serum albumin and two or more different drugs that are covalently bonded to the dAb. For example, a first drug can be covalently bonded (directly or indirectly) to the carboxyl terminus of the dAb and a second drug can be covalently bonded (directly or indirectly) to the amino-terminus or through a side chain amino group (e.g., ε amino group of lysine). Such drug conjugates can be prepared using well-known methods of selective coupling. (See, e.g., Hermanson, G. T., Bioconjugate Techniques, Academic Press: San Diego, Calif. (1996).)

A variety of methods for conjugating drugs to an antigen-binding fragment of an antibody that has binding specificity for serum albumin can be used. The particular method selected will depend on the drug to be conjugated. If desired, linkers that contain terminal functional groups can be used to link the antigen-binding fragment and the drug. Generally, conjugation is accomplished by reacting a drug that contains a reactive functional group (or is modified to contain a reactive functional group) with a linker or directly with an antigen-binding fragment of an antibody that binds serum albumin. Covalent bonds form by reacting a drug that contains (or is modified to contain) a chemical moiety or functional group that can, under appropriate conditions, react with a second chemical group thereby forming a covalent bond. If desired, a suitable reactive chemical group can be added to the antigen-binding fragment or to a linker using any suitable method. (See, e.g., Hermanson, G. T., Bioconjugate Techniques, Academic Press: San Diego, Calif. (1996).) Many suitable reactive chemical group combinations are known in the art, for example an amine group can react with an electrophilic group such as tosylate, mesylate, halo (chloro, bromo, fluoro, iodo), N-hydroxysuccinimidyl ester (NHS), and the like. Thiols can react with maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

In some embodiments, the antigen-binding fragment of an antibody that has binding specificity for serum albumin is bonded to a drug by reaction of two thiols to form a disulfide bond. In other embodiments, the antigen-binding fragment of an antibody that has binding specificity for serum albumin is bonded to a drug by reaction of an isothiocyanate group and a primary amine to produce an isothiourea bond.

Suitable linker moieties can be linear or branched and include, for example, tetraethylene glycol, C₂-C₁₂ alkylene, —NH—(CH₂)ₚ—NH— or —(CH₂)ₚ—NH— (wherein p is one to twelve), —CH₂—O—CH₂—CH₂—O—CH₂—CH₂—O—CH—NH—, a polypeptide chain comprising one to about 100 (preferably one to about 12) amino acids and the like.

### Noncovalent Drug Conjugates

Some noncovalent bonds (e.g., hydrogen bonds, van der Waals interactions) can produce stable, highly specific intermolecular connections. For example, molecular recognition interactions achieved through multiple noncovalent bonds between complementary binding partners underlie many important biological interactions, such as the binding of enzymes to their substrates, the recognition of antigens by antibodies, the binding of ligands to their receptors, and stabilization of the three dimensional structure of proteins and peptide. Accordingly, such weak noncovalent interactions (e.g., hydrogen bonding, van Der Waals interactions, electrostatic interactions, hydrophobic interactions and the like) can be utilized to bind a drug to the antigen-binding fragment of an antibody that has binding specificity for serum albumin.

Preferably, the noncovalent bond linking the antigen-binding fragment and drug be of sufficient strength that the antigen-binding fragment and drug remain substantially bonded to each under in vivo conditions (e.g., when administered to a human). Generally, the noncovalent bond linking the antigen-binding fragment and drug has a strength of at least about 10¹⁰ M⁻¹. In preferred embodiments, the strength of the noncovalent bond is at least about 10¹¹M⁻¹, at least about 10¹²M⁻¹, at least about 10¹³M⁻¹ at least about 10¹⁴M⁻¹ or at least about 10¹⁵M⁻¹. The interactions between biotin and avidin and between biotin and streptavidin are known to be very efficient and stable under many conditions, and as described herein noncovalent bonds between biotin and avidin or between biotin and streptavidin can be used to prepare a noncovalent drug conjugate of the invention.

The noncovalent bond can be formed directly between the antigen-binding fragment of an antibody that has a specificity for serum albumin and drug, or can be formed between suitable complementary binding partners (e.g., biotin and avidin or streptavidin) wherein one partner is covalently bonded to drug and the complementary binding partner is covalently bonded to the antigen-binding fragment. When complementary binding partners are employed, one of the binding partners can be covalently bonded to the drug directly or through a suitable linker moiety, and the complementary binding partner can be covalently bonded to the antigen-binding fragment of an antibody that binds serum albumin directly or through a suitable linker moiety.

Complementary binding partners are pairs of molecules that selectively bind to each other. Many complementary binding partners are known in the art, for example, antibody (or an antigen-binding fragment thereof) and its cognate antigen or epitope, enzymes and their substrates, and receptors and their ligands. Preferred complementary binding partners are biotin and avidin, and biotin and streptavidin.

Direct or indirect covalent bonding of a member of a complementary binding pair to an antigen-binding fragment that has binding specificity for serum albumin or a drug can be accomplished as described above, for example, by reacting a complementary binding partner that contains a reactive functional group (or is modified to contain a reactive functional group) with an antigen-binding fragment of an antibody that binds serum albumin, with or without the use of a linker. The particular method selected will depend on the compounds (e.g., drug, complementary binding partner, antigen-binding fragment of an antibody that binds serum albumin) to be conjugated. If desired, linkers (e.g., homobifunctional linkers, heterobifunctional linkers) that contain terminal reactive functional groups can be used to link the antigen-binding fragment and/or the drug to a complementary binding partner. In one embodiment, a heterobifunctional linker that contains two distinct reactive moieties can be used. The heterobifunctional linker can be selected so that one of the reactive moieties will react with the antigen-binding fragment of an antibody that has binding specificity for serum albumin or the drug, and the other reactive moiety will react with the complementary binding partner. Any suitable linker (e.g., heterobifunctional linker) can be used and many such linkers are known in the art and available for commercial sources (e.g., Pierce Biotechnology, Inc., IL).

### Compositions and Therapeutic and Diagnostic Methods

Compositions comprising drug compositions of the invention (e.g., drug conjugates, noncovalent drug conjugates, drug fusions), including pharmaceutical or physiological compositions (e.g., for human and/or veterinary administration) are provided. Pharmaceutical or physiological compositions comprise one or more drug compositions (e.g., drug conjugate, noncovalent drug conjugate, drug fusion), and a pharmaceutically or physiologically acceptable carrier. Typically, these carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates. Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compositions can comprise a desired amount of drug composition (e.g., drug conjugate, noncovalent drug conjugate, drug fusion). For example the compositions can comprise about 5% to about 99% drug conjugate, noncovalent drug conjugate or drug fusion by weight. In particular embodiments, the composition can comprise about 10% to about 99%, or about 20% to about 99%, or about 30% to about 99% or about 40% to about 99%, or about 50% to about 99%, or about 60% to about 99%, or about 70% to about 99%, or about 80% to about 99%, or about 90% to about 99%, or about 95% to about 99% drug composition (e.g., drug conjugate, noncovalent drug conjugate, drug fusion), by weight. In one example, the composition is freeze dried (lyophilized).

The drug compositions (e.g., drug conjugates, noncovalent drug conjugates, drug fusions), described herein will typically find use in preventing, suppressing or treating inflammatory states (e.g., acute and/or chronic inflammatory diseases), such as chronic obstructive pulmonary disease (e.g., chronic bronchitis, chronic obstructive bronchitis, emphysema), allergic hypersensitivity, cancer, bacterial or viral infection, pneumonia, such as bacterial pneumonia (e.g., Staphylococcal pneumonia)), autoimmune disorders (which include, but are not limited to, Type I diabetes, multiple sclerosis, arthritis (e.g., osteoarthritis, rheumatoid arthritis, juvenile rheumatoid arthritis, psoriatic arthritis, lupus arthritis, spondylarthropathy (e.g., ankylosing spondylitis)), systemic lupus erythematosus, inflammatory bowel disease (e.g., Crohn's disease, ulcerative colitis), Behcet's syndrome and myasthenia gravis), endometriosis, psoriasis, abdominal adhesions (e.g., post abdominal surgery), asthma, and septic shock. The drug compositions (e.g., drug conjugates, noncovalent drug conjugates, drug fusions), described herein can be used for preventing, suppressing or treating pain, such as chronic or acute traumatic pain, chronic or acute neuropathic pain, acute or chronic musculoskeletal pain, chronic or acute cancer pain and the like. The drug compositions (e.g., drug conjugates, noncovalent drug conjugates, drug fusions), described herein can also be administered for diagnostic purposes.

The drug compositions (e.g., drug conjugates, noncovalent drug conjugates, drug fusions) described herein are also suitable for use in preventing, suppressing or treating lung inflammation, chronic obstructive respiratory disease (e.g., chronic bronchitis, chronic obstructive bronchitis, emphysema), asthma (e.g., steroid resistant asthma), pneumonia (e.g., bacterial pneumonia, such as Staphylococcal pneumonia), hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease (IPD), influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X (eosinophilic granuloma), pulmonary hypertension, pulmonary nocardiosis, pulmonary tuberculosis, pulmonary veno-occlusive disease, rheumatoid lung disease, sarcoidosis, Wegener's granulomatosis, and non-small cell lung carcinoma.

The drug compositions (e.g., drug conjugates, noncovalent drug conjugates, drug fusions) described herein are also suitable for use in preventing, suppressing or treating treat influenza, RSV-associated respiratory disease and viral lung (respiratory) disease.

The drug compositions (e.g., drug conjugates, noncovalent drug conjugates, drug fusions) described herein are also suitable for use in preventing, suppressing or treating osteoarthritis or inflammatory arthritis. “Inflammatory arthritis” refers to those diseases of joints where the immune system is causing or exacerbating inflammation in the joint, and includes rheumatoid arthritis, juvenile rheumatoid arthritis, and spondyloarthropathies, such as ankylosing spondylitis, reactive arthritis, Reiter's syndrome, psoriatic arthritis, psoriatic spondylitis, enteropathic arthritis, enteropathic spondylitis, juvenile-onset spondyloarthropathy and undifferentiated spondyloarthropathy. Inflammatory arthritis is generally characterized by infiltration of the synovial tissue and/or synovial fluid by leukocytes.

Cancers that can be prevented, suppressed or treated using the drug compositions (e.g., drug conjugates, noncovalent drug conjugates, drug fusions), described herein include lymphomas (e.g., B cell lymphoma, acute myeloid lymphoma, Hodgkin's lymphoma, non-Hodgkin's lymphoma), myelomas (e.g., multiple myeloma), lung cancer (e.g., small cell lung carcinoma, non-small cell lung carcinoma), colorectal cancer, head and neck cancer, pancreatic cancer, liver cancer, stomach cancer, breast cancer, ovarian cancer, bladder cancer, leukemias (e.g., acute myelogenous leukemia, chronic myelogenous leukemia, acute lymphocytic leukemia, chronic lymphocytic leukemia), adenocarcinomas, renal cancer, hematopoetic cancers (e.g., myelodysplastic syndrome, myeloproliferative disorder e.g., polycyinemia vera, essential (or primary) thrombocythemia, idiopathic myelofibrosis), and the like.

The drug compositions (e.g., drug conjugates, noncovalent drug conjugates, drug fusions) described herein are also suitable for use in preventing, suppressing or treating endometriosis, fibrosis, infertility, premature labour, erectile dysfunction, osteoporosis, diabetes (e.g., type II diabetes), growth disorder, HIV infection, respiratory distress syndrome, tumors and bedwetting.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of drug compositions (e.g., drug conjugates, noncovalent drug conjugates, drug fusions) in protecting against or treating the disease are available. Methods for the testing of systemic lupus erythematosus (SLE) in susceptible mice are known in the art (Knight et al. (1978) J. Exp. Med., 147:1653; Reinersten et al. (1978) New Eng. J. Med., 299:515). Myasthenia Gravis (MG) is tested in SJL/J female mice by inducing the disease with soluble AchR protein from another species (Lindstrom et al. (1988) Adv. Immunol., 42:233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331:171). Effectiveness for treating osteoarthritis can be assessed in a murine model in which arthritis is induced by intra-articular injection of collagenase (Blom, A. B. et al., Osteoarthritis Cartilage 12:627-635 (2004). Thyroiditis is induced in mice by administration of thyroglobulin as described (Maron et al. (1980) J. Exp. Med., 152:1115). Insulin dependent diabetes mellitus (IDDM) occurs naturally or can be induced in certain strains of mice such as those described by Kanasawa et al. (1984) Diabetologia, 27:113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, New York, pp. 179-213; McFarlin et al. (1973) Science, 179:478: and Satoh et al. (1987) J. Immunol., 138: 179).

The drug compositions (e.g., drug conjugates, noncovalent drug conjugates, drug fusions) of the present invention may be used as separately administered compositions or in conjunction with other agents. These can include various immunotherapeutic drugs, such as cylcosporine, methotrexate, adriamycin or cisplatinum, immunotoxins and the like. For example, when the drug compositions (e.g., drug conjugates, noncovalent drug conjugates, drug fusions) is administered to prevent, suppress or treat lung inflammation or a respiratory disease, it can be administered in conjunction with phosphodiesterase inhibitors (e.g., inhibitors of phosphodiesterase 4), bronchodilators (e.g., beta2-agonists, anticholinergerics, theophylline), short-acting beta-agonists (e.g., albuterol, salbutamol, bambuterol, fenoterol, isoetherine, isoproterenol, levalbuterol, metaproterenol, pirbuterol, terbutaline and tornlate), long-acting beta-agonists (e.g., formoterol and salmeterol), short acting anticholinergics (e.g., ipratropium bromide and oxitropium bromide), long-acting anticholinergics (e.g., tiotropium), theophylline (e.g. short acting formulation, long acting formulation), inhaled steroids (e.g., beclomethasone, beclometasone, budesonide, flunisolide, fluticasone propionate and triamcinolone), oral steroids (e.g., methylprednisolone, prednisolone, prednisolon and prednisone), combined short-acting beta-agonists with anticholinergics (e.g., albuterol/salbutamol/ipratopium, and fenoterol/ipratopium), combined long-acting beta-agonists with inhaled steroids (e.g., salmeterol/fluticasone, and formoterol/budesonide) and mucolytic agents (e.g., erdosteine, acetylcysteine, bromheksin, carbocysteine, guiafenesin and iodinated glycerol.

For example, when the drug compositions (e.g., drug conjugates, noncovalent drug conjugates, drug fusions) is administered to prevent, suppress or treat arthritis (e.g., inflammatory arthritis (e.g., rheumatoid arthritis)), it can be administered in conjunction with a disease modifying anti-rheumatic agent (e.g., methotrexate, hydroxychloroquine, sulfasalazine, leflunomide, azathioprine, D-penicillamine, gold (oral or intramuscular), minocycline, cyclosporine, staphylococcal protein A), nonsteroidal anti-inflammatory agent (e.g., COX-2 selective NSAIDS such as rofecoxib), salicylates, glucocorticoids (e.g., prednisone) and analgesics.

Pharmaceutical compositions can include “cocktails” of various cytotoxic or other agents in conjunction with the drug composition (e.g., drug conjugate, noncovalent drug conjugate, drug fusion) of the present invention, or combinations of drug compositions (e.g., drug conjugates, noncovalent drug conjugates, drug fusions) according to the present invention comprising different drugs.

The drug compositions (e.g., drug conjugates, noncovalent drug conjugates, drug fusions) can be administered to any individual or subject in accordance with any suitable techniques. A variety of routes of administration are possible including, for example, oral, dietary, topical, transdermal, rectal, parenteral (e.g., intravenous, intraarterial, intramuscular, subcutaneous, intradermal, intraperitoneal, intrathecal, intraarticular injection), and inhalation (e.g., intrabronchial, intranasal or oral inhalation, intranasal drops) routes of administration, depending on the drug composition and disease or condition to be treated. Administration can be local or systemic as indicated. The preferred mode of administration can vary depending upon the drug composition (e.g., drug conjugate, noncovalent drug conjugate, drug fusion) chosen, and the condition (e.g., disease) being treated. The dosage and frequency of administration will depend on the age, sex and condition of the patient, concurrent administration of other drugs, counterindications and other parameters to be taken into account by the clinician. A therapeutically effective amount of a drug composition (e.g., drug conjugate, noncovalent drug conjugate, drug fusion) is administered. A therapeutically effective amount is an amount sufficient to achieve the desired therapeutic effect, under the conditions of administration.

The term “subject” or “individual” is defined herein to include animals such as mammals, including, but not limited to, primates (e.g., humans), cows, sheep, goats, horses, dogs, cats, rabbits, guinea pigs, rats, mice or other bovine, ovine, equine, canine, feline, rodent or murine species.

The drug composition (e.g., drug conjugate, noncovalent drug conjugate, drug fusion) can be administered as a neutral compound or as a salt. Salts of compounds (e.g., drug compositions, drug conjugates, noncovalent drug conjugates, drug fusions) containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium and the like.

The invention also provides a kit for use in administering a drug composition (e.g., drug conjugate, noncovalent drug conjugate, drug fusion) to a subject (e.g., patient), comprising a drug composition (e.g., drug conjugate, noncovalent drug conjugate, drug fusion), a drug delivery device and, optionally, instructions for use. The drug composition (e.g., drug conjugate, noncovalent drug conjugate, drug fusion) can be provided as a formulation, such as a freeze dried formulation. In certain embodiments, the drug delivery device is selected from the group consisting of a syringe, an inhaler, an intranasal or ocular administration device (e.g., a mister, eye or nose dropper), and a needleless injection device.

The drug composition (e.g., drug conjugate, noncovalent drug conjugate, drug fusion) of this invention can be lyophilized for storage and reconstituted in a suitable carrier prior to use. Any suitable lyophilization method (e.g., spray drying, cake drying) and/or reconstitution techniques can be employed. It will be appreciated by those skilled in the art that lyophilisation and reconstitution can lead to varying degrees of antibody activity loss (e.g., with conventional immunoglobulins, IgM antibodies tend to have greater activity loss than IgG antibodies) and that use levels may have to be adjusted to compensate. In a particular embodiment, the invention provides a composition comprising a lyophilized (freeze dried) drug composition (e.g., drug conjugate, noncovalent drug conjugate, drug fusion) as described herein. Preferably, the lyophilized (freeze dried) drug composition (e.g., drug conjugate, noncovalent drug conjugate, drug fusion) loses no more than about 20%, or no more than about 25%, or no more than about 30%, or no more than about 35%, or no more than about 40%, or no more than about 45%, or no more than about 50% of its activity (e.g., binding activity for serum albumin) when rehydrated. Activity is the amount of drug composition (e.g., drug conjugate, noncovalent drug conjugate, drug fusion) required to produce the effect of the drug composition before it was lyophilized. For example, the amount of drug conjugate or drug fusion needed to achieve and maintain a desired serum concentration for a desired period of time. The activity of the drug composition (e.g., drug conjugate, noncovalent drug conjugate, drug fusion) can be determined using any suitable method before lyophilization, and the activity can be determined using the same method after rehydration to determine amount of lost activity.

Compositions containing the drug composition (e.g., drug conjugate, noncovalent drug conjugate, drug fusion) or a cocktail thereof can be administered for prophylactic and/or therapeutic treatments. In certain therapeutic applications, an amount sufficient to achieve the desired therapeutic or prophylactic effect, under the conditions of administration, such as at least partial inhibition, suppression, modulation, killing, or some other measurable parameter, of a population of selected cells is defined as a “therapeutically-effective amount or dose.” Amounts needed to achieve this dosage will depend upon the severity of the disease and the general state of the patient's own immune system and general health, but generally range from about 10 μg/kg to about 80 mg/kg, or about 0.005 to 5.0 mg of drug conjugate or drug fusion per kilogram of body weight, with doses of 0.05 to 2.0 mg/kg/dose being more commonly used. For example, a drug composition (e.g., drug fusion, drug conjugate, noncovalent drug conjugate) of the invention can be administered daily (e.g., up to four administrations per day), every two days, every three days, twice weekly, once weekly, once every two weeks, once a month, or once every two months, at a dose of, for example, about 10 μg/kg to about 80 mg/kg, about 100 μg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 10 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.

For prophylactic applications, compositions containing the drug composition (e.g., drug conjugate, noncovalent drug conjugate, drug fusion) or cocktails thereof may also be administered in similar or slightly lower dosages. A composition containing a drug composition (e.g., drug conjugate, noncovalent drug conjugate, drug fusion) according to the present invention may be utilised in prophylactic and therapeutic settings to aid in the alteration, inactivation, killing or removal of a select target cell population in a mammal.

### EXAMPLES

Interleukin 1 receptor antagonist (IL1-ra) is an antagonist that blocks the biologic activity of IL-1 by competitively inhibiting IL-1 binding to the interleukin-1 type 1 receptor (IL-1R1). IL-1 production is induced in response to inflammatory stimuli and mediates various physiologic responses including inflammatory and immunological responses. IL-1 has a range of activities including cartilage degredation and stimulation of bone resorption. In rheumatoid arthritis patients, the amount of locally produced IL-1 is elevated and the levels of naturally occurring IL1-ra are insufficient to compete with these abnormally increased amounts. There are several treatments available for RA including disease modifying antirheumatic drugs (DMARDS) such as methotrexate, and biologies such as KINERET® (anakinra, Amgen Inc).

KINERET® (anakinra, Amgen Inc) is a recombinant, nonglycosylated form of the human interleukin-1 receptor antagonist which consists of 153 amino acids and has a molecular weight of 17.3 kilodaltons. (The amino acid sequence of KINERET® (anakinra, Amgen Inc) corresponds to the 152 amino acids in naturally occurring IL-1ra and an additional N-terminal methionine.) KINERET® (anakinra, Amgen Inc) is indicated for the reduction in signs and symptoms of moderate to severe rheumatoid arthritis in patients 18 years of age or older who have failed one or more DMARDs. Dosage is a single use daily subcutaneous injection of 100 mgs of drug. The T_{β}½ is 4-6 hours and 71% of patients develop injection site reactions in 14-28 days.

Here we demonstrate that linking a therapeutic polypeptide to a serum-albumin binding dAb results in a compound which (i) has activity similar to the therapeutic polypeptide alone and (ii) also binds serum albumin. Furthermore, the present invention provides a method to create a long serum half-life version of the therapeutic polypeptide. For example, we have linked a serum albumin binding dAb to IL1-ra which results in a compound of longer serum half-life than IL1-ra alone.

### Example 1

### Selection of Domain Antibodies that Bind Mouse, Rat and Human Serum Albumin

This example explains a method for making a single domain antibody (dAb) directed against serum albumin. Selection of dAbs against mouse serum albumin (MSA), human serum albumin (HSA) and rat serum albumin (RSA) is described.

The dAbs against mouse serum albumin were selected as described in WO 2004/003019 A2. Three human phage display antibody libraries were used. Each library was based on a single human framework for V_{H} (V3-23/DP47 and J_{H}4b) or V_{κ} (o12/o2/DPK9 and J_{κ1}) with side chain diversity encoded by NNK codons incorporated in complementarity determining regions (CDR1, CDR2 and CDR3).

### Library 1 (V_{H}):

Diversity at positions: H30, H31, H33, H35, H50, H52, H52a, H53, H55, H56, H58, H95, H97, H98.
Library size: 6.2×10⁹

### Library 2 (V_{H}):

Diversity at positions: H30, H31, H33, H35, H50, H52, H52a, H53, H55, H56, H58, H95, H97, H98, H99, H100, H100A, H100B.
Library size: 4.3×10⁹

### Library 3 (V_{κ}):

Diversity at positions: L30, L31, L32, L34, L50, L53, L91, L92, L93, L94, L96

Library size: 2×10⁹
The V_{H} and V_{κ} libraries had been preselected for binding to generic ligands protein A and protein L respectively so that the majority of clones in the selected libraries were functional. The sizes of the libraries shown above correspond to the sizes after preselection.

Two rounds of selection were performed on serum albumin using each of the libraries separately. For each selection, antigen was coated on immunotube (nunc) in 4 mL of PBS at a concentration of 100 μg/ml. In the first round of selection, each of the three libraries was panned separately against HSA (Sigma) or MSA (Sigma). In the second round of selection, phage from each of the six first round selections was panned against (i) the same antigen again (eg 1st round MSA, 2nd round MSA) and (ii) against the reciprocal antigen (eg 1^{st} round MSA, 2nd round HSA) resulting in a total of twelve 2nd round selections. In each case, after the second round of selection 48 clones were tested for binding to HSA and MSA. Soluble dAb fragments were produced as described for scFv fragments by Harrison et al., Methods Enzymol. 1996; 267: 83-109 and standard ELISA protocol was followed (Hoogenboom et al. (1991) Nucleic Acids Res., 19:4133) except that 2% tween PBS was used as a blocking buffer and bound dAbs were detected with either protein L-HRP (Sigma) (for the VκS) and protein A-HRP (Amersham Pharmacia Biotech) (for the V_{H}_{^{S}}).

dAbs that gave a signal above background indicating binding to MSA, HSA or both were tested in ELISA insoluble form for binding to plastic alone but all were specific for serum albumin. Clones were then sequenced (see Table 1) revealing that 21 unique dAb sequences had been identified. The minimum similarity (at the amino acid level) between the V_{κ} dAb clones selected was 86.25% ((69/80) X100; the result when all the diversified residues are different, e.g., clones 24 and 34). The minimum similarity between the V_{H} dAb clones selected was 94% ((127/136) X100).

Next, the serum albumin binding dAbs were tested for their ability to capture biotinylated antigen from solution. ELISA protocol (as above) was followed except that ELISA plate was coated with 1 μg/ml protein L (for the V_{κ} clones) and 1 μg/ml protein A (for the V_{H} clones). Soluble dAb was captured from solution as in the protocol and detection was with biotinylated MSA or HSA and streptavidin HRP. The biotinylated MSA and HSA had been prepared according to the manufacturer's instructions, with the aim of achieving an average of 2 biotins per serum albumin molecule. Twenty four clones were identified that captured biotinylated MSA from solution in the ELISA. Two of these (clones 2 and 38 below) also captured biotinylated HSA. Next, the dAbs were tested for their ability to bind MSA coated on a CM5 biacore chip. Eight clones were found that bound MSA on the biacore.

dAbs against human serum albumin and rat serum albumin were selected as previously described for the anti-MSA dAbs except for the following modifications to the protocol: The phage library of synthetic V_{H} domains was the library 4G, which is based on a human V_{H}3 comprising the DP47 germline gene and the J_{H}4 segment. The diversity at the following specific positions was introduced by mutagenesis (using NNK codons; numbering according to Kabat) in CDR1: 30, 31, 33, 35; in CDR2: 50, 52, 52a, 53, 55, 56; and in CDR3: 4-12 diversified residues: e.g. H95, H96, H97, and H98 in 4G HI 1 and H95, H96, H97, H98, H99, H100, H100a, H100b, H100c, H100d, H100e and H100f in 4G H19. The last three CDR3 residues are FDY so CDR3 lengths vary from 7-15 residues. The library comprises >1×10¹⁰ individual clones.

A subset of the V_{H} and V_{κ} libraries had been preselected for binding to generic ligands protein A and protein L respectively so that the majority of clones in the unselected libraries were functional. The sizes of the libraries shown above correspond to the sizes after preselection.

Two rounds of selection were performed on rat and human serum albumin using subsets of the V_{H} and V_{κ} libraries separately. For each selection, antigen was either (i) coated on immunotube (nunc) in 4 ml of PBS at a concentration of 100 μg/ml or (ii) bitotinylated and then used for soluble selection followed by capture on streptavidin beads (in the 1^{st} round) and neutravidin beads (in the 2^{nd} round). (See Table 1 for details of the selection strategy used to isolate each clone.)

In each case, after the second round of selection 24 phage clones were tested for binding to HSA or RSA.

If a significant proportion of the clones in one of the selections were positive in the phage ELISA, then DNA from this selection was cloned into an expression vector for production of soluble dAb, and individual colonies were picked. Soluble dAb fragments were produced as described for scFv fragments by Harrison et al (Methods Enzymol. 1996; 267:83-109) and standard ELISA protocol was followed (Hoogenboom et al. (1991) Nucleic Acids Res., 19: 4133) except that 2% TWEEN PBS was used as a blocking buffer and bound dAbs were detected with anti-myc-HRP Clones that were positive in ELISA were then screened for binding to MSA, RSA or HSA using a BIACORE surface plasmon resonance instrument (Biacore AB). dAbs which bound to MSA, RSA or HSA were further analysed. Clones were then sequenced and unique dAb sequences identified.

TABLE 1
Selection protocols for dAbs that bind serum albumin

| | | | | Biacore |
|---|---|---|---|---|
| dAb | Library | R1 selection | R2 selection | binding |
| DOM7r-1 | 4G Vκ | 10 μg/ml tube | 10 μg/ml tube | RSA |
| | | RSA | RSA | |
| DOM7r-3 | 4G Vκ | 10 μg/ml tube | 10 μg/ml tube | RSA |
| | | RSA | RSA | |
| DOM7r-4 | 4G Vκ | 10 μg/ml tube | 10 μg/ml tube | RSA, MSA |
| | | RSA | RSA | |
| DOM7r-5 | 4G Vκ | 10 μg/ml tube | 10 μg/ml tube | RSA |
| | | RSA | RSA | |
| DOM7r-7 | 4G Vκ | 10 μg/ml tube | 10 μg/ml tube | RSA, MSA |
| | | RSA | RSA | |
| DOM7r-8 | 4G Vκ | 10 μg/ml tube | 10 μg/ml tube | RSA, MSA |
| | | RSA | RSA | |
| DOM7h-1 | 4G Vκ | 10 μg/ml tube | 10 μg/ml tube | HSA |
| | | HSA | HSA | |
| DOM7h-2 | 4G Vκ | Soluble 100 nM | Soluble 50 nM | HSA |
| | | HSA | HSA | |
| DOM7h-3 | 4G Vκ | 10 μg/ml tube | 10 μg/ml tube | — |
| | | HSA | HSA | |
| DOM7h-4 | 4G Vκ | 10 μg/ml tube | 10 μg/ml tube | — |
| | | HSA | HSA | |
| DOM7h-6 | 4G Vκ | | | |
| DOM7h-7 | 4G Vκ | | | |
| DOM7h-8 | 4G Vκ | Soluble 200 nM | Soluble 50 nM | HSA, RSA, |
| | | HAS | RSA | MSA |
| DOM7r-13 | 4G Vκ | Soluble 200 nM | Soluble 50 nM | RSA, MSA |
| | | HAS | RSA | |
| DOM7r-14 | 4G Vκ | Soluble 200 nM | Soluble 50 nM | RSA, MSA |
| | | HAS | RSA | |
| DOM7h-21 | 4G VH | 100 μg/ml HSA | 100 μg/ml HSA | HSA |
| | | tube | tube | |
| DOM7h-22 | 4G VH | 100 μg/ml HSA | 100 μg/ml HSA | HSA |
| | | tube | tube | |
| DOM7h-23 | 4G VH | 100 μg/ml HSA | 100 μg/ml HSA | HSA |
| | | tube | tube | |
| DOM7h-24 | 4G VH | 100 μg/ml HSA | 100 μg/ml HSA | HSA |
| | | tube | tube | |
| DOM7h-25 | 4G VH | 100 μg/ml HSA | 100 μg/ml HSA | HSA |
| | | tube | tube | |
| DOM7h-26 | 4G VH | 100 μg/ml HSA | 100 μg/ml HSA | HSA |
| | | tube | tube | |
| DOM7h-27 | 4G VH | 100 μg/ml HSA | 100 μg/ml HSA | HSA |
| | | tube | tube | |

dAbs that bound serum albumin on a BIACORE chip (Biacore AB) were then further analysed to obtain information on affinity. The analysis was performed using a CM5 chip (carboxymethylated dextran matrix) that was coated with serum albumin. Flow cell 1 was an uncoated, blocked negative control, flow cell 2 was coated with HSA, flow cell 3 was coated with RSA and flow cell 4 was coated with MSA. The serum albumins were immobilised in acetate buffer pH 5.5 using the BIACORE coating wizard which was programmed to aim for 500 resonance units (RUs) of coated material. Each dAb of interest was expressed in the periplasm of E. coli on a 200 mL-500 mL scale and purified from the supernatant using batch absorption to protein A-streamline affinity resin (Amersham, UK) for the V_{H}_{^{S}} and to protein L-agarose affinity resin (Affitech, Norway) for the V_{κ}s followed by elution with glycine at pH 2.2 and buffer exchange to PBS. A range of concentrations of dAb were prepared (in the range 5 nM to 5 μM) by dilution into BIACORE HBS-EP buffer and flowed across the BIACORE chip.

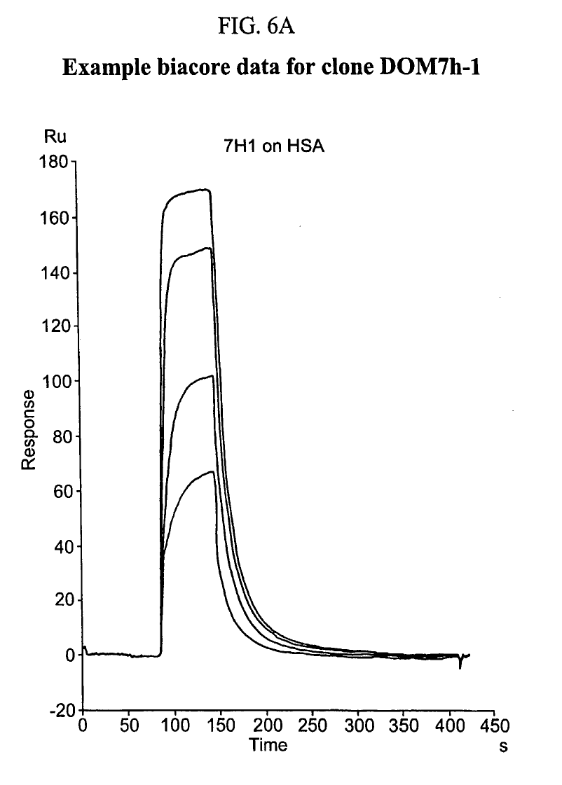

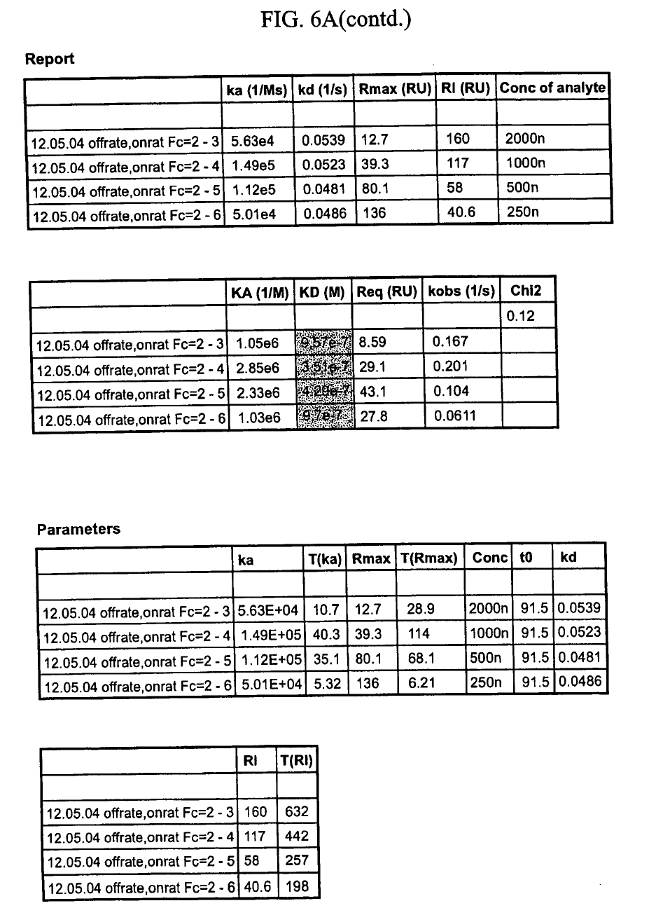

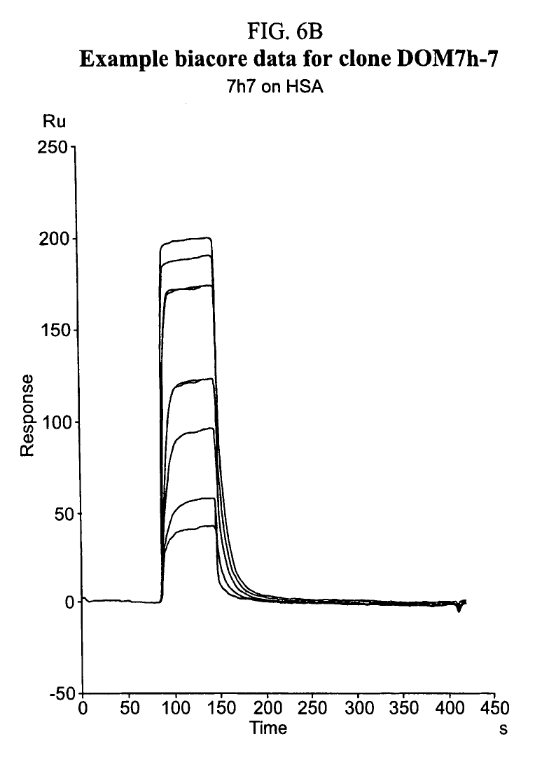

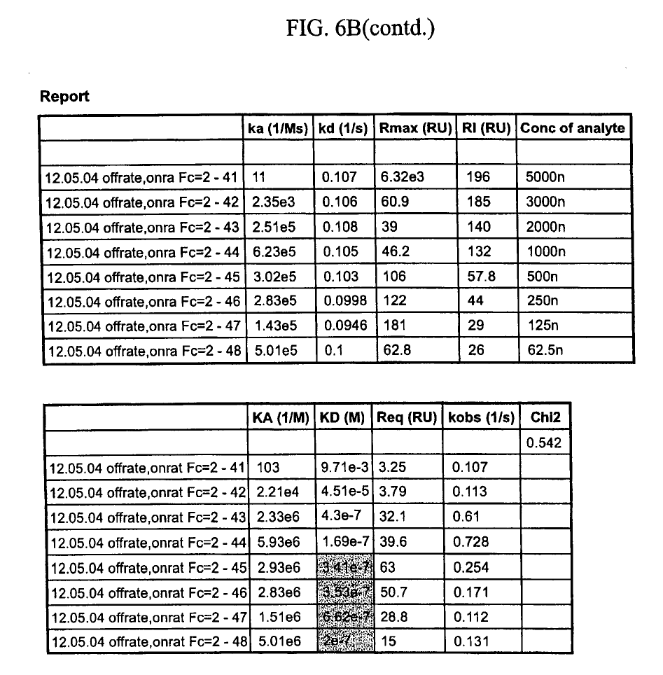

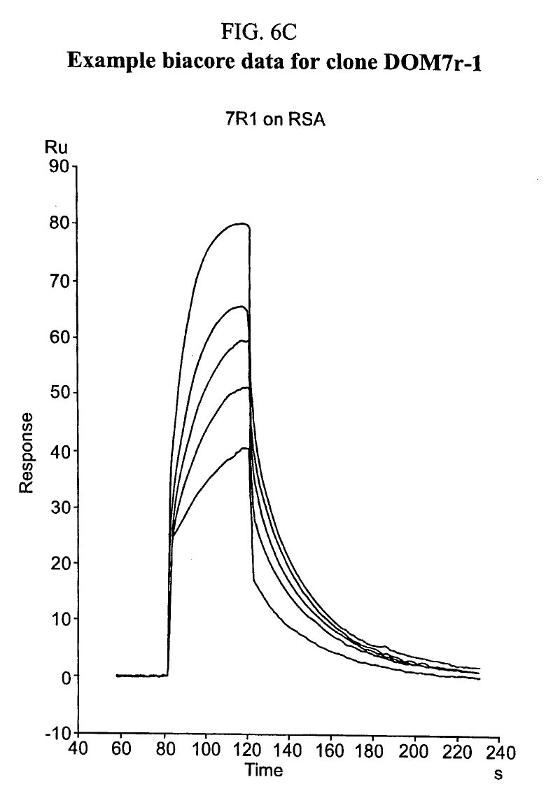

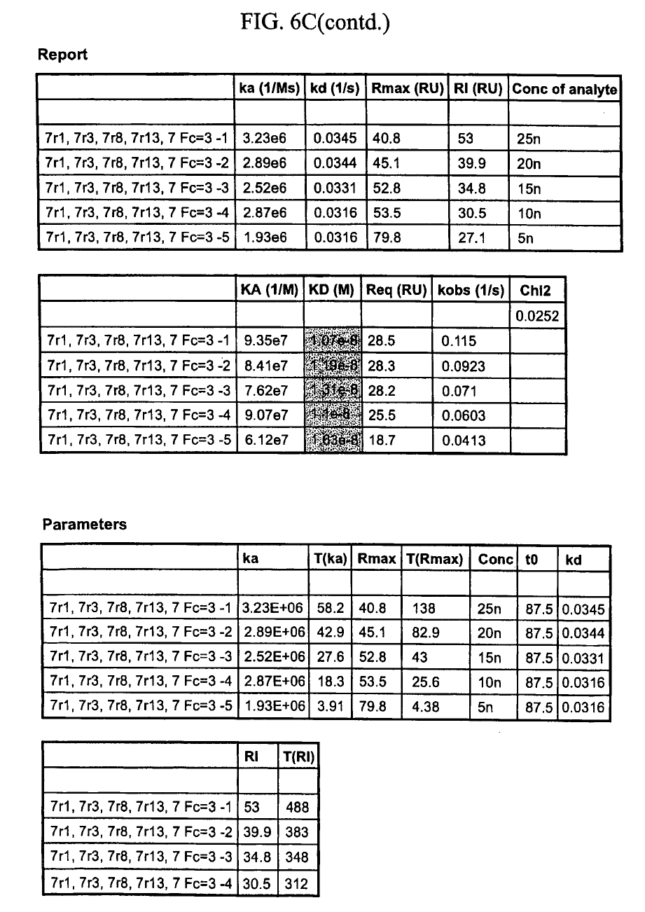

Affinity (KD) was calculated from the BIACORE traces by fitting onrate and offrate curves to traces generated by concentrations of dAb in the region of the KD. dAbs with a range of different affinities to serum albumin were identified. Included in the range 10-100 nM, were the affinities of DOM7h-8 for HSA, DOM7h-2 for HSA and DOM7r-1 for RSA. Included in the range 100 nM to 500 nM were the affinities of DOM7h-7 for HSA, DOM7h-8 for RSA and DOM7h-26 for HSA. Included in the range 500 nM to 5 μM were the affinities of DOM7h-23 for HSA and DOM7h-1 for HSA. Example traces are included in FIGS. 6A-6C.

### Example 2

### Formatting Anti-Serum Albumin Antibodies as a Fusion with IL-1 Receptor Antagonist (IL-1ra)

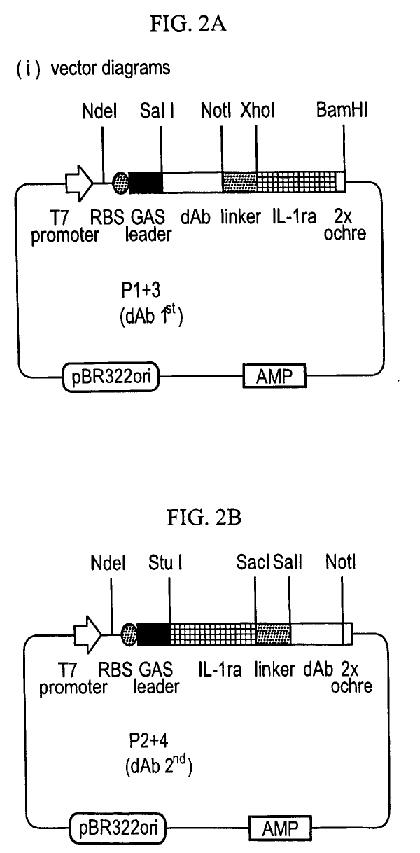

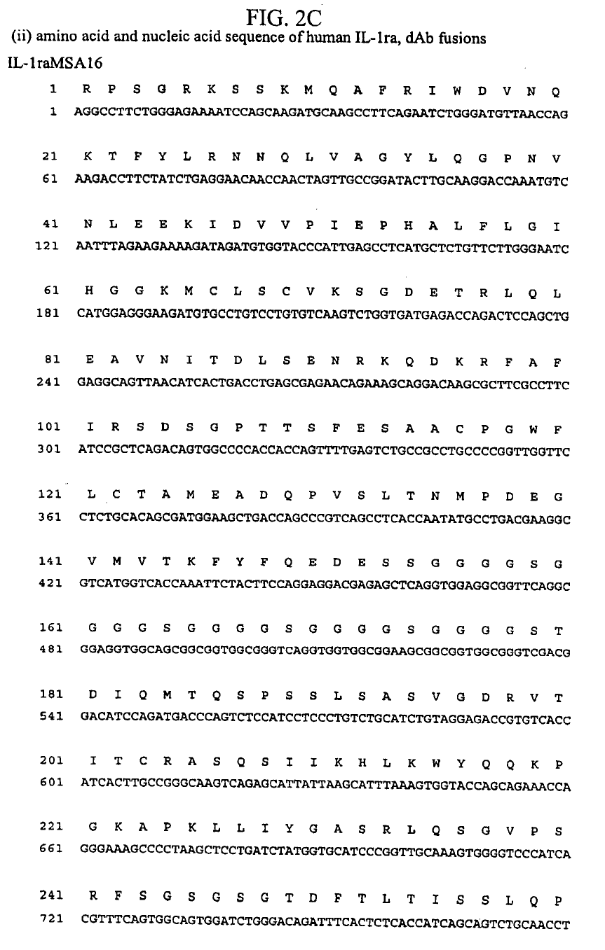

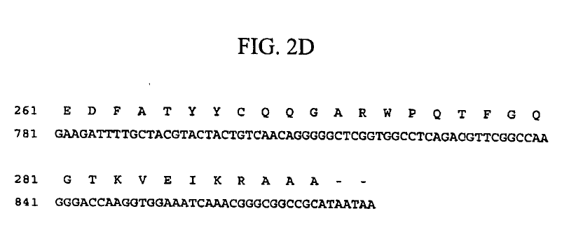

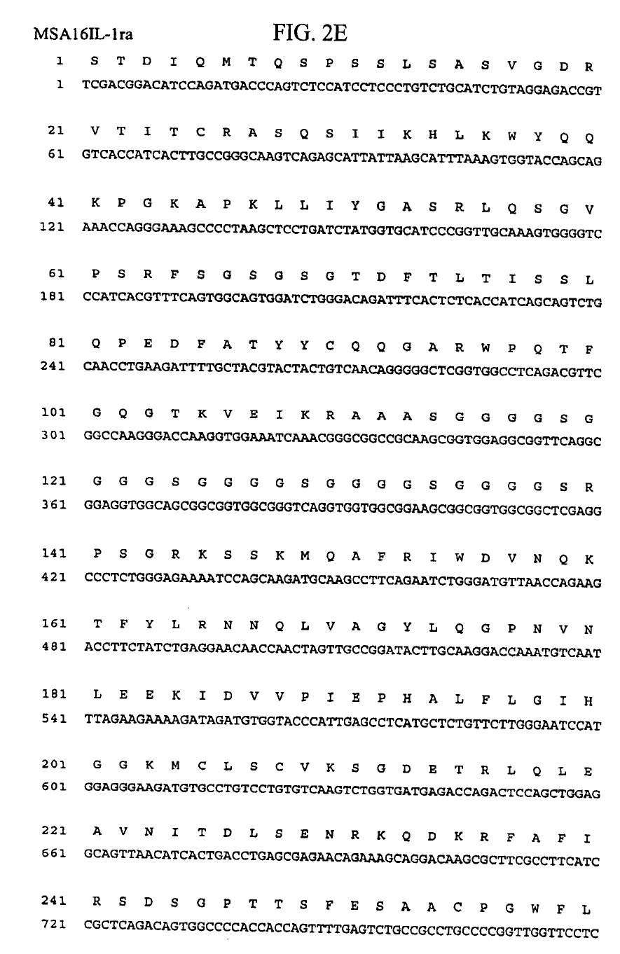

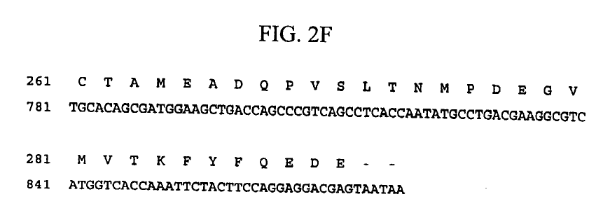

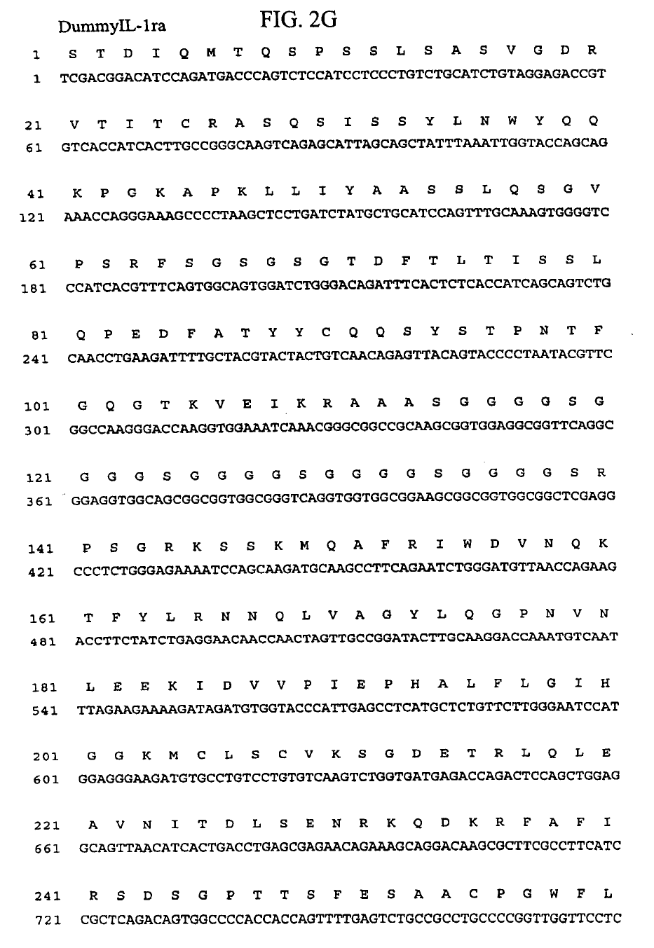

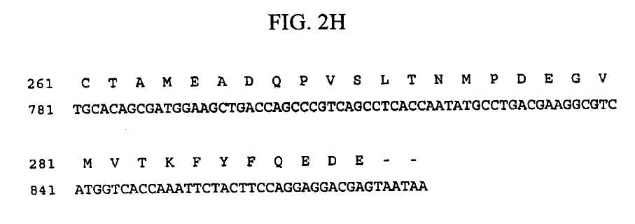

This example describes a method for making a fusion protein comprising IL-1ra and a dAb that binds to serum albumin. Two fusions were made, one with the dAb N-terminal of the IL-1ra (MSA16IL-1ra) and one with the dAb C-terminal of the IL-1ra (IL1-raMSA 16). The sequences of the fusions and the vector are shown in FIGS. 2C and 2D. A control fusion that did not bind MSA was also produced, and its sequence is shown in FIG. 2E.

KINERET (anakinra, Amgen Inc) has a short half-life of 4-6 hours, and the recommended dosing regime calls for daily injections. This regime lead to injection site reaction in 14-28 days in 71% of cases. Therefore a form of human IL-1ra that has a longer serum half-life would be beneficially and could increase efficacy and reduce dosing frequency. These are both desirable properties for a pharmaceutical.

### Cloning

Briefly, two multiple cloning sites (MCSs) were designed as detailed below and inserted into an expression vector with a T7 promotor. The restriction sites were designed for the insertion of IL1-ra, dAb, GAS leader and linker. One (MCS 1+3) encodes a protein with the dAb N terminal of the IL-1ra and the other (MCS 2+ 4) encode a protein with the dAb C terminal of the IL-1ra.

Cloning site 1+3 for dAbIL1-ra fusion
NdeI, stuffer, Sail, NotI, stuffer, XhoI, BamHI

(SEQ ID NO:35)

gcgcatatgttagtgcgtcgacgtcaaaaggccatagcgggcggccgctg
caggtctcgagtgcgatggatcc

Cloning site 2+4 for IL1-radAb fusion
NdeI, stuffer, StUI, SacI, stuffer, Sail, NotI, TAA TAA BamHI

(SEQ ID NO:36)

gcgcatatgttaagcgaggccttctggagagagctcaggagtgtcgacgg
acatccagatgacccaggcggccgctaataaggatccaatgc

The GAS leader was then inserted into each vector by digesting the MCS using the appropriate restriction enzymes and ligating annealed primers coding for the leader. Next, linker DNA coding for the linker was inserted in a similar manner. DNA coding for IL-1ra was obtained by PCR (using primers designed to add the required restriction sites) from a cDNA clone and inserted into a TOPO cloning vector. After confirming the correct sequence by nucleic acid sequencing, DNA coding for IL-1ra was excised from the TOPO vector and ligated into the vectors containing leader and linker. Lastly, DNA coding for the dAb was excised from the dAb expression vector and inserted into the vectors by SalI/NotI digest of insert (purified by gel purification) and vector.

### Expression and Purification

MSA16IL-1ra, IL1-raMSA16 and dummyIL-1ra were expressed in the periplasm of E. coli and purified from the supernatant using batch absorption to protein L-agarose affinity resin (Affitech, Norway) followed by elution with glycine at pH 2.2. The purified dAbs were then analysed by SDS-PAGE gel electrophoresis followed by coomassie staining. For one of the proteins (IL-1raMSA 16), > 90% of the protein was of the expected size and therefore was analysed for activity without further purification. The other proteins (MSA16IL-1ra and dummy IL-1ra) were contaminated by a smaller band and were therefore further purified by FPLC ion exchange chromatography on the RESOURSEQ ion exchange column at pH 9. Protein was eluted using a linear salt gradient form 0-500 mM NaCl. After analysis by SDS-PAGE gel electrophoresis, fractions containing a protein of the expected size were combined yielding a combined fraction of >90% purity. This protein was used for further analysis

### Example 3

### Determination of Activity of dAb IL1-ra Fusion In Vitro MRC-5 IL-8 Assay

MSA16IL-1ra fusions were tested for the ability to neutralize the induction of IL-8 secretion by IL-1 in MRC-5 cells (ATCC Accession No. CCL-171; American Type Culture Collection, Manassas, Va.). The method is adapted from Akeson, L. et al (1996) Journal of Biological Chemistry 271, 30517-30523, which describes the induction of IL-8 by IL-1 in HUVEC, MRC-5 cells were used instead of the HUVEC cell line. Briefly, MRC-5 cells plated in microtitre plates were incubated overnight with dAbIL-1ra fusion proteins or IL-1ra control, and IL-1 (100 pg/mL). Post incubation the supernatant was aspirated off the cells and IL-8 concentration measured via a sandwich ELISA (R&D Systems).

The activity of IL-1ra in the fusion proteins led to a reduction in IL-8 secretion. The reduction of IL-8 secretion resulting from activity of the MSA16IL1-ra fusion and from activity of the IL-1raMSA16 fusion was compared to the reduction seen with the IL-1ra control (recombinant human IL-1ra, R&D systems). The neutralizing dose 50 (ND50) of each of the tested proteins was determined and is presented in Table 2.

TABLE 2

| Protein | ND₅₀ | |
|---|---|---|
| IL-1ra | 0.5 | nM |
| MSA16IL-1ra | 2 | nM |
| IL-1raMSA16 | 8 | nM |

The results demonstrate that IL-1ra remained active as part of a fusion construct with an anti-serum albumin dAb. The MSA16IL-1ra protein was further studied to assess its pharmacokinetics (PK study).

Serum Albumin, anti IL-1ra sandwich ELISA

Three dAb/IL-1ra fusions were tested for the ability to bind serum albumin and simultaneously be detected by a monoclonal anti-IL1ra antibody. The fusions tested were MSA16IL-1ra, IL-1raMSA16 and dummyIL-1ra. Briefly, ELISA plate was coated overnight with mouse serum albumin at 10 μg/ml, washed 5× with 0.05% Tween PBS and then blocked for 1 hour with 4% Marvel PBS. After

blocking, the plate was washed 5× with 0.05% Tween PBS and then incubated for 1 hour with each dAb, IL-1ra fusion diluted in 4% MPBS. Each fusion was incubated at 1 μM concentration and at 7 sequential 4-fold dilutions (ie down to 60 pM). After the incubation, plates were washed 5× with 0.05% Tween PBS and then incubated for 1 hour with the manufacturers recommended dilution of a rabbit polyclonal antibody (ab-2573) to human IL-1 receptor antagonist (Abeam, UK) diluted in 4% MPBS. After this incubation, plates were washed 5× with 0.05% Tween PBS and then incubated for 1 h with a 1/2000 dilution of secondary antibody (anti-rabbit IgG-HRP) diluted in 4% MPBS. Following incubation with the secondary antibody, plates were washed 3× with 0.05% Tween PBS and 2× with PBS and then developed with 50 μl per well of TMB microwell peroxidase substrate (KPL, MA) and the reaction stopped with 50 μl per well of HCL. Absorbtion was read at 450 nM.

Both the MSA16IL-1ra and IL-1raMSA16 proteins were detected at more than 2× background level at 1 μM concentration in the sandwich ELISA. The MSA16IL-1ra protein was detected at 2× background or higher at dilutions down to 3.9 nM, whereas the IL-1raMSA16 protein was detected at 2× background only down to 500 nM. Binding of the MSA16IL-1ra fusion to serum albumin was shown to be specific for serum albumin as the control construct (dummyIL-1ra) did not bind serum albumin.

### Example 4

### Determination of Serum Half-Life of Drug Fusions in Mouse PK Studies

A. Determination of the Serum Half-Life in Mouse of a MSA Binding dAb/HA Epitope Tag Fusion Protein.

The MSA binding dAb/HA epitope tag fusion protein was expressed in the periplasm of E. coli and purified using batch absorption to protein L-agarose affinity resin (Affitech, Norway) followed by elution with glycine at pH 2.2. Serum half-life of the fusion protein was determined in mouse following a single intravenous (i.v.) injection at approx 1.5 mg/kg into CD1 strain male animals. Analysis of serum levels was by ELISA using goat anti-HA (Abeam, UK) capture and protein L-HRP (Invitrogen, USA) detection which was blocked with 4% Marvel. Washing was with 0.05% Tween-20, PBS. Standard curves of known concentrations of MSA binding dAb/HA fusion were set up in the presence of 1× mouse serum to ensure comparability with the test samples. Modelling with a 1 compartment model (WinNonlin Software, Pharsight Corp., USA) showed the MSA binding dAb/HA epitope tag fusion protein had a terminal phase 11/2 of 29.1 hours and an area under the curve of 559 hr·μg/ml. This demonstrates a large improvement over the predicted half-life for a HA epitope tag peptide alone which could be a short as only several minutes.

The results of this study using the HA epitope tag as a drug model, demonstrate that the in vivo serum half-life of a drug can be extended when the drug
is prepared as a drug fusion or drug conjugate with an antigen-binding fragment of (e.g., dAb) of an antibody that binds serum albumin.

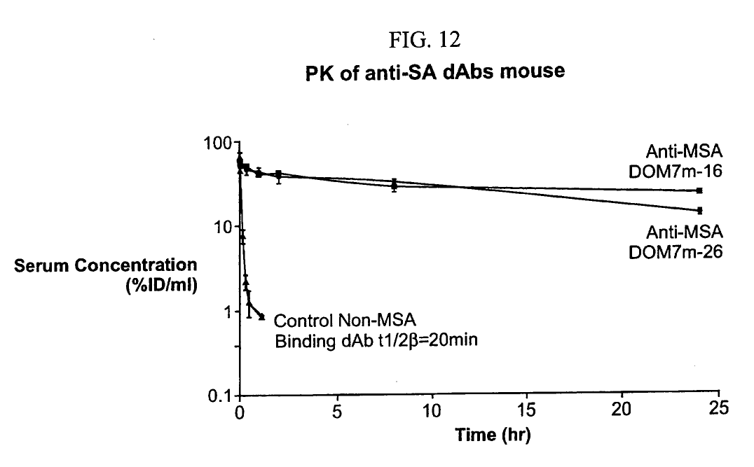

The in vivo half-life in mice of the anti-MSA dAbs DOM7m-16 and DOM7m-26, and a control dAb that does not bind MSA were also assessed. Again, DOM7m-16, DOM7m-26 and the control dAb contained an HA epitope tag, which serves as a model for a drug (e.g., a protein, polypeptide or peptide drug). In this study, the control dAb, that does not bind MSA, had an in vivo half-life of 20 minutes, whereas the in vivo half-lives of DOM7m-16 and DOM7m-26 were significantly extended. (FIG. 12) DOM7m-16 was found to have an in vivo half-life in mice of 29.5 hours in further studies.

In another study, the in vivo half-life (t½ β) of DOM7h-8 which contained an HA epitope tag was evaluated in mice. Modelling with a 2 compartment model (WinNonlin Software, Pharsight Corp., USA) showed that DOM7h-8 had a t½β of 29.1 hours.

The results of each of these study using the HA epitope tag as a model for a drug (e.g., a protein, polypeptide or peptide drug), demonstrate that the in vivo serum half-life of a drug can be dramatically extended when the drug is prepared as a drug fusion or drug conjugate with an antigen-binding fragment of (e.g., dAb) of an antibody that binds serum albumin.

B. Determination of the Serum Half-Life in Mouse of MSA Binding Dab/IL-1ra Fusion Protein.

The MSA binding dAb/IL-1ra fusion protein (MSA16IL-1ra) was expressed in the periplasm of E. coli and purified using batch absorption to protein L-agarose affinity resin (Affitech, Norway) followed by elution with glycine at pH 2.2. Serum half-life of the MSA16IL-1ra (DOM7m-16/IL-1ra), an IL-1ra fusion with a dAb that does not bind MSA (Dummy dAb/IL-1ra), and an anti-MSA dAb fused to the HA epitope tag (DOM7m-16 HA tag) was determined in mice following a single i.v. injection at approximately 1.5 mg/kg into CD1 strain male animals.

Analysis of serum levels was by II-1ra sandwich ELISA (R&D Systems, USA). Standard curves of known concentrations of dAb/IL-1ra fusion were set up in the presence of 1× mouse serum to ensure comparability with the test samples. Modelling was performed using the WinNonlin pharmacokinetics software (Pharsight Corp., USA).

It was expected that the IL-1ra fusion with the anti-MSA dAb would increase the serum half-life considerably when compared with the control which was a fusion of a non-MSA binding dAb with IL-1ra. The control non-MSA binding dAb/IL-1ra fusion was predicted to have a short serum half-life.

The results of the study are presented in Table 3, and show that the IL-1ra fusion with anti-MSA dAb (DOM7m-16/IL-1ra had a serum half-life that was about 10 times longer than the IL-1ra fusion with a dAb that does not bind MSA (Dummy dAb/IL-1ra). The results also revealed that there was a > 200 fold improvement (increase) in the area under the concentration time curve for DOM7m-16/IL-1ra (AUC: 267 hr·μg/ml) as compared to dummy/IL-1ra (AUC: 1.5 hr·μg/ml)

TABLE 3

| Agent | Serum Half-life | |
|---|---|---|
| DOM7m-16/IL-1ra | 4.3 | hours |
| dummy/IL-1ra | 0.4 | hours |
| DOM7m-16 HA tag | 29 | hours |

The results of these studies demonstrate that the in vivo serum half-life and AUC of a drug can be significantly extended when the drug is prepared as a drug fusion or drug conjugate with an antigen-binding fragment of (e.g., dAb) of an antibody that binds serum albumin.

### Example 5

### Determination of the Serum Half-Life in Rats of RSA Binding dAb/HA Epitope Tag Fusion Proteins

Anti-rat serum albumin dAbs were expressed with C-terminal HA tags in the periplasm of E. coli and purified using batch absorption to protein L-agarose affinity resin (Affitech, Norway) for Vk dAbs and batch absorption to protein A affinity resin for VH dAbs, followed by elution with glycine at pH 2.2. In order to determine serum half-life, groups of 4 rats were given a single i.v. injection at 1.5 mg/Kg of DOM7r-27, DOM7r-31, DOM7r-16, DOM7r-3, DOM7h-8 or a control dAb (HEL4) that binds an irrelevant antigen. Serum samples were obtained by serial bleeds from a tail vein over a 7 day period and analyzed by sandwich ELISA using goat anti-HA (Abeam, Cambridge UK) coated on an ELISA plate, followed by detection with protein A-HRP (for the V_{H} dAbs) or protein L-HRP (for V_{κ} dAbs). Standard curves of known concentrations of dAb were set up in the presence of 1× rat serum to ensure comparability with the test samples. Modelling with a 2 compartment model (using WinNonlin pharmacokinetics software (Pharsight Corp., USA)) was used to calculate 11/2/3 and area under the curve (AUC) (Table 4). The t½β for HEL4 control in rats is up to 30 minutes, and based on the data obtain the AUC for DOM7h-8 is expected to be between about 150 hr·μg/mL and about 2500 hr·μg/mL.

TABLE 4

| | | Affintity (KD) | | | |
|---|---|---|---|---|---|
| | | for rat serum | | | AUC |
| Agent | Scaffold | albumin | | t½β | (hr · μg/mL) |
| DOM7r-3 | V_{κ} | 12 | nM | 13.7 hours | 224 |
| DOM7r-16 | Vκ | 1 | μM | 34.4 hours | 170 |
| DOM7r-27 | V_{H} | 250 | nM | 14.8 hours | 78.9 |
| DOM7r-31 | V_{H} | 5 | μM | 5.96 hours | 71.2 |

The results of this rat study using the HA epitope tag as a model for a drug (e.g., a protein, polypeptide or peptide drug), demonstrate that the in vivo serum half-life of a drug can be dramatically extended when the drug is prepared as a drug fusion or drug conjugate with an antigen-binding fragment of (e.g., dAb) of an antibody that binds serum albumin.

### Prediction of Half-Life in Humans.

The in vivo half-life of a dAb, drug fusion or drug conjugate in humans can be estimated from half-life data obtained in animals using allometric scaling. The log of the in vivo half-lives determined in 3 animals is plotted against the log of the weight of the animal. A line is drawn through the plotted points and the slope and y-intercept of the line are used to calculate the in vivo half-life in humans using the formula log Y= log(a)+b log(W), in which Y is the in vivo half-life in humans, log(a) is the y-intercept, b is the slope, and W is the weight of a human. The line can be produced using in vivo half-life data obtain in animals that weigh about 35 grams (e.g., mice), about 260 grams (e.g., rats) and about 2,710 grams. For this calculation, the weight of a human can be considered to be 70,000 grams. Based on half-life values obtained in mice and rats, dAbs that bind human serum albumin, such as DOM7h-8, are expected to have t½β of about 5.5 hours to about 40 hours and AUC of about 150 hr·μg/mL to about 2500 hr·μg/mL, in humans.

### Example 6

### Efficacy of Anti-SA dAb/IL-1ra Drug Fusion in Mouse Collagen Induced Arthritis Model of Rheumatoid Arthritis

Efficacy of the fusion DOM7m-16/IL-1ra and efficacy of IL-1ra in a recognized mouse model of rheumatoid arthritis (type II collagen induced arthritis (CIA) in DBA/1 mice) was assessed. Throughout the study, mice were maintained in a test facility in standard type 2 cages that were housed in a HEPA-filtered Scantainer at 20-24° C. with a 12-hours light, 12-hours dark cycle. Food (Harlan-Teklad universal diet 2016) and UV sterilized water were provided ad libitum. The mice were imported to the test facility at least 7 days before the start the study to assure proper acclimatization.

DBA/1 mice at 7-8 weeks of age (obtained from Taconic M and B, Domholtveg, Denmark) were injected once with an emulsion of Arthrogen-CIA adjuvant and Arthrogen-CIA collagen (both MD biosciences) emulsified at a 1:1 ratio until the emulsion was stable. The emulsion was considered to be stable when a drop of the emulsion added to a beaker of water formed a solid clump. The mice were then injected with the emulsion.

Twenty-one days after the emulsion was injected, the 20 animals with the most advanced arthritic disease were eliminated from the study, and the remaining mice were divided into groups of 10 animals (each group contained 5 males and 5 females). The mice were treated as shown in Table 5, and all treatments were delivered at a concentration calculated so that 10 ml/Kg were administered.

TABLE 5

| Group | Treatment |
|---|---|
| 1 | IL-1ra, 1 mg/Kg (intrapertoneal (ip.) bolus) |
| 2 | IL-1ra, 10 mg/Kg (ip. bolus) |
| 3 | DOM7m-16/IL-1ra, 1 mg/Kg (ip. bolus) |
| 4 | DOM7m-16/IL-1ra, 10 mg/Kg (ip. bolus) |
| 5 | ENBREL ® (entarecept; Immunex Corporation), 5 mg/Kg |
| | (ip. bolus) |
| 6 | saline (negative control), 10 ml/Kg (ip. bolus) |
| 7 | Dexamethasone (positive control), 0.4 mg/Kg |
| | (subcutaneous injection) |

Clinical scores for the severity of arthritis were recorded 3 times a week from day 21 to day 49. Mice were euthanized at day 49. Individual mice were euthanized earlier if they presented an arthritic score of 12 or more, or had serious problems moving.

For clinical scoring, each limb was scored according to the criteria below and the scores for all four limbs were added to produce the total score for the mouse. This method resulted is a score of 0 to 16 for each mouse. Scoring criteria were: 0=normal; 1=mild but definite redness and swelling of the ankle or wrist, or apparent redness and swelling limited to individual digits, regardless of the number of affected digits; 2=moderate redness and swelling of ankle and wrist; 3=severe redness and swelling of the entire paw including digits; 4=maximally inflamed limb with involvement of multiple joints.

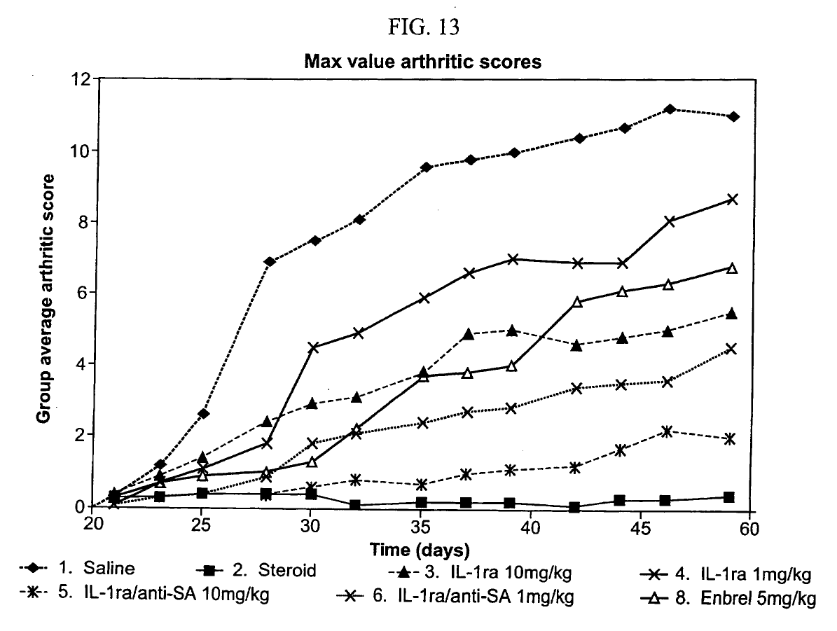

Group average arthritic scores were calculated for each treatment group on every treatment day using clinical scores from individual mice. Any animals that had been removed from the study for ethical reasons were allocated the maximum score of 16. The group average arthritic scores were plotted against time (FIG. 13).

Statistical analysis of the group average arthritic scores on day 49 were performed using the Wilcoxon test. This statistical analysis revealed that the two groups treated with DOM7m-16/IL-1ra (at 1 mg/Kg or 10 mg/Kg (Groups 3 and 4)) had significantly improved arthritic scores at day 49 (at the P<1% and P<0.05% significance levels respectively) when compared to the saline control group (Group 6). In contrast, treatment with IL-1ra at 1 mg/Kg (Group 1) did not result in statistically significant improvement in the arthritic score at day 49, while treatment with IL-1ra at 10 mg/Kg (Group 2) resulted in a significant improvement at the P <5% significance level. Treatment with ENBREL® (entarecept; Immunex Corporation) (Group 5) resulted in significant improvement in the arthritic score at day 49 at the P<10% significance level.

Treatment with DOM7m-16/IL-1ra at the 10 mg/Kg dose (Group 4), was effective at improving the arthritic score at day 49 (significant at the P<0.5% level) when compared to standard treatment with ENBREL® (entarecept; Immunex Corporation) at 5 mg/Kg (Group 5). In addition, treatment with DOM7m-16/IL-1ra
at the lower 1 mg/Kg dose (Group 3), was more efficacious at improving the arthritic score at day 49 than treatment with IL-1ra alone at the same dosage (Group 1) (significant at the P<10% level).

The results of the study show that at certain doses DOM7m-16/IL-1ra was more effective than IL-1ra or ENBREL® (entarecept; Immunex Corporation) in this study. The response to IL-1ra was dose dependent, as expected, and the response to DOM7m-16/IL-1ra was also dose dependent. The average scores for treatment with DOM7m-16/IL-1ra at 1 mg/Kg were consistently lower than the average scores obtained by treatment with IL-1ra at 10 mg/kg. These plotted results (FIG. 13) indicate that treatment with DOM7m-16/IL-1ra was about 10 times more effective than IL-1ra in this study.

This superior efficacy of DOM7m-16/IL-1ra was observed even though the DOM7-16/IL-1ra fusion protein contains about half the number of IL-1 receptor binding epitopes as IL-1ra on a weight basis (e.g., 1 mg of DOM7m-16/IL-1ra (MW .31.2 kD) contains about half the number of IL-1 receptor binding epitopes as 1 mg of IL-1ra (MW .17.1 kD).

The results of this study demonstrate that a dAb that binds serum albumin can be linked to IL-1ra (a clinically proven therapy for RA) and that the resulting drug fusion has both long serum half-life properties (conferred by the dAb) and IL-1 receptor binding properties (conferred by the IL-1ra). Due to the serum residence time of the drug fusion, the dose of DOM7-16/IL-1ra that was effective for treating CIA was dramatically reduced relative to IL-1ra.

The results of this study demonstrate that in addition to the benefits of extended half-life and increased AUC, drugs prepared as drug fusions or drug conjugates with an antigen-binding fragment of (e.g., dAb) of an antibody that binds serum albumin are highly effective therapeutic agents that provide advantages over drug alone. For example, as demonstrated in the mouse CIA model, a lower dose of drug fusion was effective and inhibited the joint inflammation and joint damage caused by IL-1 over a longer period of time in comparison to IL-1ra alone, and provided greater protection against disease progression.

### Example 7

### Anti-Sa Dab/Saporin Noncovalent Drug Conjugate

The ribosome-inactivating protein Saporin (an anti-cancer drug) is highly stable to denaturants and proteases and has been used as a targeted toxin to T lymphocytes. A non-covalent drug conjugate was prepared by coupling Saporin to DOM7h-8 via a biotin-streptavidin link. Results obtained with this non-covalent drug conjugate demonstrates that the DOM7h-8 retains its serum albumin binding characteristics when coupled to a drug.

A variant DOM7h-8 referred to as DOM7h-8cys, in which the C-terminal arginine at position 108 (amino acid 108 of SEQ ID NO:24) was replaced with a cysteine residue was prepared by expression of a recombinant nucleic acid in HB2151 cells. The cells were grown and induced at 30° C. in overnight expression autoinduction TB readymix (Merck K Ga, Germany) for 72 hours before recovery of the supernatant by centrifugation. DOM7h-8cys was purified from the supernatant using affinity capture on protein L-agarose. The resin was then washed with 10 column volumes of 2×PBS and DOM7h-8cys was eluted with 0.1 M glycine pH2. Eluted DOM7h-8cys was neutralized with 0.2× volume of Tris pH8 and concentrated to 1 mg/ml (using a CENTRICON 20 ml concentrator (Millipore Corp., MA).

Concentrated DOM7h-8cys was buffer exchanged to PBS using a NAP5 desalting column (GE Healthcare/Amersham Biosciences, NJ) and concentration determined. The dAb was then biotinylated (via primary amines) using EZ-LINK sulfo-NHS-LC-biotin (Pierce Biotechnology Inc., IL). The biotinylated dAb was mixed with streptavidin-saporin (Advanced Targeting Systems, San Diego) in a 1:1 molar ratio.

In order to confirm that the dAb/saporin complex was formed, a sandwich ELISA was used to detect intact complexes. Human serum albumin (HSA) was coated onto half of the wells of an ELISA plate (Nunc, N.Y.) overnight at 10 μg/ml in a volume of 100 μl per well. After overnight incubation, the plate was washed 3 times with PBS, 0.05% Tween and then the whole plate was blocked for 2 hours with 2% PBS. After blocking, the plate was washed 3 times with PBS, 0.05% Tween and then incubated for 1 hour with DOM7h-8/saporin non-covalent conjugate diluted to 0.5 μM in 2% Tween PBS. As controls on the same ELISA plate, uncoupled saporin at 0.5 μM and uncoupled DOM7h8 at 0.5 μM were incubated in 2% Tween PBS. Additional controls were the same three diluted proteins incubated on wells of the ELISA plate not coated with HAS and blocked with 2% Tween. After the incubation, the plate was washed 3 times with PBS, 0.05% Tween and then incubated for 1 hour with 1/2000 dilution of goat anti-saporin polyclonal antibody (Advanced Therapeutic Systems) diluted in 2% Tween PBS. After the incubation, the plate was washed 3 times with PBS, 0.05% Tween and then incubated for 1 hour with the secondary detection antibody (of 1/2000 anti-goat Ig HRP conjugate). After the incubation, the plate was washed 3 times with PBS, 0.05% Tween and once with PBS and tapped dry on paper. The ELISA was developed with 100 μl 3,3′,5,5′-tetramethylbenzidine as substrate and the reaction stopped with 50 μl 1M hydrochloric acid. The presence of non-covalent conjugates of DOM7h-8 and saporin was confirmed by comparing the OD600 of the conjugate with that of either of the unconjugated parts.

TABLE 6

| | DOM7h-8/ | DOM7h-8 | Saporin |
|---|---|---|---|
| | Saporin | alone | alone |
| OD600 | 0.311 | 0.060 | 0.079 |
| (plate coated with HAS) | | | |
| OD600 | 0.078 | 0.068 | 0.075 |
| (plate blocked with 2% | | | |
| Tween PBS) | | | |

The results of this study demonstrate that a drug can be conjugated to an antigen-binding fragment of an antibody that binds serum albumin, and that the conjugated antigen-binding fragment retains serum albumin-binding activity. In addition, due to the stability and strength of the biotin-streptavidin interation, the results show that covalently bonded and noncovalently bonded conjugates can be prepared that retain the serum albumin-binding activity of the antigen-binding fragment of an antibody that binds serum albumin.

### Example 8

### Anti-SA dAb/Fluorescein Conjugate

Fluorescein isothiocyanate (FITC) can be cross linked with amino, sulfhydryl, imidazoyl, tyrosyl or carbonyl groups on a protein. It has a molecular weight of 389 Da which is comparable in size to many small molecule drugs. Results obtained with this conjugate demonstrate that the anti-SA dAb maintains its serum albumin binding characteristics when coupled to a small chemical entity, and indicate that small molecule drugs can be conjugated to anti-SA dAbs.

Concentrated DOM7h-8cys was prepared as described in Example 7. The concentrated dAb was buffer exchanged to 50 mM Borate pH 8 (coupling buffer) using a NAP5 desalting column (GE Healthcare/Amersham Biosciences, NJ) and then concentrated to 2.3 mg/ml using a 2 ml CENTRICON concentrator (Millipore
Corp., MA). The FITC (Pierce Biotechnology Inc.) was diluted to 10 mg/ml in dimethyl formamide (DMF) according to the manufacturer's instructions and then mixed with the dAb in coupling buffer at a molar ratio of 24:1 FITC:dAb. The reaction was allowed to proceed for 30 minutes. At this point, excess unreacted FITC was removed from the reaction using a PD10 desalting column (GE Healthcare/Amersham Biosciences, NJ) that was pre-equilibrated with PBS, and the DOM7h-8cys/FITC conjugate was eluted with PBS.

In order to confirm that the FITC/dAb coupling reaction was successful, a sandwich ELISA was used to detect coupled dAb. Human serum albumin (HSA) was coated onto half of the wells of an ELISA plate (Nunc, N.Y.) overnight at 10 μg/ml in a volume of 100 μl per well. After overnight incubation, the whole plate was washed 3 times with PBS, 0.05% Tween and then all the wells were blocked for 2 hours with 2% Tween PBS. After blocking, the plate was washed 3 times with PBS, 0.05% Tween and then incubated for 1 hour with DOM7h-8cys/FITC diluted to 1 μM in 2% Tween PBS. As controls on the same ELISA plate, a control FITC coupled antibody at 1 μM and uncoupled DOM7h-8 at 1 μM were incubated in 2% Tween PBS. Additional controls were the same three diluted proteins incubated on wells of the ELISA plate not coated with HSA and blocked with 2% Tween. After the incubation, the plate was washed 3 times with PBS, 0.05% Tween and then incubated for 1 hour with 1/500 dilution of rat anti FITC antibody (Serotec) diluted in 2% Tween PBS. After the incubation, the plate was washed 3 times with PBS, 0.05% Tween, and then incubated for 1 hour with the secondary detection antibody diluted in 2% Tween PBS (1/5000 anti-rat Ig HRP conjugate). After the incubation, the plats was washed 3 times with PBS, 0.05% Tween and once with PBS and tapped dry on paper. The ELISA was developed with 100 μl per well 3,3′,5,5′-tetramethylbenzidine as substrate and the reaction stopped with 50/al per well 1M hydrochloric acid. The presence of conjugates of DOM7h-8 and FITC was confirmed by comparing the OD600 of the conjugate with that of either of the unconjugated parts.

TABLE 7

| | | | FITC coupled |
|---|---|---|---|
| | DOM7h- | DOM7h-8 | antibody |
| | 8/FITC | alone | (negative control) |
| OD600 | 0.380 | 0.042 | 0.049 |
| (plate coated with HSA) | | | |
| OD600 | 0.041 | 0.041 | 0.045 |
| (plate blocked with 2% | | | |
| Tween PBS) | | | |

### Example 9

### Anti-SA dAb/Peptide Conjugates

Many peptides have therapeutic effects. Model peptides with an N- or C-terminal cysteine can be coupled to an anti-serum albumin dAb.

In this case, four different peptides will be used: peptide 1 YPYDVPDYAKKKKKKC (SEQ ID NO:64); peptide 2 CKKKKKKYPYDVPDYA (SEQ ID NO:65); peptide 3 HHHHHHKKKKKKC (SEQ ID NO:66) and peptide 4: CKKKSGOCHHHHHH (SEQ ID NO:67). Peptides 1 and 2 include the sequence of the hemagglutinin tag (HA tag) and peptides 3 and 4 include the sequence of the His tag. Concentrated DOM7h-8cys will be prepared as described in Example 7.

The concentrated dAb will be reduced with 5 mM dithiothreitol and then buffer exchanged to coupling buffer (20 mM BisTris pH 6.5, 5 mM EDTA, 10% glycerol) using a NAP5 desalting column (GE Healthcare/Amersham Biosciences, NJ). Cysteines will be blocked (to prevent the dAb dimerizing with itself) using a final concentration of 5 mM dithiodipyridine which will be added to the dAb solution form a stock of 100 mM dithiodipyridine in DMSO. The dAb and dithiodipyrdine will be left to couple for 20-30 minutes. Unreacted dithiodipyridine will then be removed using a PD10 desalting column and the dAb will be eluted in coupling buffer (20 mM BisTris pH 6.5, 5 mM EDTA, 10% glycerol). The resulting protein will then be frozen until required.

Peptides 1-4 will be individually dissolved in water at a concentration of 200 μM, will be reduced using 5 mM DTT and then will be desalted using a NAP5 desalting column (GE Healthcare/Amersham Biosciences, NJ). Each peptide will then be added to a solution of reduced and blocked dAb at a 20:1 ratio, for the peptide-dAb coupling to occur. In order to confirm success of the peptide, dAb coupling reactions, a sandwich ELISA will be used to detect anti-SA dAb/peptide conjugates.

Human serum albumin will be coated onto an ELISA plate (Nunc, N.Y.) overnight at 10 μg/ml in a volume of 100 μl per well. After overnight incubation, the plate will be washed 3 times with PBS, 0.05% Tween and then will be blocked for 2 hours with 4% Marvel PBS. After blocking, the plate will be washed 3 times with PBS, 0.05% Tween and then will be incubated for 1 hour with DOM7h-8/peptide conjugates diluted to 1 μM in 4% Marvel PBS. As controls on the same ELISA plate, uncoupled peptide at 20 μM and uncoupled DOM7h-8 at 1 μM will be incubated in 4% MPBS. After the incubation, the plate will be washed 3 times with PBS, 0.05% Tween and then will be incubated for 1 hour with 1/2000 dilution of goat anti-HA antibody (Abeam) for peptides 1 and 2, and a 1/2000 dilution of Ni NTA-HRP (for peptides 3 and 4) diluted in 4% Marvel PBS. After incubation, the plate will be washed 3 times with PBS, 0.05% Tween and the wells with the goat 1 anti HA antibody will be incubated for 1 h with secondary anti-goat HRP antibody diluted 1/2000 in 4% MPBS (other wells were blocked for 1 h). After the incubation, the plate will be washed 3 times with PBS, 0.05% Tween and once with PBS and will then be tapped dry on paper. The ELISA will be developed with 3,3′,5,5′-tetramethylbenzidine as substrate and the reaction will be stopped with 1M hydrochloric acid. The presence of conjugates of DOM7h-8/peptide conjugate will be confirmed by comparing the OD600 of the conjugate with that of either of the unconjugated parts.

### Example 10

This prophetic example describes suitable methods that will be used for the production, purification and characterization of protein fusions comprising a human PLAD domain and an immunoglobulin variable domain that binds serum albumin, Fusion proteins will be produced in which pre-ligand assembly domain of human TNFR1 (PLAD domain) is fused to the N-terminus of an immunoglobulin variable domain that binds human serum albumin (DOM7h-8) (yielding PLAD-DOM 7h-8) or in which the PLAD is fused to the C-terminus of the immunoglobulin variable domain that binds serum albumin (yielding DOM7h-8-PLAD). The amino acid sequence of PLAD is derived from a cDNA sequence isolated from a human library, and is amino acid residues 1-51 of SEQ ID NO:85. The amino acid sequence of DOM7h-8 is SEQ ID NO:24. These proteins will be expressed in three different expression organisms: Echerichia coli, Pichia pastoris and mammalian cells such as HEK293T cells, purified and tested in a range of in vitro assays and in vivo studies.

The following nucleotide sequence encodes amino acid residues 1-51 of SEQ ID NO:85:

(SEQ ID NO:98)

CTGGCCCTCACCTAGGGGACAGGGAGAAGAGAGATAGTGTGTGTCCCCAA
GGAAAATATATCCACCCTCAAAATAATTCGATTTGCTGTACCAAGTGCCA
CAAAGGAACCTACTTGTACAATGACTGTCCAGGCCCGGGGCAGGATACGG
ACTGCAGG

The following nucleotide sequence encodes DOM7h-8:

(SEQ ID NO:99)

GACATCCAGATGACCCAGTCTCCATCCTCCCTGTCTGCATCTGTAGGAGA
CCGTGTCACCATCACTTGCCGGGCAAGTCAGAGCATTAGCAGCTATTTAA
ATTGGTATCAGCAGAAACCAGGGAAAGCCCCTAAGCTCCTGATCTATCGG
AATTCCCCTTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTCAACAGACGTATAGGGTGCCTCCTACGTTCGGCCAA
GGGACCAAGGTGGAAATCAAACGG

### Fusion Gene Construction, Cloning and Expression

The fusion gene products will be produced by polymerase chain reaction (PCR) in which both genes are amplified in two separate reactions using a pair of primers that contain an overlapping sequence. The overlapping sequence will also be used to introduce a polypeptide linker sequence of varying length and compositions (e.g. a flexible six amino acid peptide such as Thr-Val-Ala-Ala-Pro-Ser (SEQ ID NO:100). The two PCR products formed in this way will be fused together by a process called SOE-PCR (‘splicing-by-overlap-extension PCR’) in which both templates will be mixed together (at 1:1 ratio) and submitted to several rounds of PCR amplification in the absence of primers. The newly formed fused PCR product will then be further amplified by PCR using a pair of external primers that encompass at least the whole fusion gene. Primers will be designed to introduce restriction sites at either end of the gene fusion product. The gene fusion product will be digested with restriction endonuclease(s) specific for the restriction sites, purified and subsequently ligated into the multiple cloning sites of suitable vectors for the expression system, in fusion with any required amino-terminal secretion and processing sequences in the vector, The primers that will to be used for each reaction to produce a fusion gene that encodes a fusion protein with an intervening DNA segment coding for 6 amino acid linker (ThrValAlaAlaProSer (SEQ ID NO:100) are given in Table 9. The sequences of the primers are given in Table 10.

The vectors that will be used are;

pUC119 for E. coli: The yeast glycolipid anchored surface protein secretion signal (GAS) will be cloned in-frame as a amino-terminus leader sequence to target expression of the fusion product to the E. coli periplasm (a suitable environment for oxidation of cysteines to form disulfide bonds). The leader sequence will be removed by the E. coli signal peptidase to leave the native amino terminus of the PLAD-DOM7h-8 or DOM7h-8-PLAD fusion product. Expression in this system will be driven by the P_{tac} promoter and induced by the addition of isopropyl-thio-beta-galactoside (IPTG) at 0.05 to 1 mM final concentration to exponentially growing cultures.

pDOM32 for expression in mammalian cells (such as HEK293T cells): The PLAD-DOM7h-8 (or the DOM7h-8-PLAD) will be cloned such that the fusion product is in frame with the V-J2-C secretion signal sequence. Expression is driven constitutively by the CMV promoter of pDOM32 in HEK-293 cells. On secretion, the signal peptide will be removed to yield an intact fusion protein with no additional amino-acids at the amino-terminus.

pET23 for E. coli: The PLAD-DOM7h-8 (or the DOM7h-8-PLAD) will be expressed as an insoluble protein in the E. coli cytoplasm without any leader upon IPTG induction. The proteins will have an additional amino-terminal methionine residue at the N-terminal end of the fusion product(s). Expression in this system will be induced by the addition of isopropyl-thio-beta-galactoside (IPTG) at 0.05 to 1 mM final concentration to exponentially growing cultures.

pPICZα for expression in Pichia pastoris: The PLAD-DOM7h-8 (or the DOM7h-8-PLAD) will be cloned in frame with the yeast alpha mating factor leader sequence to direct secretion to the culture supernatant. The leader sequence will be removed on secretion by the Kex2 and Ste13 proteases to leave a protein with no additional amino acids at the amino-terminus. Expression in this system will be induced by the addition of 100% methanol to the culture medium (0.5% to 2.5% final volume)

The recombinant fusion genes will to be cloned into the multiple cloning site of pUC119 using Sail and NotI, into pDOM32 using BamHI and HindIII, and into pPICZα using XhoI and NotI.

The plasmids containing insert will first be transformed into E. coli cells. The plasmids will then be removed and the genes of interest sequenced to confirm the presence of the correct gene sequences. Plasmids will then be prepared in large quantities and used to transform into the suitable cells for protein expression.

Suitable cells for expression using the pUC119 vector will be chosen form the following: TG1, TB1, HB2151, XL-1 Blue, DH5, UT5600, W3110, etc. Suitable cells for expression using the pDOM32 vector will be chosen form the following: HEK293T cells, NS1, COS, CHO, etc. Suitable cells for expression using the pET23 vector will be chosen form the following: BL21(DE3), BL21(DE3)pLysS, PL21(DE3)ₚLysE, BL21 Tuner, Origami, Rosetta, etc. Suitable cells for expression using the pPICZα vector will be chosen from the following: KM71H, X33.

With pUC119-, pDOM32- and pPICZα-based expression, the fusion product will be secreted in the culture supernatant. Therefore, following expression, the cultures will be spun down to pellet the cells. The supernatants will be recovered, filtered to remove remaining cells and directly processed for purification. With pET-23-based expression, the fusion product will accumulate into the periplasm as inclusion bodies. Inclusion bodies will be prepared according top methods well-known in the art involving a cell lysis step and several wash steps to clean the inclusion bodies. The inclusion bodies will be solubilized by addition of denaturants at high concentration (e.g., urea, guanidinium hydrochloride) and reducing agents (e.g., DTT, beta-mercapto ethanol, TCEP). Refolding of the fusion products will be performed according to methods well-known in the art, either by slow-dialysis in buffer with decreasing amounts of denaturants, or by rapid-dilution in refolding buffer. Additives such as L-arginine, glycerol, protease inhibitors such as PMSF and oxido-reduction agents such as GSH and GSSG will be added to the refolding buffer to improve the folding yield.

### Purification of Fusion Proteins

Fusion proteins will be affinity-purified on a Peptostreptococcal Protein L agarose column. This utilises the specific high affinity interaction between the immunoglobulin variable domain component of the PLAD-DOM7h-8 (or the DOM7h-8-PLAD) fusion protein with Protein L. Typically, the sample will be loaded on the protein L column at neutral pH. The column will be washed at neutral pH with high salt, the sample will be eluted by addition of a low pH buffer. The eluted sample will be collected and the pH neutralized. Any remaining contaminants will be removed by cation- or anion-exchange, size exclusion chromatography, hydrophobic interaction chromatography or another suitable method.

The identity of the purified fusion protein will be confirmed by amino-terminus sequencing, and MALDI-mass spectrometry analysis, such that the sequence and mass obtained match those predicted based on the DNA sequence.

### Activity of Fusion Proteins

The fusion products with any linker will then be assayed for biological activity.

PLAD activity: Human MRC-5 cells will be pre-incubated with purified PLAD-DOM7h-8 (or the DOM7h-8-PLAD) fusion protein such that the PLAD domain may form an inhibitory complex with TNFR1 on the cell surface. The cells will then be treated with human TNF-alpha, and incubated at 37° C. The amount of IL-8 that the MRC-5 cells secrete in response to TNF stimulation will then be measured using a IL-8 ELISA. PLAD activity of the fusion protein will be indicated by an inhibition of IL-8 secretion in a dose responsive fashion.

Anti-serum albumin activity: For analysis of PLAD-DOM7h-8 (or the DOM7h-8-PLAD) fusion protein affinity to serum albumin, a CM-5 BIAcore chip will be coupled to about 500 resonance units of albumin at pH5.5 and binding curves will be generated by flowing the purified fusion proteins diluted in BIAcore HBS-EP buffer in the range 5 nM to 5 μM across the BIAcore chip. Affinity (K_{D}) will be calculated by fitting on-rate and off-rate curves for traces generated in the range of the K_{D} for each fusion protein, and will be compared to the affinity of DOM7h-8 (IQ: 70 nM for human serum albumin) in the absence of fusion partner (as a separate molecular entity).

Pharmacokinetic study: Groups of 4 rats will be given an intravenous bolus of 1.5 mg/kg of fusion protein or control immunoglobulin variable domain that binds serum albumin (both will be radio-labelled with [³H]-NSP) and serum samples will be obtained from a tail vein over a 7 day period for radioactive count analysis. Serum concentration vs time curves will be fitted for a 1 or 2 compartment(s) model using WinNonlin software. Terminal half-life in the order of 15 hours will be expected for the fusion protein, provided that the PLAD moiety does not influence the terminal half-life of the immunoglobulin variable domain that binds serum albumin.

TABLE 9

| | | | | | Plasmid |
|---|---|---|---|---|---|
| | Primers | | Primers | Primers for | to be |
| Tem- | for PCR | Tem- | for PCR | SOE-PCR of | ligated |
| plate 1 | of 1 | plate 2 | of 2 | 1 with 2 | into |
| DOM7h-8 | DOM008, | PLAD | 1398, 1400 | DOM008, | pUC119 |
| | 1399 | | | 1400 | |
| DOM7h-8 | VK | PLAD | 1398, 1400 | VK | pPICZα |
| | EAEA, | | | EAEA, | |
| | 1399 | | | 1400 | |
| DOM7h-8 | 1393, | PLAD | 1398, 1401 | 1393, 1401 | pDOM32 |
| | 1399 | | | | |

TABLE 10

| Primer | |
|---|---|
| Name | Sequence |
| DOM008 | AGCGGATAACAATTTCACACAGGA |
| | (SEQ ID NO:101) |
| VK | TATCTCGAGAAAAGAGAGGCTGAAGCAGACATCCAGATGACC |
| EAEA | CAGTCTC |
| (or VK) | (SEQ ID NO:102) |
| | (TATCTCGAGAAAAGAGACATCCAGATGACCCAGTCTC |
| | (SEQ ID NO:103) |
| 1393 | CCCGGATCCACCGGCGACATCCAGATGACCCAGTCTC |
| | (SEQ ID NO:104) |
| 1399 | GAGGGACCAGAGATGGAGCAGCGACGGTCCGTTTGATTTCCA |
| | CCTTGGTCCC |
| | (SEQ ID NO:105) |
| 1398 | CAAACGGACCGTCGCTGCTCCATCTCTGGTCCCTCACCTAGG |
| | GGACAG |
| | (SEQ ID NO:106) |
| 1400 | GCGACAGGGAGCGGCCGCTCATTACCTGCAGTCCGTATCCTG |
| | CCCC |
| | (SEQ ID NO:107) |
| 1401 | GACAGAAGCTTATCACCTGCAGTCCGTATCCTGCCCC |
| | (SEQ ID NO:108) |

While this invention, has been particularly shown and described with references to preferred embodiments thereof, it will be understood by those skilled in the art that various changes in form and details may be made therein without departing from the scope of the invention encompassed by the appended claims.

1. A drug fusion comprising moieties X′ and Y′, wherein
X′ is a PLAD domain or functional variant of a PLAD domain; and
Y′ is a polypeptide binding moiety having a binding site that has binding specificity for a polypeptide that enhances serum half-life in vivo.

2. The drug fusion of claim 1 wherein said polypeptide binding moiety has binding specificity for serum albumin.

3. The drug fusion of claim 1 wherein said polypeptide binding moiety is an antigen-binding fragment of an antibody that has binding specificity for serum albumin.

4. The drug fusion of claim 1 wherein said PLAD domain or functional variant of a PLAD domain comprises a region of at least about 10 contiguous amino acids that are the same as the amino acids in the amino acid sequence of a PLAD domain selected from the PLAD domains of TNFR1, TNFR2, FAS, LT βR, CD40, CD30, CD27, HVEM, OX40, and DR4.

5. The drug fusion of claim 4 wherein the amino acid sequence of the PLAD domain or functional variant of a PLAD domain has at least about 90% amino acid sequence identity with the amino acid sequence of a PLAD domain selected from the PLAD domains of TNFR1, TNFR2, FAS, LT βR, CD40, CD30, CD27, HVEM, OX40, and DR4.

6. The drug fusion of claim 5 wherein the amino acid sequence of said PLAD domain or functional variant of a PLAD domain has at least about 90% amino acid sequence identity with an amino acid sequence selected from the group consisting of SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97.

7. A drug fusion comprising moieties X′ and Y′, wherein
X′ is a PLAD domain or functional variant of a PLAD domain; and
Y′ is an immunoglobulin heavy chain variable domain that has binding specificity for serum albumin, or an immunoglobulin light chain variable domain that has binding specificity for serum albumin.

8. The drug fusion of claim 7, wherein X′ is located amino terminally to Y′.

9. The drug fusion of claim 7, wherein Y′ is located amino terminally to X′.

10. The drug fusion of claim 7 wherein the heavy chain variable domain and the light chain variable domain have binding specificity for human serum albumin.

11. The drug fusion of claim 10 wherein Y′ comprises an amino acid sequence selected from the group consisting of SEQ ID NO:10, SEQ ID NO:11, SEQ ID NO:12, SEQ ID NO: 13, SEQ ID NO: 14, SEQ ID NO: 15, SEQ ID NO:24, SEQ ID NO:25 and SEQ ID NO:26.

12. The drug fusion of claim 10 wherein Y′ comprises an amino acid sequence selected from the group consisting of SEQ ID NO: 16, SEQ ID NO: 17, SEQ ID NO: 18, SEQ ID NO:19, SEQ ID NO:20, SEQ ID NO:21, SEQ ID NO:22 and SEQ ID NO:23.

13. The drug fusion of claim 7 wherein said PLAD domain or functional variant of a PLAD domain comprises a region of at least about 10 contiguous amino acids that are the same as the amino acids in the amino acid sequence of a PLAD domain selected from the PLAD domains of TNFR1, TNFR2, FAS, LT PR, CD40, CD30, CD27, HVEM, OX40, and DR4.

14. The drug fusion of claim 13 wherein the amino acid sequence of the PLAD domain or functional variant of a PLAD domain has at least about 90% amino acid sequence identity with the amino acid sequence of a PLAD domain selected from the PLAD domains of TNFR1, TNFR2, FAS, LT βR, CD40, CD30, CD27, HVEM, OX40, and DR4.

15. The drug fusion of claim 14 wherein the amino acid sequence of said PLAD domain or functional variant of a PLAD domain has at least about 90% amino acid sequence identity with an amino acid sequence selected from the group consisting of SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97.

16. A drug conjugate comprising an immunoglobulin heavy chain variable domain that has binding specificity for serum albumin, or an immunoglobulin light chain variable domain that has binding specificity for serum albumin, and a PLAD domain or functional variant of a PLAD domain that is covalently bonded to said immunoglobulin heavy chain variable domain or immunoglobulin light chain variable domain.

17. The drug conjugate of claim 16, wherein the PLAD domain or functional variant of a PLAD domain is covalently bonded to said immunoglobulin heavy chain variable domain or immunoglobulin light chain variable domain through a linker moiety.

18. The drug conjugate of claim 16 wherein the immunoglobulin heavy chain variable domain that has binding specificity for serum albumin, or the immunoglobulin light chain variable domain that has binding specificity for serum albumin comprises an amino acid sequence selected from the group consisting of SEQ ID NO: 10, SEQ ID NO:11, SEQ ID NO: 12, SEQ ID NO: 13, SEQ ID NO: 14, SEQ ID NO: 15, SEQ ID NO:24, SEQ ID NO:25, SEQ ID NO:26, SEQ ID NO:16, SEQ ID NO:17, SEQ ID NO:18, SEQ ID NO:19, SEQ ID NO:20, SEQ ID NO:21, SEQ ID NO:22 and SEQ ID NO:23.

19. The drug conjugate of claim 16 wherein said PLAD domain or functional variant of a PLAD domain comprises a region of at least about 10 contiguous amino acids that are the same as the amino acids in the amino acid sequence of a PLAD domain selected from the PLAD domains of TNFR1, TNFR2, FAS, LT βR, CD40, CD30, CD27, HVEM, OX40, and DR4.

20. The drug conjugate of claim 19 wherein the amino acid sequence of the PLAD domain or functional variant of a PLAD domain has at least about 90% amino acid sequence identity with the amino acid sequence of a PLAD domain selected from the PLAD domains of TNFR1, TNFR2, FAS, LT βR, CD40, CD30, CD27, HVEM, OX40, and DR4.

21. The drug conjugate of claim 20 wherein the amino acid sequence of said PLAD domain or functional variant of a PLAD domain has at least about 90% amino acid sequence identity with an amino acid sequence selected from the group consisting of SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, and SEQ ID NO:97.

22. An isolated or recombinant nucleic acid that encodes a drug fusion according to claim 1.

23. A nucleic acid construct comprising the recombinant nucleic acid of claim 22.

24. A host cell comprising the recombinant nucleic acid of claim 22.

25. A method for producing a drug fusion comprising maintaining the host cell of claim 24 under conditions suitable for expression of said recombinant nucleic acid, whereby a drug fusion is produced.

26. A pharmaceutical composition comprising a drug fusion of claim 1 and a physiologically acceptable carrier.

27. A method for treating an individual having an inflammatory disease, comprising administering to said individual a therapeutically effective amount of a drug fusion of claim 1.

28. The method of claim 27, wherein the inflammatory disease is arthritis.

29-31. (canceled)

32. A method of treating lung inflammation or a respiratory disease in a subject, comprising locally administering to said subject an effective amount of a drug conjugate or drug fusion of claim 1.

33. A drug composition comprising a PLAD domain or functional variant of a PLAD domain that is bonded to a polypeptide binding moiety having a binding site that has binding specificity for a polypeptide that enhances serum half-life in vivo, wherein said drug composition has a longer in vivo serum half-life relative to said PLAD domain or functional variant of a PLAD domain, and has at least about 90% of the activity of the said PLAD domain or functional variant of a PLAD domain.

34. A drug fusion comprising a first moiety and a second moiety, wherein the first moiety is a PLAD domain or functional variant of a PLAD domain and the second moiety is a polypeptide that extends serum half-life in vivo.

35. A drug conjugate comprising a PLAD domain or functional variant of a PLAD domain that is conjugated to a polypeptide that extends serum half-life in vivo.

36. A pharmaceutical composition comprising a drug conjugate of claim 16 and a physiologically acceptable carrier.

37. A method for treating an individual having an inflammatory disease, comprising administering to said individual a therapeutically effective amount of a drug conjugate of claim 16.

38. The method of claim 37, wherein the inflammatory disease is arthritis.


SEQUENCE LISTING (108 sequences)

SEQ ID NO: 1 - protein, 108 aa, Homo sapiens
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 2 - protein, 108 aa, Homo sapiens
DIQMTQSPSSLSASVGDRVTITCRASQSIFRHLKWYQQKPGKAPKLLIYAASRLQSGVPSRFSGSGSGTDFTLTISSLQPEDFATYYCQQVALYPKTFGQGTKVEIKR

SEQ ID NO: 3 - protein, 108 aa, Homo sapiens
DIQMTQSPSSLSASVGDRVTITCRASQSIYYHLKWYQQKPGKAPKLLIYKASTLQSGVPSRFSGSGSGTDFTLTISSLQPEDFATYYCQQVRKVPRTFGQGTKVEIKR

SEQ ID NO: 4 - protein, 108 aa, Homo sapiens
DIQTTQSPSSLSASVGDRVTITCRASQYIGRYLRWYQQKPGKAPKLLIYDSSVLQSGVPSRFSGSGSGTDFTLTISSLQPEDFATYYCQQRYRMPYTFGQGTRVEIKR

SEQ ID NO: 5 - protein, 108 aa, Homo sapiens
DIQMTQSPSSLSASVGDRVTITCRASQYIGRYLRWYQQKPGKAPKLLIYDSSVLQSGVPSRFSGSGSGTDFTLTISSLQPEDFATYYCQQRYMQPFTFGQGTKVEIKR

SEQ ID NO: 6 - protein, 108 aa, Homo sapiens
DIQMTQSPSSLSASVGDRVTITCRASQWIGRYLRWYQQKPGKAPKLLIYNGSQLQSGVPSRFSGSGSGTDFTLTISSLQPEDFATYYCQQRYLQPYTFGQGTKVEIKR

SEQ ID NO: 7 - protein, 108 aa, Homo sapiens
DIQMTQSPSSLSASVGDRVTITCRASQYISRQLRWYQQKPGKAPRLLIYGASVLQSGIPSRFSGSGSGTDFTLTISSLQPEDFATYYCQQRYITPYTFGQGTKVEVKR

SEQ ID NO: 8 - protein, 108 aa, Homo sapiens
DIQMTQSPSSLSASVGDRVTITCRASQYIGRYLRWYQQKPGKAPKLLIYDSSVLQSGVPSRFSGSGSGTDFTLTISSLQPEDFATYYCQQRYSSPYTFGQGTKVEIKR

SEQ ID NO: 9 - protein, 108 aa, Homo sapiens
DIQMTQSPSSLSASVGDRVTITCRASQWIHRQLKWYQQKPGKAPKLLIYYASILQSGVPSRFSGSGSGTDFTLTISSLQPEDFATYYCQQTFSKPSTFGQGTKVEIKR

SEQ ID NO: 10 - protein, 108 aa, Homo sapiens
DIQMTQSPSSLSASVGDRVTITCRASQKIATYLNWYQQKPGKAPKLLIYRSSSLQSAVPSRFSGSGSGTVFTLTISSLQPEDFATYYCQQTYAVPPTFGQGTKVEIKR

SEQ ID NO: 11 - protein, 108 aa, Homo sapiens
DIQMTQSPSSLSASVGDRVTITCRASQWIDTGLAWYQQKPGKAPRLLIYNVSRLQSGVPSRFSGSGSGTDFTLTISSLQPEDFATYYCQQYWGSPTTFGQGTKVEIKR

SEQ ID NO: 12 - protein, 108 aa, Homo sapiens
DIQMTQSPSSLSASVGDRVTITCRASQEIYSWLAWYQQRPGKAPKLLIYNASHLQSGVPSRFSGSGSGTDFTLTISSLQPEDFATYYCQQVIGDPVTFGQGTKVEIKR

SEQ ID NO: 13 - protein, 108 aa, Homo sapiens
DIQMTQSPSSLSASVGDRVTITCRASQSISSYLNWYQQKPGKAPTLLIYRLSVLQSGVPSRFSGSGSGTDFTLTISSLQPEDFATYYCQQTYNVPPTFGQGTKVEIKR

SEQ ID NO: 14 - protein, 108 aa, Homo sapiens
DIQMTQSPSSLSASVGDRVTITCRASQSISSYLNWYQQKPGKAPKLLIYRNSFLQSGVPSRFSGSGSGTDFTLTISSLQPEDFATYYCQQTYTVPPTFGQGTKVEIKQ

SEQ ID NO: 15 - protein, 108 aa, Homo sapiens
DIQMTQSPSSLSASVGDRVTITCRASQSISSYLNWYQQKPGKAPKLLIYRNSQLQSGVPSRFSGSGSGTDFTLTISSLQPEDFATYYCQQTFAVPPTFGQGTKVEIKR

SEQ ID NO: 16 - protein, 123 aa, Homo sapiens
EVQLLESGGGLVQPGGSLRLSCAASGFTFSKYWMSWVRQAPGKGLEWVSSIDFMGPHTYYADSVKGRFTISRDNSKNTLYLQMNSLRAEDTAVYYCAKGRTSMLPMKGKFDYWGQGTLVTVSS

SEQ ID NO: 17 - protein, 118 aa, Homo sapiens
EVQLLESGGGLVQPGGSLRLSCAASGFTFYDYNMSWVRQAPGKGLEWVSTITHTGGVTYYADSVKGRFTISRDNSKNTLYLQMNSLRAEDTAVYYCAKQNPSYQFDYWGQGTLVTVSS

SEQ ID NO: 18 - protein, 118 aa, Homo sapiens
EVQLLESGGGLVQPGGSLRLSCAASGFTFHRYSMSWVRQAPGKGLEWVSTILPGGDVTYYADSVKGRFTISRDNSKNTLYLQMNSLRAEDTAVYYCAKQTPDYMFDYWGQGTLVTVSS

SEQ ID NO: 19 - protein, 117 aa, Homo sapiens
EVQLLESGGGLVQPGGSLRLSCAASGFTFWKYNMAWVRQAPGKGLEWVSTILGEGNNTYYADSVKGRFTISRDNSKNTLYLQMNSLRAEDTAVYYCAKTMDYKFDYWGQGTLVTVSS

SEQ ID NO: 20 - protein, 118 aa, Homo sapiens
EVQLLESGGGLVQPGGSLRLSCTASGFTFDEYNMSWVRQAPGKGLEWVSTILPHGDRTYYADSVKGRFTISRDNSKNTLYLQMNSLRAEDTAVYYCAKQDPLYRFDYWGQGTLVTVSS

SEQ ID NO: 21 - protein, 120 aa, Homo sapiens
EVQLLESGGGLVQPGGSLRLSCAASGFTFDLYDMSWVRQAPGKGLEWVSSIVNSGVRTYYADSVKGRFTISRDNSKNTLYLQMNSLRAEDTAVYYCAKLNQSYHWDFDYWGQGTLVTVSS

SEQ ID NO: 22 - protein, 118 aa, Homo sapiens
EVQLLESGGGLVQPGGSLRLSCAASGFTFSDYRMSWVRQAPGKGLEWVSTIISNGKFTYYADSVKGRFTISRDNSKNTLYLQMNSLRAEDTAVYYCAKQDWMYMFDYWGQGTLVTVSS

SEQ ID NO: 23 - protein, 35 aa, Artificial Sequence
  consensus sequence
EVQLLESGGGLVQPGGSLRLSCAASGFTFXXYNMS

SEQ ID NO: 24 - protein, 108 aa, Homo sapiens
DIQMTQSPSSLSASVGDRVTITCRASQSISSYLNWYQQKPGKAPKLLIYRNSPLQSGVPSRFSGSGSGTDFTLTISSLQPEDFATYYCQQTYRVPPTFGQGTKVEIKR

SEQ ID NO: 25 - protein, 108 aa, Homo sapiens
DIQMTQSPSSLSASVGDRVTITCRASQHIHRELRWYQQKPGKAPKLLIYQASRLQSGVPSRFSGSGSGTDFTLTISSLQPEDFATYYCQQKYLPPYTFGQGTKVEIKR

SEQ ID NO: 26 - protein, 108 aa, Homo sapiens
DIQMTQSPSSLSASVGDRVTITCRASQHIHRELRWYQQKPGKAPKLLIYQASRLQSGVPSRFSGSGSGTDFTLTISSLQPEDFATYYCQQRYRVPYTFGQGTKVEIKR

SEQ ID NO: 27 - DNA, 877 bp, Artificial Sequence
  Encodes IL-1ra/anti-mouse serum albumin dAb fusion protein
aggccttctgggagaaaatccagcaagatgcaagccttcagaatctgggatgttaaccagaagaccttctatctgaggaacaaccaactagttgccggatacttgcaaggaccaaatgtcaatttagaagaaaagatagatgtggtaccattgagcctcatgctctgttcttgggaatccatggagggaagatctgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagagctcaggtggaggcggttcaggcggaggtggcagcggcggtggcgggtcaggtggtggcggaagcggcggtggcgggtcgacggacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacaggggctcggtggcctcagacgttcggccaagggaccaaggtggaaatcaaacgggcggccgcataataa

SEQ ID NO: 28 - protein, 290 aa, Artificial Sequence
  IL-1ra /anti-mouse serum albumin dAb fusion protein
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDESSGGGGSGGGGSGGGGSGGGGSGGGGSTDIQMTQSPSSLSASVGDRVTITCRASQSIIKHLKWYQQKPGKAPKLLIYGASRLQSGVPSRFSGSGGTDFTLTISSLQPEDFATYYCQQGARWPQTFGQGTKVEIKRAAA

SEQ ID NO: 29 - DNA, 878 bp, Artificial Sequence
  encodes anti-mouse serum albumin dAb/IL-1ra fusion protein
tcgacggacatccagatgacccagtctccatcctcctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgggcggccgcaagcggtggaggcggttcaggcggaggtggcagcggcggtggcgggtcaggtggtggcggaagcggcggtggcggctcgaggccctctgggagaaaatccagcaagatgcaagccttcagaatctgggatgttaaccagaagaccttctatctgaggaacaaccaactagttgccggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtaataa

SEQ ID NO: 30 - protein, 291 aa, Artificial Sequence
  anti-mouse serum albumin dAb/IL-1ra fusion protein
STDIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKRAAASGGGGSGGGGSGGGGSGGGGSGGGGSRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 31 - DNA, 878 bp, Artificial Sequence
  encodes dummy dAb/IL-1ra fusion protein
tcgacggacatccagatgacccagtctccatcctccctgtctgcatctgtaggagaccgtgtcaccatcacttgccgggcaagtcagagcattagcagctatttaaattggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagagttacagtacccctaatacgttcggccaagggaccaaggtggaaatcaaacgggcggccgcaagcggtggaggcggttcaggcggaggtggcagcggcggtggcgggtcaggtggtggcggaagcgcggtggcggctcgaggccctctgggagaaaatccagcaagatgcaagccttcagaatctgggatgttaaccagaagaccttctatctgaggaacaaccaactagttgccggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtaataa

SEQ ID NO: 32 - protein, 291 aa, Artificial Sequence
  dummy dAb/IL-1ra fusion protein
STDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKRAAASGGGGSGGGGSGGGGSGGGGSGGGGSRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 33 - DNA, 1759 bp, Homo sapiens
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaa

SEQ ID NO: 34 - protein, 178 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESSAACPGWFLCTAMEADQPVSLTNMPDEGVMVTKFYFQEDE

SEQ ID NO: 35 - DNA, 73 bp, Artificial Sequence
  Multiple cloning site
gcgcatatgttagtgcgtcgacgtcaaaaggccatagcgggcggccgctgcaggtctcgagtgcgatggatcc

SEQ ID NO: 36 - DNA, 92 bp, Artificial Sequence
  Multiple cloning site
gcgcatatgttaagcgaggccttctggagagagctcaggagtgtcgacggacatccagatgacccaggcggccgctaataaggatccaatgc

SEQ ID NO: 37 - protein, 108 aa, Homo sapiens
DIQMTQSPSSLSASVGDRVTITCRASQSIGRRLKWYQQKPGAAPRLLIYRTSWLQSGVPSRFSGSGSGTDFTLTISSLQPEDFATYYCQQTSQWPHTFGQGTKVEIKR

SEQ ID NO: 38 - protein, 108 aa, Homo sapiens
DIQMTQSPSSLSASVGDRVTITCRASQKIYKNLRWYQQKPGKAPKLLIYNSSILQSGVPSRFSGSGSGTDFTLTISSLQPEDFATYYCQQRYLSPYTFGQGTKVEIKR

SEQ ID NO: 39 - protein, 108 aa, Homo sapiens
DIQMTQSPSSLSASVGDRVTITCRASQKIYNNLRWYQQKPGKAPKLLIYNTSILQSGVPSRFSGSGSGTDFTLTISSLQPEDFATYYCQQRWRAPYTFGQGTKVEIKR

SEQ ID NO: 40 - protein, 108 aa, Homo sapiens
DIQMTQSPSSLSASVGDRVTITCRASQWIYKSLGWYQQKPGKAPKLLIYQSSLLQSGVPSRFSGSGSGTDFTLTISSLQPEDFATYYCQQYHQMPRTFGQGTKVEIKR

SEQ ID NO: 41 - protein, 108 aa, Homo sapiens
DIQMTQSPSSLSASVGDRVTITCRASQWIYRHLRWYQQKPGKAPKLLIYDASRLQSGVPTRFSGSGSGTDFTLTISSLQPEDFATYYCQQTHNPPKTFGQGTKVEIKR

SEQ ID NO: 42 - protein, 116 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKGGKDFDYWGQGTLVTVSS

SEQ ID NO: 43 - protein, 117 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKGNLEPFDYWGQGTLVTVSS

SEQ ID NO: 44 - protein, 117 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKKLSNGFDYWGQGTLVTVSS

SEQ ID NO: 45 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKVVKDNTFDYWGQGTLVTVSS

SEQ ID NO: 46 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKNTGGKQFDYWGQGTLVTVSS

SEQ ID NO: 47 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKKTGPSSFDYWGQGTLVTVSS

SEQ ID NO: 48 - protein, 120 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKRTENRGVSFDYWGQGTLVTVSS

SEQ ID NO: 49 - protein, 122 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKSDVLKTGLDGFDYWGQGTLVTVSS

SEQ ID NO: 50 - protein, 120 aa, Homo sapiens
EVQLLESGGGLVQPGGSLRLSCAASGFTFMAYQMAWVRQAPGKGLEWVSTIHQTGFSTYYADSVKGRFTISRDNSKNTLYLQMNSLRAEDTAVYYCAKVRSMRPYKFDYWGQGTLVTVSS

SEQ ID NO: 51 - protein, 120 aa, Homo sapiens
EVQLLESGGGLVQPGGSLRLSCAASGFTFKDYDMTWVRQAPGKGLEWVSMISSSGLWTYYADSVKGRFTISRDNSKNTLYLQMNSLRAEDTAVYYCAKGFRLFPRTFDYWGQGTLVTVSS

SEQ ID NO: 52 - protein, 121 aa, Homo sapiens
EVQLLESGGGLVQPGGSLRLSCAASGFTFHDYVMGWARQAPGKGLEWVSLIKPNGSPTYYADSVKGRFTISRDNSKNTLYLQMNSLRAEDTAVYYCAKGRGRFNVLQFDYWGQGTLVTVSS

SEQ ID NO: 53 - protein, 118 aa, Homo sapiens
EVQLLESGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYMGDRFDYWGQGTLVTVSS

SEQ ID NO: 54 - protein, 116 aa, Homo sapiens
EVQLLESGGGLVQPGGSLRLSCAASGFTFMWDKMGWVRQAPGKGLEWVSFIGREGYGTYYADSVKGRFTISRDNSKNTLYLQMNSLRAEDTAVYYCAKSVASFDYWGQGTLVTVSS

SEQ ID NO: 55 - protein, 117 aa, Homo sapiens
EVQLLESGGGLVQPGGSLRLSCAASGFTFWAYPMSWVRQAPGKGLEWVSSISSWGTGTYYADSVKGRFTISRDNSKNTLYLQMNSLRAEDTAVYYCAKGGQGSFDYWGQGTLVTVSS

SEQ ID NO: 56 - protein, 292 aa, Saponaria officinalis
MKIYVVATIAWILLQFSAWTTTDAVTSITLDLVNPTAGQYSSFVDKIRNNVKDPNLKYGGTDIAVIGPPSKDKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPKSSNEANSTAYATTVL

SEQ ID NO: 57 - protein, 236 aa, Saponaria officinalis
DPNLKYGGTDIAVIGPPSRDKFLRLNFQSSRGTVSLGLKRENLYVVAYLAMDNANVNRAYYFGTEITSAELTTLLPEATVANQKALEYTEDYQSIEKNAKITEGDKTRKELGLGINLLSTLMDAVNKKARVVKNEARFLLIAIQMTAEAARFRYIQNLVTKNFPNKFNSEDKVIQFQVNWSKISKAIYGDAKNGVFNKDYDFGFGKVRQVKDLQMGLLMYLGTTPNNAADRYRAEL

SEQ ID NO: 58 - protein, 157 aa, Saponaira officinalis
MKIYVVATIAWILLQFSAWTTTDAVTSITLDLVNPTAGQYSSFVDKIRNNVKDPNLKYGGTDIAVIGPPSKGKFLRINFQSSRGTVSLGLKRDNLYVVAYLAMDNTNVNRAYYFRSEITSAELTALFPEATTANQKALEYTEDYQSIEKNAQITQED

SEQ ID NO: 59 - protein, 253 aa, Saponaria officinalis
VTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAELTALFPEATTANQKALEYTEDYQSIEKNAQITQGDKSRKELGLGIDLLLTSMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPK

SEQ ID NO: 60 - protein, 299 aa, Saponaria officinalis
MKIYVVATIAWILLQFSAWTTTDAVTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPKSSNEANSTVRHYGPLKPTLLIT

SEQ ID NO: 61 - protein, 253 aa, Saponaria officinalis
VTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPK

SEQ ID NO: 62 - protein, 253 aa, Saponaria officinalis
VTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAELTALFPEATTANQKALEYTEDYQSIEKNAQITQGDKSRKELGLGIDLLLTSMEAVNKKARVVKNEARFLLIAIQMTAEAARFRYIQNLVIKNFPNKFNSENKVIQFEVNWKKISTAIYGDAKNGVFNKDYDFGFGKVRQVKDLQMGLLMYLGKPK

SEQ ID NO: 63 - protein, 275 aa, Artificial Sequence
  consensus sequence
VTSITLDLVNPTAGQYSSFVDKIRNNVKDPNLKYGGTDIAVIGPPSKXKFLRINFQSSRGTVSLGLKRDNLYVVAYLAMDNTNVNRAYYFXSEITSAEXTALFPEATTANQKALEYTEDYQSIEKNAQITQGDXSRKELGLGIDLLXTXMEAVNKKARVVKXEARFLLIAIQMTAEXARFRYIQNLVXKNFPNKFXSXNKVIQFEVXWXKISTAIYGDAKNGVFNKDYDFGFGKVRQVKDLQMGLLMYLGKPKSSNEANSTVYHYGPLKPTLLIT

SEQ ID NO: 64 - protein, 16 aa, Unknown
  Hemagglutinin tag peptide
YPYDVPDYAKKKKKKC

SEQ ID NO: 65 - protein, 16 aa, Unknown
  hemagglutinin tag peptide
CKKKKKKYPYDVPDYA

SEQ ID NO: 66 - protein, 13 aa, Artificial Sequence
  His tag peptide
HHHHHHKKKKKKC

SEQ ID NO: 67 - protein, 13 aa, Artificial Sequence
  His tag peptide
CKKKKKKHHHHHH

SEQ ID NO: 68 - protein, 117 aa, Unknown
  Camelid anti-mouse serum albumin
QVQLQESGGGLVQPGGSLRLSCEASGFTFTSRFGMGTWVRQAPGKGVEWVSGISSLGDSTLYADSVKGRFTISRDNAKNTLYLQMNSLKPEDTAVYYCTIGGSLNPGGQGTQVTVSS

SEQ ID NO: 69 - protein, 115 aa, Unknown
  Camelid anti-mouse serum albumin
QVQLQESGGGLVQPGNSLRLSCAASGFTFRNFGMSWVRQAPGKEPEWVSSISGSGSNTIYADSVKDRFTISRDNAKSTLYLQMNSLKPEDTAVYYCTIGGSLSRSSQGTQVTVSS

SEQ ID NO: 70 - protein, 114 aa, Unknown
  Camelid anti-mouse serum albumin
QVQLQESGGGLVQPGGSLRLTCTASGFTFSSFGMSWVRQAPGKGLEWVSAISSDSGTKNYADSVKGRFTISRDNAKKMLFLQMNSLRPEDTAVYYCVIGRGSPSSQGTQVTVSS

SEQ ID NO: 71 - protein, 114 aa, Unknown
  Camelid anti-mouse serum albumin
QVQLQESGGGLVQPGGSLRLTCTASCFTFRSFGMSWVRQAPGKGLEWVSAISADGSDKRYADSVKGRFTISRDNGKKMLTLDMNSLKPEDTAVYYCVIGRGSPASQGTQVTVSS

SEQ ID NO: 72 - protein, 128 aa, Unknown
  Camelid anti-mouse serum albumin
AVQLVESGGGLVQAGDSLRLSCVVSGTTFSSAAMGWFRQAPGKEREFVGAIKWSGTSTYYTDSVKGRFTISRDNVKNTVYLQMNNLKPEDTGVYTCAADRDRYRDRMGPMTTTDFRFWGQGTQVTVSS

SEQ ID NO: 73 - protein, 123 aa, Unknown
  Camelid anti-mouse serum albumin
QVKLEESGGLVQTGGSLRLSCAASGRTFSSFAMGWFRQAPGREREFVASIGSSGITTNYADSVKGRFTISRDNAKNTVYLQMNSLKPEDTGLCYCAVNRYGIPYRSGTQYQNWGQGTGVTVSS

SEQ ID NO: 74 - protein, 120 aa, Unknown
  Camelid anti-mouse serum albumin
EVQLEESGGGLVQPGGSLRLSCAASGLTFNDYAMGWYRQAPGKERDMVATISIGGRTYYADSVKGRFTISRDNAKNTVYLQMNSLKPEDTAIYYCVAHRQTVVRGPYLLWGQGTQVTVSS

SEQ ID NO: 75 - protein, 123 aa, Unknown
  Camelid anti-mouse serum albumin
QVQLVESGGKLVQAGGSLRLSCAASGRTFSNYAMGWFRQAPGKEREFVAGSGRSNSYNYYSDSVKGRFTISRDNAKNTVYLQMNSLKPEDTAVYYCAASTNLWPRDRNLYAYWGQGTQVTVSS

SEQ ID NO: 76 - protein, 125 aa, Unknown
  Camelid anti-mouse serum albumin
EVQLVESGGGLVQAGDSLRLSCAASGRSLGIYRMGWFRQVPGKEREFVAAISWSGGTTRYLDSVKGRFTISRDSTKNAVYLQMNSLKPEDTAVYYCAVDSSGRLYWTLSTSYDYWGQGTQVTVSS

SEQ ID NO: 77 - protein, 125 aa, Unknown
  Camelid anti-mouse serum albumin
QVQLVEFGGGLVQAGDSLRLSCAASGRSLGIYKMAWFRQVPGKEREFVAAISWSGGTTRYIDSVKGRFTLSRDNTKNMVYLQMNSLKPDDTAVYYCAVDSSGRLYWTLSTSYDYWGQGTQVTVSS

SEQ ID NO: 78 - protein, 124 aa, Unknown
  Camelid anti-mouse serum albumin
EVQLVESGGGLVQAGGSLSLSCAASGRTFSPYTMGWFRQAPGKEREFLAGVTWSGSSTFYGDSVKGRFTASRDSAKNTVTLEMNSLNPEDTAVYYCAAAYGGGLYRDPRSYDYWGRGTQVTVSS

SEQ ID NO: 79 - protein, 131 aa, Unknown
  Camelid anti-mouse serum albumin
AVQLVESGGGLVQAGGSLRLSCAASGFTLDAWPIAWFRQAPGKEREGVSCIRDGTTYYADSVKGRFTISSDNANNTVYLQTNSLKPEDTAVYYCAAPSGPATGSSHTFGIYWNLRDDYDNWGQGTQVTVSS

SEQ ID NO: 80 - protein, 126 aa, Unknown
  Camelid anti-mouse serum albumin
EVQLVESGGGLVQAGGSLRLSCAASGFTFDHYTIGWFRQVPGKEREGVSCISSSDGSTYYADSVKGRFTISSDNAKNTVYLQMNTLEPDDTAVYYCAAGGLLLRVEELQASDYDYWGQGIQVTVSS

SEQ ID NO: 81 - protein, 128 aa, Unknown
  Camelid anti-mouse serum albumin
AVQLVDSGGGLVQPGGSLRLSCTASGFTLDYYAIGWFRQAPGKEREGVACISNSDGSTYYGDSVKGRFTISRDNAKTTVYLQMNSLKPEDTAVYYCATADRHYSASHHPFADFAFNSWGQGTQVTVSS

SEQ ID NO: 82 - protein, 120 aa, Unknown
  Camelid anti-mouse serum albumin
EVQLVESGGGLVQAGGSLRLSCAAYGLTFWRAAMAWFRRAPGKERELVVARNWGDGSTRYADSVKGRFTISRDNAKNTVYLQMNSLKPEDTAVYYCAAVRTYGSATYDIWGQGTQVTVSS

SEQ ID NO: 83 - protein, 122 aa, Unknown
  Camelid anti-mouse serum albumin
EVQLVESGGGLVQDGGSLRLSCIFSGRTFANYAMGWFRQAPGKEREFVAAINRNGGTTNYADALKGRFTISRDNTKNTAFLQMNSLKPDDTAVYYCAAREWPFSTIPSGWRYGQGTQVTVSS

SEQ ID NO: 84 - protein, 125 aa, Unknown
  Camelid anti-mouse serum albumin
DVQLVESGGGWVQPGGSLRLSCAASGPTASSHAIGWFRQAPGKEREFVVGINRGGVTRDYADSVKGRFAVSRDNVKNTVYLQMNRLKPEDSAIYICAARPEYSFTAMSKGDMDYWGKGTLVTVSS

SEQ ID NO: 85 - protein, 182 aa, Homo sapiens
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 86 - protein, 183 aa, Mus musculus
LVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDSGTA

SEQ ID NO: 87 - protein, 38 aa, Artificial Sequence
  PLAD domain TNFR1
CPQGKYIHPQNNSICCTKCHKGTYLYNDCPGPGQDTDC

SEQ ID NO: 88 - protein, 36 aa, Artificial Sequence
  PLAD domain TNFR2
CRLREYYDQTAQMCCSKCSPGQHAKVFCTKTSDTVC

SEQ ID NO: 89 - protein, 43 aa, Artificial Sequence
  Plad domain FAS
RLSSKSVNAQVTDINSKGLELRKTVTTVETQNLEGLHHDGQFC

SEQ ID NO: 90 - protein, 62 aa, Artificial Sequence
  PLAD domain FAS
RLSSKSVNAQVTDINSKGLELRKTVTTVETQNLEGLHHDGQFCHKPCPPGERKARDCTVNGD

SEQ ID NO: 91 - protein, 38 aa, Artificial Sequence
  PLAD domain LTbetaR
CRDQEKEYYEPQHRICCSRCPPGTYVSAKCSRIRDTVC

SEQ ID NO: 92 - protein, 34 aa, Artificial Sequence
  PLAD domain CD40
CREKQYLINSQCCSLCQPGQKLVSDCTEFTETEC

SEQ ID NO: 93 - protein, 41 aa, Artificial Sequence
  PLAD domain CD30
CHGNPSHYYDKAVRRCCYRCPMGLFPTQQCPQRPTDCRKQC

SEQ ID NO: 94 - protein, 36 aa, Artificial Sequence
  PLAD domain CD27
WWLCVLGTLVGLSATPAPKSCPERHYWAQGKLCCQM

SEQ ID NO: 95 - protein, 34 aa, Artificial Sequence
  PLAD domain HVEM
CKEDEYPVGSECCPKCSPGYRVKEACGELTGTVC

SEQ ID NO: 96 - protein, 34 aa, Artificial Sequence
  PLAD domain OX40
VGARRLGRGPCAALLLLGLGLSTVTGLHCVGDTY

SEQ ID NO: 97 - protein, 30 aa, Artificial Sequence
  PLAD domain DR4
ATIKLHDQSIGTQQWEHSPLGELCPPGSHR

SEQ ID NO: 98 - DNA, 159 bp, Homo Sapiens
ctggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagg

SEQ ID NO: 99 - DNA, 324 bp, Homo sapiens
gacatccagatgacccagtctccatcctccctgtctgcatctgtaggagaccgtgtcaccatcacttgccgggcaagtcagagcattagcagctatttaaattggtatcagcagaaaccagggaaagcccctaagctcctgatctatcggaattcccctttgcaaagtggggtcccatcacgtttcagtggcagtggatctgggacagatttcactctcaccatcagcagtctgcaacctgaagattttgctacgtactactgtcaacagacgtatagggtgcctcctacgttcggccaagggaccaaggtggaaatcaaacgg

SEQ ID NO: 100 - protein, 6 aa, Artificial Sequence
  flexible linker
TVAAPS

SEQ ID NO: 101 - DNA, 23 bp, Artificial Sequence
  primer
gcggataacaatttcacacagga

SEQ ID NO: 102 - DNA, 48 bp, Artificial Sequence
  primer
atctcgagaaaagagaggctgaagcagacatccagatgacccagtctc

SEQ ID NO: 103 - DNA, 36 bp, Artificial Sequence
  primer
atctcgagaaaagagacatccagatgacccagtctc

SEQ ID NO: 104 - DNA, 36 bp, Artificial Sequence
  primer
ccggatccaccggcgacatccagatgacccagtctc

SEQ ID NO: 105 - DNA, 52 bp, Artificial Sequence
  primer
gagggaccagagatggagcagcgacggtccgtttgatttccaccttggtccc

SEQ ID NO: 106 - DNA, 47 bp, Artificial Sequence
  primer
aaacggaccgtcgctgctccatctctggtccctcacctaggggacag

SEQ ID NO: 107 - DNA, 45 bp, Artificial Sequence
  primer
cgacagggagcggccgctcattacctgcagtccgtatcctgcccc

SEQ ID NO: 108 - DNA, 36 bp, Artificial Sequence
  primer
acagaagcttatcacctgcagtccgtatcctgcccc